SEQUENCE LISTING (226 sequences)

SEQ ID NO: 1 - protein, 10 aa, Homo sapiens
  SITE 5: note = phosphorylation site
NNAASVHRAA

SEQ ID NO: 2 - protein, 11 aa, Homo sapiens
  SITE 5: note = phosphorylation site
RNQGSSLLGRR

SEQ ID NO: 3 - protein, 14 aa, Homo sapiens
  SITE 5: note = phosphorylation site
RGKDTAEGTNEDRG

SEQ ID NO: 4 - protein, 11 aa, Homo sapiens
  SITE 5: note = phosphorylation site
WQQESSMELKE

SEQ ID NO: 5 - protein, 11 aa, Homo sapiens
  SITE 5: note = phosphorylation site
AERKTSEHSQG

SEQ ID NO: 6 - protein, 11 aa, Homo sapiens
  SITE 5: note = phosphorylation site
LELKTTRFHPN

SEQ ID NO: 7 - protein, 11 aa, Homo sapiens
  SITE 5: note = phosphorylation site
PNRQSSMYTVT

SEQ ID NO: 8 - protein, 12 aa, Homo sapiens
  SITE 5: note = phosphorylation site
SSMYTVTRMESM

SEQ ID NO: 9 - protein, 11 aa, Homo sapiens
  SITE 5: note = phosphorylation site
LKDESSRAWTE

SEQ ID NO: 10 - protein, 10 aa, Homo sapiens
  SITE 5: note = phosphorylation site
SRAWTESSIN

SEQ ID NO: 11 - protein, 9 aa, Homo sapiens
  SITE 5: note = phosphorylation site
AWTESSINT

SEQ ID NO: 12 - protein, 12 aa, Homo sapiens
  SITE 5: note = phosphorylation site
AHGDSQTPLSQL

SEQ ID NO: 13 - protein, 14 aa, Homo sapiens
  SITE 5: note = phosphorylation site
QTPLSQLASGRRDP

SEQ ID NO: 14 - protein, 13 aa, Homo sapiens
  SITE 5: note = phosphorylation site
SLQPQLTQRSGLQ

SEQ ID NO: 15 - protein, 12 aa, Homo sapiens
  SITE 5: note = phosphorylation site
TQDATQTSCSTQ

SEQ ID NO: 16 - protein, 11 aa, Homo sapiens
  SITE 5: note = phosphorylation site
DATQTSCSTQS

SEQ ID NO: 17 - protein, 11 aa, Homo sapiens
  SITE 5: note = phosphorylation site
QTSCSTQSDAS

SEQ ID NO: 18 - protein, 12 aa, Homo sapiens
  SITE 5: note = phosphorylation site
GQHCSDSQRTSS

SEQ ID NO: 19 - protein, 11 aa, Homo sapiens
  SITE 5: note = phosphorylation site
DSQRTSSAMSG

SEQ ID NO: 20 - protein, 15 aa, Homo sapiens
  SITE 5: note = phosphorylation site
GPPPSFEGKTNESSL

SEQ ID NO: 21 - protein, 13 aa, Homo sapiens
  SITE 5: note = phosphorylation site
KTNESSLSAKSEL

SEQ ID NO: 22 - protein, 11 aa, Homo sapiens
  SITE 5: note = phosphorylation site
NESSLSAKSEL

SEQ ID NO: 23 - protein, 13 aa, Homo sapiens
  SITE 5: note = phosphorylation site
KAHKSQDSLCVTL

SEQ ID NO: 24 - protein, 11 aa, Homo sapiens
  SITE 5: note = phosphorylation site
KSQDSLCVTLA

SEQ ID NO: 25 - protein, 11 aa, Homo sapiens
  SITE 5: note = phosphorylation site
VSAATTQTLEG

SEQ ID NO: 26 - protein, 11 aa, Homo sapiens
  SITE 5: note = phosphorylation site
LEGDSSEVGDD

SEQ ID NO: 27 - protein, 15 aa, Homo sapiens
  SITE 5: note = phosphorylation site
EFPFDSEGPTEPTST

SEQ ID NO: 28 - protein, 11 aa, Homo sapiens
  SITE 5: note = phosphorylation site
STGRSTTCMTS

SEQ ID NO: 29 - protein, 11 aa, Homo sapiens
  SITE 5: note = phosphorylation site
TSLKSTNPSVA

SEQ ID NO: 30 - protein, 10 aa, Homo sapiens
  SITE 5: note = phosphorylation site
RKADSVSSNH

SEQ ID NO: 31 - protein, 11 aa, Homo sapiens
  SITE 5: note = phosphorylation site
ADSVSSNHTLS

SEQ ID NO: 32 - protein, 11 aa, Homo sapiens
  SITE 5: note = phosphorylation site
PSLASSGVNGG

SEQ ID NO: 33 - protein, 12 aa, Homo sapiens
  SITE 5: note = phosphorylation site
LSILSKSSSQIR

SEQ ID NO: 34 - protein, 11 aa, Homo sapiens
  SITE 5: note = phosphorylation site
GRSLSSCSPQP

SEQ ID NO: 35 - protein, 11 aa, Homo sapiens
  SITE 5: note = phosphorylation site
DSAQTSNTDTT

SEQ ID NO: 36 - protein, 12 aa, Homo sapiens
  SITE 5: note = phosphorylation site
QTSNTDTTSASP

SEQ ID NO: 37 - protein, 10 aa, Homo sapiens
  SITE 5: note = phosphorylation site
PTNDTAANSA

SEQ ID NO: 38 - protein, 10 aa, Homo sapiens
  SITE 5: note = phosphorylation site
PPAETELQAM

SEQ ID NO: 39 - protein, 11 aa, Homo sapiens
  SITE 5: note = phosphorylation site
SASYSSGHSQG

SEQ ID NO: 40 - protein, 11 aa, Homo sapiens
  SITE 5: note = phosphorylation site
LEMKSTRYLQT

SEQ ID NO: 41 - protein, 11 aa, Homo sapiens
  SITE 5: note = phosphorylation site
SRLETTISTVV

SEQ ID NO: 42 - protein, 11 aa, Homo sapiens
  SITE 5: note = phosphorylation site
QVSLTSKKHSR

SEQ ID NO: 43 - protein, 11 aa, Homo sapiens
  SITE 5: note = phosphorylation site
HSRKSSSYSSS

SEQ ID NO: 44 - protein, 11 aa, Homo sapiens
  SITE 5: note = phosphorylation site
SSSYSSSSTTV

SEQ ID NO: 45 - protein, 10 aa, Homo sapiens
  SITE 5: note = phosphorylation site
RGLGSEAKEE

SEQ ID NO: 46 - protein, 16 aa, Homo sapiens
  SITE 5: note = phosphorylation site
REQRSQDSRMTFRERT

SEQ ID NO: 47 - protein, 12 aa, Homo sapiens
  SITE 5: note = phosphorylation site
VVRPSDSGLPSE

SEQ ID NO: 48 - protein, 12 aa, Homo sapiens
  SITE 5: note = phosphorylation site
SGLPSESGPSSG

SEQ ID NO: 49 - protein, 11 aa, Homo sapiens
  SITE 5: note = phosphorylation site
TTCMTSLKSTN

SEQ ID NO: 50 - protein, 10 aa, Homo sapiens
  SITE 5: note = phosphorylation site
KEAYSERPGG

SEQ ID NO: 51 - protein, 11 aa, Homo sapiens
  SITE 5: note = phosphorylation site
MDTCSSNLNNS

SEQ ID NO: 52 - protein, 13 aa, Homo sapiens
  SITE 5: note = phosphorylation site
VNGGTQLSILSKS

SEQ ID NO: 53 - protein, 11 aa, Homo sapiens
  SITE 5: note = phosphorylation site
GTLRTSISVER

SEQ ID NO: 54 - protein, 12 aa, Homo sapiens
  SITE 5: note = phosphorylation site
RSSVTETEADGK

SEQ ID NO: 55 - protein, 10 aa, Homo sapiens
  SITE 5: note = phosphorylation site
PLDNSMGDSD

SEQ ID NO: 56 - protein, 10 aa, Homo sapiens
  SITE 5: note = phosphorylation site
SMGDSDCLHK

SEQ ID NO: 57 - protein, 11 aa, Homo sapiens
  SITE 5: note = phosphorylation site
DSTQTSDTATN

SEQ ID NO: 58 - protein, 12 aa, Homo sapiens
  SITE 5: note = phosphorylation site
QTSDTATNSTLP

SEQ ID NO: 59 - protein, 11 aa, Homo sapiens
  SITE 5: note = phosphorylation site
TATNSTLPSAE

SEQ ID NO: 60 - protein, 11 aa, Homo sapiens
  SITE 5: note = phosphorylation site
DSTQTSDTATN

SEQ ID NO: 61 - protein, 12 aa, Homo sapiens
  SITE 5: note = phosphorylation site
QTSDTATNSTLP

SEQ ID NO: 62 - protein, 11 aa, Homo sapiens
  SITE 5: note = phosphorylation site
TATNSTLPSAE

SEQ ID NO: 63 - protein, 13 aa, Homo sapiens
  SITE 5: note = phosphorylation site
SSAMSGHSRSFIS

SEQ ID NO: 64 - protein, 10 aa, Homo sapiens
  SITE 5: note = phosphorylation site
GAILTDTSVK

SEQ ID NO: 65 - protein, 11 aa, Homo sapiens
  SITE 5: note = phosphorylation site
ILTDTSVKRND

SEQ ID NO: 66 - protein, 9 aa, Homo sapiens
  SITE 5: note = phosphorylation site
RNDLSIISG

SEQ ID NO: 67 - protein, 13 aa, Homo sapiens
  SITE 5: note = phosphorylation site
QMENSMGTLRTSI

SEQ ID NO: 68 - protein, 11 aa, Homo sapiens
  SITE 5: note = phosphorylation site
EAPRSSVTETE

SEQ ID NO: 69 - protein, 10 aa, Homo sapiens
  SITE 5: note = phosphorylation site
NLNNSIYKKL

SEQ ID NO: 70 - protein, 11 aa, Homo sapiens
  SITE 5: note = phosphorylation site
TINGSTHVLRD

SEQ ID NO: 71 - protein, 14 aa, Homo sapiens
  SITE 5: note = phosphorylation site
ILGQTVPCSTVLRD

SEQ ID NO: 72 - protein, 11 aa, Homo sapiens
  SITE 5: note = phosphorylation site
GNGYSSNGNTG

SEQ ID NO: 73 - protein, 11 aa, Homo sapiens
  SITE 5: note = phosphorylation site
GNGYSSNGNTG

SEQ ID NO: 74 - protein, 11 aa, Homo sapiens
  SITE 5: note = phosphorylation site
GNGYSSNGNTG

SEQ ID NO: 75 - protein, 14 aa, Homo sapiens
  SITE 5: note = phosphorylation site
SNGNTGEQSGYHVE

SEQ ID NO: 76 - protein, 10 aa, Homo sapiens
  SITE 5: note = phosphorylation site
SNGNTGEQSG

SEQ ID NO: 77 - protein, 10 aa, Homo sapiens
  SITE 5: note = phosphorylation site
TGEQSGYHVE

SEQ ID NO: 78 - protein, 13 aa, Homo sapiens
  SITE 5: note = phosphorylation site
SLSSCSESSILTA

SEQ ID NO: 79 - protein, 12 aa, Homo sapiens
  SITE 5: note = phosphorylation site
ASRVSETEYSAL

SEQ ID NO: 80 - protein, 11 aa, Homo sapiens
  SITE 5: note = phosphorylation site
PERASSVYTRS

SEQ ID NO: 81 - protein, 10 aa, Homo sapiens
  SITE 5: note = phosphorylation site
SSVYTRSTGE

SEQ ID NO: 82 - protein, 10 aa, Homo sapiens
  SITE 5: note = phosphorylation site
VYTRSTGEQE

SEQ ID NO: 83 - protein, 10 aa, Homo sapiens
  SITE 5: note = phosphorylation site
APTETHSLSW

SEQ ID NO: 84 - protein, 10 aa, Homo sapiens
  SITE 5: note = phosphorylation site
CSAPTETHSL

SEQ ID NO: 85 - protein, 11 aa, Homo sapiens
  SITE 5: note = phosphorylation site
VSRGSSLKILS

SEQ ID NO: 86 - protein, 11 aa, Homo sapiens
  SITE 5: note = phosphorylation site
RGGHSSVSTES

SEQ ID NO: 87 - protein, 11 aa, Homo sapiens
  SITE 5: note = phosphorylation site
TESESSSFHSS

SEQ ID NO: 88 - protein, 11 aa, Homo sapiens
  SITE 5: note = phosphorylation site
HQWKSSEDNSK

SEQ ID NO: 89 - protein, 12 aa, Homo sapiens
  SITE 5: note = phosphorylation site
SEDNSKTFSASH

SEQ ID NO: 90 - protein, 12 aa, Homo sapiens
  SITE 5: note = phosphorylation site
SKTFSASHNVEA

SEQ ID NO: 91 - protein, 11 aa, Homo sapiens
  SITE 5: note = phosphorylation site
EEGGTTGASLS

SEQ ID NO: 92 - protein, 12 aa, Homo sapiens
  SITE 5: note = phosphorylation site
TTGASLSRFSYS

SEQ ID NO: 93 - protein, 12 aa, Homo sapiens
  SITE 5: note = phosphorylation site
LSRFSYSHMSAS

SEQ ID NO: 94 - protein, 11 aa, Homo sapiens
  SITE 5: note = phosphorylation site
EDPPTPSIASL

SEQ ID NO: 95 - protein, 12 aa, Homo sapiens
  SITE 5: note = phosphorylation site
LSRLSYTTISTL

SEQ ID NO: 96 - protein, 13 aa, Homo sapiens
  SITE 5: note = phosphorylation site
RNVLTEESVVRES

SEQ ID NO: 97 - protein, 12 aa, Homo sapiens
  SITE 5: note = phosphorylation site
VVRESKSFTRST

SEQ ID NO: 98 - protein, 10 aa, Homo sapiens
  SITE 5: note = phosphorylation site
SKSFTRSTVD

SEQ ID NO: 99 - protein, 11 aa, Homo sapiens
  SITE 5: note = phosphorylation site
SFTRSTVDTMA

SEQ ID NO: 100 - protein, 10 aa, Homo sapiens
  SITE 5: note = phosphorylation site
STVDTMAQKT

SEQ ID NO: 101 - protein, 12 aa, Homo sapiens
  SITE 5: note = phosphorylation site
DSKKSTSHDLVS

SEQ ID NO: 102 - protein, 11 aa, Homo sapiens
  SITE 5: note = phosphorylation site
MSIPTSPTRVS

SEQ ID NO: 103 - protein, 10 aa, Homo sapiens
  SITE 5: note = phosphorylation site
PTSPTRVSFH

SEQ ID NO: 104 - protein, 13 aa, Homo sapiens
  SITE 5: note = phosphorylation site
PTRVSFHSIKQST

SEQ ID NO: 105 - protein, 10 aa, Homo sapiens
  SITE 5: note = phosphorylation site
LCPATNNAIE

SEQ ID NO: 106 - protein, 11 aa, Homo sapiens
  SITE 5: note = phosphorylation site
AAMFSSHHEPR

SEQ ID NO: 107 - protein, 11 aa, Homo sapiens
  SITE 5: note = phosphorylation site
HGLHSTPDSPA

SEQ ID NO: 108 - protein, 12 aa, Homo sapiens
  SITE 5: note = phosphorylation site
SRTPVETVNISN

SEQ ID NO: 109 - protein, 10 aa, Homo sapiens
  SITE 5: note = phosphorylation site
SRIDTPPSTN

SEQ ID NO: 110 - protein, 10 aa, Homo sapiens
  SITE 5: note = phosphorylation site
LALVSLPEDS

SEQ ID NO: 111 - protein, 11 aa, Homo sapiens
  SITE 5: note = phosphorylation site
LPEDSSCRWAA

SEQ ID NO: 112 - protein, 10 aa, Homo sapiens
  SITE 5: note = phosphorylation site
RWAATPQDSS

SEQ ID NO: 113 - protein, 10 aa, Homo sapiens
  SITE 5: note = phosphorylation site
ATSLSQDNRK

SEQ ID NO: 114 - protein, 13 aa, Homo sapiens
  SITE 5: note = phosphorylation site
RYLQTQGSVYKVS

SEQ ID NO: 115 - protein, 13 aa, Homo sapiens
  SITE 5: note = phosphorylation site
YRHPSGGSNGATC

SEQ ID NO: 116 - protein, 13 aa, Homo sapiens
  SITE 5: note = phosphorylation site
SNGATCSTQVSML

SEQ ID NO: 117 - protein, 13 aa, Homo sapiens
  SITE 5: note = phosphorylation site
STQVSMLTRVSPG

SEQ ID NO: 118 - protein, 11 aa, Homo sapiens
  SITE 5: note = phosphorylation site
RVCGSSFSRNG

SEQ ID NO: 119 - protein, 14 aa, Homo sapiens
  SITE 5: note = phosphorylation site
GSSFSRNGSEGALQ

SEQ ID NO: 120 - protein, 14 aa, Homo sapiens
  SITE 5: note = phosphorylation site
FHRGSRAQSFLQTE

SEQ ID NO: 121 - protein, 10 aa, Homo sapiens
  SITE 5: note = phosphorylation site
IRQNTRDHPS

SEQ ID NO: 122 - protein, 10 aa, Homo sapiens
  SITE 5: note = phosphorylation site
RDHPSTANTV

SEQ ID NO: 123 - protein, 10 aa, Homo sapiens
  SITE 5: note = phosphorylation site
DHPSTANTVD

SEQ ID NO: 124 - protein, 10 aa, Homo sapiens
  SITE 5: note = phosphorylation site
STANTVDRTN

SEQ ID NO: 125 - protein, 10 aa, Homo sapiens
  SITE 5: note = phosphorylation site
VTACTPSDGP

SEQ ID NO: 126 - protein, 10 aa, Homo sapiens
  SITE 5: note = phosphorylation site
ACTPSDGPGG

SEQ ID NO: 127 - protein, 11 aa, Homo sapiens
  SITE 5: note = phosphorylation site
ERQSTSRVRNT

SEQ ID NO: 128 - protein, 10 aa, Homo sapiens
  SITE 5: note = phosphorylation site
RVRNTVQDPA

SEQ ID NO: 129 - protein, 10 aa, Homo sapiens
  SITE 5: note = phosphorylation site
DVQVSDRVRS

SEQ ID NO: 130 - protein, 10 aa, Homo sapiens
  SITE 5: note = phosphorylation site
DRVRSIAKDV

SEQ ID NO: 131 - protein, 11 aa, Homo sapiens
  SITE 5: note = phosphorylation site
LACKTSETVPR

SEQ ID NO: 132 - protein, 12 aa, Homo sapiens
  SITE 5: note = phosphorylation site
DGERSDSKQDKS

SEQ ID NO: 133 - protein, 11 aa, Homo sapiens
  SITE 5: note = phosphorylation site
RLNETTETQRT

SEQ ID NO: 134 - protein, 13 aa, Homo sapiens
  SITE 5: note = phosphorylation site
ETTETQRTLLNGD

SEQ ID NO: 135 - protein, 14 aa, Homo sapiens
  SITE 5: note = phosphorylation site
RRVRSQEPTVGPPE

SEQ ID NO: 136 - protein, 10 aa, Homo sapiens
  SITE 5: note = phosphorylation site
PPEKTEEEDE

SEQ ID NO: 137 - protein, 10 aa, Homo sapiens
  SITE 5: note = phosphorylation site
DGEESREGGK

SEQ ID NO: 138 - protein, 10 aa, Homo sapiens
  SITE 5: note = phosphorylation site
DADATEPRPD

SEQ ID NO: 139 - protein, 10 aa, Homo sapiens
  SITE 5: note = phosphorylation site
AAHGSDGEQV

SEQ ID NO: 140 - protein, 10 aa, Homo sapiens
  SITE 5: note = phosphorylation site
NNAASVHRAA

SEQ ID NO: 141 - protein, 10 aa, Homo sapiens
  SITE 5: note = phosphorylation site
RRHATHGDRP

SEQ ID NO: 142 - protein, 10 aa, Homo sapiens
  SITE 5: note = phosphorylation site
PLDNSMGDSD

SEQ ID NO: 143 - protein, 10 aa, Homo sapiens
  SITE 5: note = phosphorylation site
RPRASGCLAR

SEQ ID NO: 144 - protein, 10 aa, Homo sapiens
  SITE 5: note = phosphorylation site
SMGDSDCLHK

SEQ ID NO: 145 - protein, 11 aa, Homo sapiens
  SITE 5: note = phosphorylation site
DATQTSCSTQS

SEQ ID NO: 146 - protein, 12 aa, Homo sapiens
  SITE 5: note = phosphorylation site
RKLKTESEMHTL

SEQ ID NO: 147 - protein, 11 aa, Homo sapiens
  SITE 5: note = phosphorylation site
LKDESSRAWTE

SEQ ID NO: 148 - protein, 11 aa, Homo sapiens
  SITE 5: note = phosphorylation site
DVSKTSLKQAS

SEQ ID NO: 149 - protein, 13 aa, Homo sapiens
  SITE 5: note = phosphorylation site
SLQPQLTQRSGLQ

SEQ ID NO: 150 - protein, 11 aa, Homo sapiens
  SITE 5: note = phosphorylation site
INADSSSSFPN

SEQ ID NO: 151 - protein, 12 aa, Homo sapiens
  SITE 5: note = phosphorylation site
TQDATQTSCSTQ

SEQ ID NO: 152 - protein, 11 aa, Homo sapiens
  SITE 5: note = phosphorylation site
NNYASSSTSLP

SEQ ID NO: 153 - protein, 9 aa, Homo sapiens
  SITE 5: note = phosphorylation site
AWTESSINT

SEQ ID NO: 154 - protein, 12 aa, Homo sapiens
  SITE 5: note = phosphorylation site
AHGDSQTPLSQL

SEQ ID NO: 155 - protein, 12 aa, Homo sapiens
  SITE 5: note = phosphorylation site
KTSGSRTPGRYS

SEQ ID NO: 156 - protein, 12 aa, Homo sapiens
  SITE 5: note = phosphorylation site
SCSGTVSEQLRN

SEQ ID NO: 157 - protein, 12 aa, Homo sapiens
  SITE 5: note = phosphorylation site
DFSSSESQRSRH

SEQ ID NO: 158 - protein, 13 aa, Homo sapiens
  SITE 5: note = phosphorylation site
GSKYSEFTSTTSG

SEQ ID NO: 159 - protein, 11 aa, Homo sapiens
  SITE 5: note = phosphorylation site
GSGKTSGSRTP

SEQ ID NO: 160 - protein, 11 aa, Homo sapiens
  SITE 5: note = phosphorylation site
QTSCSTQSDAS

SEQ ID NO: 161 - protein, 11 aa, Homo sapiens
  SITE 5: note = phosphorylation site
YASSSTSLPCQ

SEQ ID NO: 162 - protein, 11 aa, Homo sapiens
  SITE 5: note = phosphorylation site
TIAMSTMHTDV

SEQ ID NO: 163 - protein, 11 aa, Homo sapiens
  SITE 5: note = phosphorylation site
GSVLSSNFTYH

SEQ ID NO: 164 - protein, 10 aa, Homo sapiens
  SITE 5: note = phosphorylation site
SRAWTESSIN

SEQ ID NO: 165 - protein, 12 aa, Homo sapiens
  SITE 5: note = phosphorylation site
LWEVSCSGTVSE

SEQ ID NO: 166 - protein, 10 aa, Homo sapiens
  SITE 5: note = phosphorylation site
AQIMTDFEKD

SEQ ID NO: 167 - protein, 11 aa, Homo sapiens
  SITE 5: note = phosphorylation site
PCQCSSTLMWS

SEQ ID NO: 168 - protein, 14 aa, Homo sapiens
  SITE 5: note = phosphorylation site
QTPLSQLASGRRDP

SEQ ID NO: 169 - protein, 12 aa, Homo sapiens
  SITE 5: note = phosphorylation site
QLRNSETKNLCL

SEQ ID NO: 170 - protein, 11 aa, Homo sapiens
  SITE 5: note = phosphorylation site
AKADTSKPSTV

SEQ ID NO: 171 - protein, 11 aa, Homo sapiens
  SITE 5: note = phosphorylation site
GGAGSSRRRRT

SEQ ID NO: 172 - protein, 14 aa, Homo sapiens
  SITE 5: note = phosphorylation site
ESQRSRHGSVLSSN

SEQ ID NO: 173 - protein, 10 aa, Homo sapiens
  SITE 5: note = phosphorylation site
NNAASVHRAA

SEQ ID NO: 174 - protein, 11 aa, Homo sapiens
  SITE 5: note = phosphorylation site
ADSSSSFPNGH

SEQ ID NO: 175 - protein, 13 aa, Homo sapiens
  SITE 5: note = phosphorylation site
STMHTDVSKTSLK

SEQ ID NO: 176 - protein, 12 aa, Homo sapiens
  SITE 5: note = phosphorylation site
SGKSTESSIGSG

SEQ ID NO: 177 - protein, 11 aa, Homo sapiens
  SITE 5: note = phosphorylation site
MHTLSSSAKAD

SEQ ID NO: 178 - protein, 10 aa, Homo sapiens
  SITE 5: note = phosphorylation site
RRHATHGDRP

SEQ ID NO: 179 - protein, 11 aa, Homo sapiens
  SITE 5: note = phosphorylation site
FTSTTSGTGHN

SEQ ID NO: 180 - protein, 11 aa, Homo sapiens
  SITE 5: note = phosphorylation site
RRRRTSSTARS

SEQ ID NO: 181 - protein, 15 aa, Homo sapiens
  SITE 5: note = phosphorylation site
TTSGTGHNQTRALRA

SEQ ID NO: 182 - protein, 12 aa, Homo sapiens
  SITE 5: note = phosphorylation site
VGSSSGHTSTTL

SEQ ID NO: 183 - protein, 10 aa, Homo sapiens
  SITE 5: note = phosphorylation site
SMGDSDCLHK

SEQ ID NO: 184 - protein, 10 aa, Homo sapiens
  SITE 5: note = phosphorylation site
RPRASGCLAR

SEQ ID NO: 185 - protein, 10 aa, Homo sapiens
  SITE 5: note = phosphorylation site
PLDNSMGDSD

SEQ ID NO: 186 - protein, 11 aa, Homo sapiens
  SITE 5: note = phosphorylation site
SFVGSSSGHTS

SEQ ID NO: 187 - protein, 10 aa, Homo sapiens
  SITE 5: note = phosphorylation site
DSRPSFVGSS

SEQ ID NO: 188 - protein, 12 aa, Homo sapiens
  SITE 5: note = phosphorylation site
  SITE 7: note = phosphorylation site
ASLASPSLRTSH

SEQ ID NO: 189 - protein, 11 aa, Homo sapiens
  SITE 5..6: note = phosphorylation site
PSLRTSHSGPR

SEQ ID NO: 190 - protein, 10 aa, Homo sapiens
  SITE 5: note = phosphorylation site
EEPGSEVVIK

SEQ ID NO: 191 - protein, 10 aa, Homo sapiens
  SITE 5: note = phosphorylation site
RDPVTENCVQ

SEQ ID NO: 192 - protein, 11 aa, Homo sapiens
  SITE 5..6: note = phosphorylation site
EEKESSNDSTS

SEQ ID NO: 193 - protein, 12 aa, Homo sapiens
  SITE 5..7: note = phosphorylation site
SSNDSTSVSAVA

SEQ ID NO: 194 - protein, 12 aa, Homo sapiens
  SITE 5: note = phosphorylation site
  SITE 7: note = phosphorylation site
QDENTVSTSLGH

SEQ ID NO: 195 - protein, 11 aa, Homo sapiens
  SITE 5..6: note = phosphorylation site
NTVSTSLGHSK

SEQ ID NO: 196 - protein, 15 aa, Homo sapiens
  SITE 5: note = phosphorylation site
  SITE 10: note = phosphorylation site
SLGHSKDENSKQTCI

SEQ ID NO: 197 - protein, 12 aa, Homo sapiens
  SITE 5..7: note = phosphorylation site
DQDHSSSDSWNN

SEQ ID NO: 198 - protein, 12 aa, Homo sapiens
  SITE 5: note = phosphorylation site
  SITE 7: note = phosphorylation site
EDIGSETRAIYS

SEQ ID NO: 199 - protein, 11 aa, Homo sapiens
  SITE 5..6: note = phosphorylation site
TSNESSSGSAT

SEQ ID NO: 200 - protein, 12 aa, Homo sapiens
  SITE 5: note = phosphorylation site
  SITE 8: note = phosphorylation site
ERPATELSTTEA

SEQ ID NO: 201 - protein, 10 aa, Homo sapiens
  SITE 5: note = phosphorylation site
AAHGSDGEQV

SEQ ID NO: 202 - protein, 11 aa, Homo sapiens
  SITE 5..6: note = phosphorylation site
HSNLSTKMSTL

SEQ ID NO: 203 - protein, 11 aa, Homo sapiens
  SITE 5..6: note = phosphorylation site
STKMSTLSYRP

SEQ ID NO: 204 - protein, 11 aa, Homo sapiens
  SITE 5..6: note = phosphorylation site
SDNVSSSTKKP

SEQ ID NO: 205 - protein, 12 aa, Homo sapiens
  SITE 5: note = phosphorylation site
  SITE 7: note = phosphorylation site
SGSSSYSYGPMV

SEQ ID NO: 206 - protein, 11 aa, Homo sapiens
  SITE 5..6: note = phosphorylation site
MVSHTSVTNVG

SEQ ID NO: 207 - protein, 11 aa, Homo sapiens
  SITE 5..6: note = phosphorylation site
PPKNSTDIQVQ

SEQ ID NO: 208 - protein, 10 aa, Homo sapiens
  SITE 5: note = phosphorylation site
RTPPSLGPQD

SEQ ID NO: 209 - protein, 11 aa, Homo sapiens
  SITE 5..6: note = phosphorylation site
DESCTTASSSL

SEQ ID NO: 210 - protein, 11 aa, Homo sapiens
  SITE 4..6: note = phosphorylation site
TTASSSLAKDT

SEQ ID NO: 211 - protein, 11 aa, Homo sapiens
  SITE 5..6: note = phosphorylation site
PVADTSLTTLA

SEQ ID NO: 212 - protein, 11 aa, Homo sapiens
  SITE 5..6: note = phosphorylation site
DTSLTTLAVMG

SEQ ID NO: 213 - protein, 10 aa, Homo sapiens
  SITE 5: note = phosphorylation site
AVMGTVPGTG

SEQ ID NO: 214 - protein, 13 aa, Homo sapiens
  SITE 5: note = phosphorylation site
  SITE 8: note = phosphorylation site
DAIHSEVSVTFKA

SEQ ID NO: 215 - protein, 12 aa, Homo sapiens
  SITE 5: note = phosphorylation site
  SITE 7: note = phosphorylation site
HSEVSVTFKAKK

SEQ ID NO: 216 - protein, 15 aa, Homo sapiens
  SITE 5: note = phosphorylation site
  SITE 10: note = phosphorylation site
VRKNSGPNDSFTEAT

SEQ ID NO: 217 - protein, 13 aa, Homo sapiens
  SITE 5: note = phosphorylation site
  SITE 8: note = phosphorylation site
STMHTDVSKTSLK

SEQ ID NO: 218 - protein, 11 aa, Homo sapiens
  SITE 5..6: note = phosphorylation site
DVSKTSLKQAS

SEQ ID NO: 219 - protein, 10 aa, Homo sapiens
  SITE 5: note = phosphorylation site
VIKESDHFST

SEQ ID NO: 220 - protein, 11 aa, Homo sapiens
  SITE 5..6: note = phosphorylation site
SDHFSTELDDI

SEQ ID NO: 221 - protein, 10 aa, Homo sapiens
  SITE 5: note = phosphorylation site
LDDITVTDTY

SEQ ID NO: 222 - protein, 11 aa, Homo sapiens
  SITE 5..6: note = phosphorylation site
TKLPSSDNLQV

SEQ ID NO: 223 - protein, 11 aa, Homo sapiens
  SITE 5..6: note = phosphorylation site
DNFRSSNKYSS

SEQ ID NO: 224 - protein, 12 aa, Homo sapiens
  SITE 5: note = phosphorylation site
  SITE 8: note = phosphorylation site
VGSSSGHTSTTL

SEQ ID NO: 225 - protein, 11 aa, Homo sapiens
  SITE 5..6: note = phosphorylation site
SFVGSSSGHTS

SEQ ID NO: 226 - protein, 10 aa, Homo sapiens
  SITE 5: note = phosphorylation site
DSRPSFVGSS

(19) United States
(12) Patent Application Publication
(10) Pub. No.: US 2025/0003976 A1
(43) Pub. Date: Jan. 2, 2025

(54) ANTIBODIES TARGETING PHOSPHORYLATED SEVEN-TRANSMEMBRANE RECEPTORS

(71) Applicant: 7TM ANTIBODIES GMBH, Jena (DE)

(72) Inventors: Johanna KAUFMANN, Crimmitschau (DE); Stefan SCHULZ, Jena (DE)

(73) Assignee: 7TM ANTIBODIES GMBH, Jena (DE)

(21) Appl. No.: 18/704,515

(22) PCT Filed: Oct. 28, 2022
(86) PCT No.: PCT/EP2022/080265

§ 371 Date: Apr. 25, 2024

(30) Foreign Application Priority Data
Oct. 28, 2021 (EP) 21205185.8
Apr. 12, 2022 (EP) 22167875.8

(51) Int. Cl. G01N 33/68; C07K 16/28; G01N 33/543; G01N 33/573; G01N 33/58

(52) U.S. Cl. CPC G01N 33/68; C07K 16/28; G01N 33/54326; G01N 33/573; G01N 33/582; G01N 2333/912; G01N 2440/14

(57) ABSTRACT

The present invention relates to a collection of seven-transmembrane receptor (7TMR) specific antibodies. A further aspect of the invention relates to a method for determining a phosphorylation status of a 7TMR polypeptide.

This application claims the right of priority of European Patent Applications EP21205185.8 filed 28 Oct. 2021, and EP22167875.8 filed 12 Apr. 2022, both of which are incorporated by reference herein.

### FIELD

The present invention relates to a collection of seven-transmembrane receptor (7TMR) specific antibodies. A further aspect of the invention relates to a method for determining a phosphorylation status of a 7TMR polypeptide.

### BACKGROUND OF THE INVENTION

G protein-coupled receptors (GPCRs) are vital signal transducers that mediate a variety of chemical and physical stimuli. GPCRs have a uniform structure with seven transmembrane domains and are therefore also referred to as 7TM receptors (7TMRs). Activation by their endogenous ligands or exogenous agonists leads to a conformational change that is recognized by a family of kinases called G protein-coupled receptor kinases (GRKs). Thus, the specific ability of GRKs to recognize activated receptors leads to agonist-dependent phosphorylation of GPCRs at serine and threonine residues on intracellular receptor moieties particularly the C-terminus or 3rd intracellular loop. Phosphorylation of GPCRs is a biologically and pharmacologically significant process, which primarily initiates desensitization of the receptor. Furthermore, it also initiates the internalization of GPCRs. In general, phosphorylation increases the receptor's ability to interact with intracellular adapter proteins such as ß-arrestins. It is possible that phosphorylation and subsequent β-arrestin interaction not only mediate desensitization but also trigger a second wave of signalling. Detection of phosphorylation is possible with whole-cell radioactive phosphorylation studies. However, these are laborious because of the need for high levels of radioactivity and do not provide information on which individual serine and threonine sites are phosphorylated. More recently, phosphoproteomic analyses have been increasingly used to determine the agonist-induced phosphorylation sites of individual GPCRs. An alternative method is the use of phosphosite-specific antibodies. However, for targeted generation of such antibodies, the individual serine and threonine sites that are actually the substrates of agonist-dependent phosphorylation must first be identified for the receptor of interest. This regularly requires very time-consuming mutation studies with subsequent analysis in radioactive whole-cell phosphorylation assays as well as further functional assays such as receptor internalization. In the past, very long periods of 10 to 15 years, calculated from the first mutation studies on GPCRs to the successful generation of phosphosite-specific antibodies, were often necessary.

For many applications high throughput assays are needed, which should allow the testing of several thousand substances in a very short time. In contrast to measurements of receptor binding, receptor signaling or arrestin recruitment, such methods for the determination of GPCR phosphorylation are currently NOT available. However, there is a high demand for such methods in academic and industrial research, as evidenced by the high interest and demand for arrestin recruitment measurements (e.g., pathhunter arrestin assay). Phosphorylation fundamentally increases the ability of the receptor to interact with ß-arrestins. However, even alteration of receptor conformation can lead to arrestin recruitment, so arrestin assays can at best be indirect readouts for GPCR phosphorylation. In addition, a large body of the inventors' preliminary work on opioid receptors shows that the determination of agonist-selective phosphorylation patterns can provide substantial new insights for ligand characterization and thus should always be an integral part of the development of novel drugs.

Detection of GPCR phosphorylation using phosphosite-specific antibodies is the favorable method. However, for targeted generation of such antibodies, the individual serine and threonine sites that are actually the substrates of agonist-dependent phosphorylation must first be identified for the receptor of interest. This regularly requires very time-consuming mutation analyses. In the past, very long periods of 10 to 15 years, calculated from the first mutation studies on GPCRs to the successful generation of phosphosite-specific antibodies, were often necessary. Only in a few cases could this work be accomplished by individual research groups so far. To date, this process has also only been successful for about 10 out of 400 GPCRs. However, where available, such phosphosite-specific antibodies represent excellent tools for the detection of receptor activation
the resolution of the temporal and spatial dynamics of receptor phosphorylation
the identification of the kinases and phosphatases involved
the characterization of new ligands.

Due to the versatility of phosphosite-specific antibodies in academic research as well as in the pharmaceutical industry, there is a high demand for such tools as well as for methods to generate and characterize them rapidly and specifically for a variety of receptors.

Up to now, phosphosite-specific antibodies have been predominantly used in Western blotting. This is a very time- and labor-intensive method, which is only routinely performed in a few specialized laboratories and is not suitable for drug screening.

Based on the above-mentioned state of the art, the objective of the present invention is to provide means and methods to provide and use 7TMR-phosphosite-specific antibodies. This objective is attained by the subject-matter of the independent claims of the present specification, with further advantageous embodiments described in the dependent claims, examples, figures and general description of this specification.

### SUMMARY OF THE INVENTION

A first aspect of the invention relates to a collection of seven-transmembrane receptor (7TMR) specific antibodies.

A further aspect of the invention relates to a method for determining a phosphorylation status of a 7TMR polypeptide.

The invention also relates to kits for performing the method.

The inventors have developed and validated a bead-based immunoassay for the determination of agonist-induced phosphorylation of GPCRs in a high-throughput manner. This assay has the following performance characteristics:
1. Determination of GPCR phosphorylation can be performed on cells seeded and treated directly in microtiter plates.
2. The entire assay can then be performed in both 96-well and 384-well formats within 12 to 27 hours.
3. The assay can be performed with GPCRs carrying different affinity tags e.g. HA, FLAG, MYC or HIS.
4. The assay can be performed on both transiently and stably transfected cells.
5. The assay allows both quantitative determination of phosphorylated receptors and measurement of total receptor content.
6. The assay allows the generation of dose-response curves for agonists so that EC₅₀ and Eₘₐₓ values can be determined. (Requirement for Drug Screening)
7. The assay allows the determination of agonist-specific phosphorylation patterns at different phosphorylation sites. (Requirement for Drug Screening)
8. The assay allows the generation of dose-response curves (DWK) for antagonists so that IC₅₀ values can be determined. (Requirement for Drug Screening)
9. The assay allows recording the temporal dynamics of GPCR phosphorylation and dephosphorylation.
10. The assay is suitable for the identification of the kinases and phosphatases involved.
11. The assay allows testing the selectivity and specificity of novel inhibitors of G-protein receptor kinases (GRKs). (Requirement for GRK Inhibitor Screening)
12. This assay allows identification of new ligands for orphan GPCRs.
13. The assay allows initial testing of newly generated phosphorylation site-specific GPCR antibodies.

Due to the versatile application of this assay and the large number of pharmacologically relevant GPCRs, the inventors assume a considerable market potential, so that the application for corresponding property rights is indicated.

### Terms and Definitions

For purposes of interpreting this specification, the following definitions will apply and whenever appropriate, terms used in the singular will also include the plural and vice versa. In the event that any definition set forth below conflicts with any document incorporated herein by reference, the definition set forth shall control.

The terms “comprising,” “having,” “containing,” and “including,” and other similar forms, and grammatical equivalents thereof, as used herein, are intended to be equivalent in meaning and to be open ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, including in the appended claims, the singular forms “a,” “or,” and “the” include plural referents unless the context clearly dictates otherwise.

Unless defined otherwise, all technical and scientific terms used herein have the same meaning as commonly understood by one of ordinary skill in the art (e.g., in cell culture, molecular genetics, nucleic acid chemistry, hybridization techniques and biochemistry). Standard techniques are used for molecular, genetic and biochemical methods (see generally, Sambrook et al., Molecular Cloning: A Laboratory Manual, 4th ed. (2012) Cold Spring Harbor Laboratory Press, Cold Spring Harbor, N.Y. and Ausubel et al., Short Protocols in Molecular Biology (2002) 5th Ed, John Wiley & Sons, Inc.) and chemical methods.

The term 7TMR in the context of the present specification relates to a seven-transmembrane receptor. In certain embodiments, the 7TMR is of the Pfam class PF00001. In certain embodiments, the 7TMR is of the InterPro class IPR000276.

The term GPCR in the context of the present specification relates to a G protein-coupled receptor.

### Sequences

Sequences similar or homologous (e.g., at least about 70% sequence identity) to the sequences disclosed herein are also part of the invention. In some embodiments, the sequence identity at the amino acid level can be about 80%, 85%, 90%, 91%, 92%, 93%, 94%, 95%, 96%, 97%, 98%, 99% or higher. At the nucleic acid level, the sequence identity can be about 70%, 75%, 80%, 85%, 90%, 91%, 92%, 93%, 94%, 95%, 96%, 97%, 98%, 99% or higher. Alternatively, substantial identity exists when the nucleic acid segments will hybridize under selective hybridization conditions (e.g., very high stringency hybridization conditions), to the complement of the strand. The nucleic acids may be present in whole cells, in a cell lysate, or in a partially purified or substantially pure form.

In the context of the present specification, the terms sequence identity and percentage of sequence identity refer to a single quantitative parameter representing the result of a sequence comparison determined by comparing two aligned sequences position by position. Methods for alignment of sequences for comparison are well-known in the art. Alignment of sequences for comparison may be conducted by the local homology algorithm of Smith and Waterman, Adv. Appl. Math. 2:482 (1981), by the global alignment algorithm of Needleman and Wunsch, J. Mol. Biol. 48:443 (1970), by the search for similarity method of Pearson and Lipman, Proc. Nat. Acad. Sci. 85:2444 (1988) or by computerized implementations of these algorithms, including, but not limited to: CLUSTAL, GAP, BESTFIT, BLAST, FASTA and TFASTA. Software for performing BLAST analyses is publicly available, e.g., through the National Center for Biotechnology-Information (http://blast.ncbi.nlm.nih.gov/).

One example for comparison of amino acid sequences is the BLASTP algorithm that uses the default settings: Expect threshold: 10; Word size: 3; Max matches in a query range: 0; Matrix: BLOSUM62; Gap Costs: Existence 11, Extension 1; Compositional adjustments: Conditional compositional score matrix adjustment. One such example for comparison of nucleic acid sequences is the BLASTN algorithm that uses the default settings: Expect threshold: 10; Word size: 28; Max matches in a query range: 0; Match/Mismatch Scores: 1.-2; Gap costs: Linear. Unless stated otherwise, sequence identity values provided herein refer to the value obtained using the BLAST suite of programs (Altschul et al., J. Mol. Biol. 215:403-410 (1990)) using the above identified default parameters for protein and nucleic acid comparison, respectively.

Reference to identical sequences without specification of a percentage value implies 100% identical sequences (i.e. the same sequence).

### General Biochemistry: Peptides, Amino Acid Sequences

The term polypeptide in the context of the present specification relates to a molecule consisting of 50 or more amino acids that form a linear chain wherein the amino acids are connected by peptide bonds. The amino acid sequence of a polypeptide may represent the amino acid sequence of a whole (as found physiologically) protein or fragments thereof. The term “polypeptides” and “protein” are used interchangeably herein and include proteins and fragments thereof. Polypeptides are disclosed herein as amino acid residue sequences.

The term peptide in the context of the present specification relates to a molecule consisting of up to 50 amino acids, in particular 8 to 30 amino acids, more particularly 8 to 15 amino acids, that form a linear chain wherein the amino acids are connected by peptide bonds.

Amino acid residue sequences are given from amino to carboxyl terminus. Capital letters for sequence positions refer to L-amino acids in the one-letter code (Stryer, Biochemistry, 3rd ed. p. 21). Lower case letters for amino acid sequence positions refer to the corresponding D- or (2R)-amino acids. Sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

### Binding; Binders Ligands Antibodies:

The term binder in the context of the present invention refers to a molecule capable of specifically binding to a target moiety as specified herein.

The term specific binding in the context of the present invention refers to a property of ligands that bind to their target with a certain affinity and target specificity. The affinity of such a ligand is indicated by the dissociation constant of the ligand. A specifically reactive ligand has a dissociation constant of ≤10⁻⁷ mol/L when binding to its target, but a dissociation constant at least three orders of magnitude higher in its interaction with a molecule having a globally similar chemical composition as the target, but a different three-dimensional structure.

The term affinity tag in the context of the present invention refers to a moiety which is specifically bound by a binder. In certain embodiments, the affinity tag is a peptide. A recombinantly expressed protein may comprise an affinity tag at its C-terminal or N-terminal end. The affinity tag may be used to purify the protein or quantify the amount of protein. Various affinity tags are known in the art, as for example, but not limited to, HA-tag, FLAG-tag, GFP-tag, Myc-tag, His-tag, Strep-tag, T7-tag, and V5-tag.

In the context of the present specification, the term antibody refers to whole antibodies including but not limited to immunoglobulin type G (IgG), type A (IgA), type D (IgD), type E (IgE) or type M (IgM), any antigen binding fragment or single chains thereof and related or derived constructs. A whole antibody is a glycoprotein comprising at least two heavy (H) chains and two light (L) chains inter-connected by disulfide bonds. Each heavy chain is comprised of a heavy chain variable region (VH) and a heavy chain constant region (C_{H}). The heavy chain constant region of IgG is comprised of three domains, C_{H}1, C_{H}2 and C_{H}3. Each light chain is comprised of a light chain variable region (abbreviated herein as V_{L}) and a light chain constant region (C_{L}). The light chain constant region is comprised of one domain, C_{L}. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component of the classical complement system. Similarly, the term encompasses a so-called nanobody or single domain antibody, an antibody fragment consisting of a single monomeric variable antibody domain.

A first aspect of the invention relates to a collection of seven-transmembrane receptor (7TMR) specific antibodies, the collection comprising a plurality of members, wherein
each member of said collection of antibodies is capable of specifically binding to a target sequence comprised in a 7TMR polypeptide, said target sequence comprising phosphorylation sites,
each member is
   i. capable of
      specifically binding to its target sequence if the target sequence is phosphorylated, wherein the target sequence has 1, 2, or 3 phosphorylation sites, and wherein the target sequence has any possible phosphorylation pattern, and the target sequence is selected from a group of sequences comprising SEQ ID NO 1-51 and SEQ ID NO 139-141 and SEQ ID NO 145-178,
      particularly wherein the target sequence is phosphorylated in all phosphorylation sites (phosphorylation sites are indicated as phosphorylated or phosphorylatable in the sequence protocol, and underlined in Table 1);
      or
      specifically binding to its target sequence if the target sequence is phosphorylated in all indicated phosphorylation sites, and the target sequence is selected from a group of sequences comprising SEQ ID NO 52-54 and SEQ ID NO 179-182,
   ii. and not capable of specifically binding to the corresponding unphosphorylated target sequence;
wherein the collection comprises at least 2, particularly at least 3, 4, 5, 6, 7, 8, 9, or 10 members, each capable of specifically binding to a phosphorylated target sequence.

In certain embodiments, the 7TMR is a GPCR.

In certain embodiments, the collection additionally comprises at least one additional member, wherein
each additional member of said collection of antibodies is capable of specifically binding to a target sequence comprised in a 7TMR polypeptide, said target sequence comprising phosphorylation sites,
each additional member is
   i. capable of
      specifically binding to its target sequence if the target sequence is phosphorylated, wherein the target sequence has 1, 2, or 3 phosphorylation sites, and wherein the target sequence has any possible phosphorylation pattern, and the target sequence is selected from a group of sequences comprising SEQ ID NO 55-138 and SEQ ID NO 142-144 and SEQ ID NO 183-226, particularly wherein the target sequence is phosphorylated in all phosphorylation sites (phosphorylation sites are indicated as phosphorylated or phosphorylatable in the sequence protocol, and underlined in Table 1);
   ii. and not capable of specifically binding to the corresponding unphosphorylated target sequence;
      particularly the collection comprises at least 2, more particularly at least 3, 4, 5, 6, 7, 8, 9, or 10 additional members.

In certain embodiments, each member specifically binding to a different phosphorylated sequence is a polyclonal antibody. A polyclonal antibody comprises a plurality of antibody molecules which were obtained by immunizing an animal with the corresponding target sequence. In certain embodiments, each member specifically binding to a different phosphorylated sequence is a monoclonal antibody.

A second aspect of the invention relates to a preparation of an antibody, wherein
the antibody is capable of specifically binding to a target sequence comprised in a 7TMR polypeptide, said target sequence comprising phosphorylation sites,
the antibody is
   i. capable of
      specifically binding to its target sequence if the target sequence is phosphorylated, wherein the target sequence has 1, 2, or 3 phosphorylation sites, and wherein the target sequence has any possible phosphorylation pattern, and the target sequence is selected from a group of sequences comprising SEQ ID NO 1-51 and SEQ ID NO 139-141 and SEQ ID NO 145-178,
      particularly wherein the target sequence is phosphorylated in all phosphorylation sites (phosphorylation sites are indicated as phosphorylated or phosphorylatable in the sequence protocol, and underlined in Table 1);
      or
      specifically binding to its target sequence if the target sequence is phosphorylated in all indicated phosphorylation sites, and the target sequence is selected from a group of sequences comprising SEQ ID NO 52-54 and SEQ ID NO 179-182,
   ii. and not capable of specifically binding to the corresponding unphosphorylated target sequence.

An alternative of the second aspect of the invention relates to a preparation of an antibody, wherein
the antibody is capable of specifically binding to a target sequence comprised in a 7TMR polypeptide, said target sequence comprising phosphorylation sites,
the antibody is
   i. capable of
      specifically binding to its target sequence if the target sequence is phosphorylated, wherein the target sequence has 1, 2, or 3 phosphorylation sites, and wherein the target sequence has any possible phosphorylation pattern, and the target sequence is selected from a group of sequences comprising SEQ ID NO 188-221,
      particularly wherein the target sequence is phosphorylated in all phosphorylation sites (phosphorylation sites are indicated as phosphorylated or phosphorylatable in the sequence protocol, and underlined in Table 1);
   ii. and not capable of specifically binding to the corresponding unphosphorylated target sequence.

A third aspect of the invention relates to a kit comprising the collection of antibodies according to the first aspect and its embodiments.

A fourth aspect of the invention relates to a method for determining a phosphorylation status of a 7TMR polypeptide, said method comprising the steps:
providing a cell comprising a recombinant 7TMR construct, wherein the 7TMR construct comprises a 7TMR polypeptide having a phosphorylation status of interest, and an affinity tag;
in a lysis step, lysing the cell with a detergent-containing lysis buffer yielding a cell lysate;
in a first contacting step, contacting the cell lysate with magnetic beads coated with a first binder;
washing off unbound cell lysate from the magnetic beads via rinsing the magnetic beads with washing buffer under influence of magnetic force;
in a second contacting step, contacting the magnetic beads with a second binder;
wherein one of the first and the second binder is capable of binding to the affinity tag, and the other one of the first and the second binder is a first antibody capable of specifically binding to said 7TMR polypeptide when said 7TMR polypeptide is in a phosphorylated status, but not when said 7TMR polypeptide is in an unphosphorylated status; and
wherein the second binder is coupled directly or indirectly to a detection moiety;
washing off unbound secondary antibody from the magnetic beads via rinsing the magnetic beads with washing buffer under influence of magnetic force;
in a detection step, detecting the phosphorylation status of the 7TMR polypeptide by measuring a signal derived from the detection moiety (the signal amplitude/volume being dependent/linearly correlated to the amount of detection moiety).

An indirect coupling means that the magnetic beads are contacted with a third binder, wherein the third binder specifically binds to the second binder, and the third binder is coupled to a detection moiety.

Magnetic beads, also called magnetic particles, are small spheres of a diameter of ˜1 μm. In certain embodiments, the magnetic beads are superparamagnetic. This means that they do not have a magnetic memory.

In certain embodiments, the 7TMR is a GPCR.

A detection moiety is a moiety that is capable of producing a measurable signal. The detection moiety may be selected from for example, but not limited to, a fluorophore, an enzyme, a metal, an isotope, and a dye. The signal derived from the detection moiety may be for example, but not limited to, emitted light of a fluorophore, enzymatic production of a dye, radiation of an isotope, or visual or mass-based detection of a metal or a dye.

In certain embodiments, the lysis buffer comprises an inhibitor of phosphatases and/or an inhibitor of proteases.

In certain embodiments, after the lysis step, the cell lysate is centrifuged, and the supernatant is used in the contact step.

In certain embodiments, the method is performed in a 96-well plate or a 384-well plate.

In certain embodiments, the cell is grown prior to the lysis step inside the 96-well plate or 384-well plate.

In certain embodiments, the detection moiety is selected from a fluorophore, and an enzyme catalyzing a reaction producing a dye. In certain embodiments, the enzyme is selected from horse-radish peroxidase (HRP) and alkaline phosphatase (AP).

In certain embodiments, the magnetic beads carry an isotope or a fluorescent label. In certain embodiments, the magnetic beads carry a fluorescent label.

In certain embodiments, the magnetic beads are coupled to a plurality of first antibodies, and each first antibody of said plurality specifically is capable of binding to a different phosphorylated sequence and is bound to magnetic beads carrying a label characterized by a different isotope or a different fluorescent color.

In certain embodiments, the cell lysate is split into a first half and a second half after the lysis step, and wherein
the first half is contacted with said first antibody, and
the second half is contacted with a binder for the 7TMR, wherein the binder for the 7TMR does not depend on a phosphorylation status of the 7TMR,
   and wherein a ratio between a signal of the first antibody and a signal of the binder for the 7TMR is indicative of an amount of phosphorylation of the 7TMR.

In certain embodiments, the 7TMR construct comprises a first affinity tag and a second affinity tag, and wherein
a binder of the first affinity tag is coupled to the magnetic beads, and
a binder of the second affinity tag is coupled directly or indirectly to a detection moiety,
and wherein a ratio between a signal of the first antibody and a signal of the binder of the second affinity tag is indicative of an amount of phosphorylation of the 7TMR.

A fifth aspect of the invention relates to a method for determining a phosphorylation status of a 7TMR polypeptide, the method comprising the steps:
providing a cell comprising a recombinant 7TMR construct, wherein the 7TMR construct comprises a 7TMR polypeptide having a phosphorylation status of interest, and an affinity tag;
in a lysis step, lysing the cell with a lysis buffer yielding a cell lysate;
in a binding step, contacting the sample with
i. an affinity binder; and
ii. a first antibody being capable of specifically binding to said 7TMR polypeptide in phosphorylated status, but not in unphosphorylated status;
wherein one of the affinity binders and the first antibody is coupled to an emission moiety, and the other one of the binders and the antibody is coupled to an excitation moiety, wherein the emission moiety is capable of emitting light at a wavelength which corresponds to the excitation wavelength of the excitation moiety;
in a detection step, detecting the phosphorylation status of the 7TMR polypeptide via measuring a signal derived from the excitation moiety, optionally exciting the emission moiety with light of an appropriate wavelength.

In certain embodiments, the emission moiety and the excitation moiety are a FRET (Förster resonance energy transfer) pair. The emission moiety is excited with an appropriate wavelength, and emits light at another wavelength. This emitting wavelength is the wavelength, where the excitation moiety is excited. If the emission moiety and the excitation moiety are close enough, the excitation moiety will emit light, when the emission moiety is excited.

In certain embodiments, the emission moiety and the excitation moiety are a BRET (bioluminescence resonance energy transfer) pair, wherein the emission moiety is an enzyme being capable of catalyzing a reaction emitting light under the conditions employed. If the emission moiety and the excitation moiety are close enough, the excitation moiety will emit light due to its excitation by the emission moiety.

A sixth aspect of the invention relates to a method for determining an activity of a 7TMR ligand of interest, said method comprising the steps:
in a ligand step, contacting a cell comprising a 7TMR polypeptide with a 7TMR ligand of interest;
determining the phosphorylation status of said 7TMR polypeptide via the method of the fourth or the fifth aspect.

A seventh aspect of the invention relates to a method for identifying a kinase specific for a distinct 7TMR phosphorylation pattern, wherein a cell comprises a plurality of kinases specific for a 7TMR polypeptide, the method comprising the steps:
providing a plurality of cells, wherein each cell of said plurality comprises only one kinase of said plurality of kinases, and all other kinases are knocked-out;
performing the method of the fourth or the fifth aspect on all cells of said plurality;
determining which kinase of said plurality is specific for said distinct 7TMR phosphorylation pattern.

An alternative aspect relates to a method for identifying a ligand activating an orphan 7TMR polypeptide, the method comprising the steps of the method of the fourth or the fifth aspect performed with an orphan 7TMR polypeptide.

An orphan receptor is a protein that has a similar structure to other identified receptors but whose endogenous ligand has not yet been identified.

In certain embodiments, the plurality of cells is contacted with a kinase inhibitor of interest prior to the lysis step.

An eighth aspect of the invention relates to a method for identifying a 7TMR polypeptide activated by a ligand of interest, said method comprising the steps of the method of the seventh aspect being repetitively executed for a plurality of 7TMR polypeptides.

A nineth aspect of the invention relates to a kit for performing the method according to the fourth aspect and its embodiments comprising
an antibody specifically binding to a 7TMR polypeptide in phosphorylated status, but not in unphosphorylated status;
a binder capable of binding to an affinity tag;
magnetic beads coated with a moiety which can be coupled to the antibody or the binder, wherein the magnetic beads optionally carry a label selected from a fluorescent label, and an isotope label.

An alternative of the nineth aspect relates to a kit for performing the method according to the fifth aspect and its embodiments comprising
an antibody specifically binding to a 7TMR polypeptide in phosphorylated status, but not in unphosphorylated status;
a binder capable of binding to an affinity tag,
wherein one of the binder and the antibody is coupled to an emission moiety, and the other one of the binder and the antibody is coupled to an excitation moiety, wherein the emission moiety is capable of emitting light at a wavelength which corresponds to the excitation wavelength of the excitation moiety.

A tenth aspect of the invention relates to a method for identifying a modulator, particularly an inhibitor, of a kinase specific for a distinct 7TMR phosphorylation pattern, said method comprising the steps
providing a cell comprising a 7TMR construct, wherein the 7TMR construct comprises a 7TMR polypeptide, and an affinity tag;
contacting said cell with a compound of interest;
performing the method of the fourth or the fifth aspect on said cell;
determining whether said compound of interest has an effect on a phosphorylation pattern of said 7TMR polypeptide, and thereby identifying a modulator of a kinase specific for a distinct 7TMR phosphorylation pattern.

In certain embodiments, the ligand of interest is an agonist. In certain embodiments, the ligand is an antagonist. In certain embodiments, the first antibody
i. is capable of specifically binding to
   a. a target sequence if the target sequence is phosphorylated and the target sequence is selected from a group of sequences comprising SEQ ID NO 1-51 and SEQ ID NO 139-141 and SEQ ID NO 145-178, particularly wherein the target sequence is phosphorylated in all phosphorylation sites,
      or
   b. a target sequence if the target sequence is phosphorylated in all indicated phosphorylation sites and the target sequence is selected from a group of sequences comprising SEQ ID NO 52-54 and SEQ ID NO 179-182,
ii. and is not capable of binding to the corresponding unphosphorylated sequence.

In certain embodiments, the first antibody
i. is capable of specifically binding to
   a. a target sequence if the target sequence is phosphorylated and the target sequence is selected from a group of sequences comprising SEQ ID NO 1-226,
      particularly wherein the target sequence is phosphorylated in all phosphorylation sites,
ii. and is not capable of binding to the corresponding unphosphorylated sequence.

Wherever alternatives for single separable features such as, for example, an isotype protein or a method type are laid out herein as “embodiments”, it is to be understood that such alternatives may be combined freely to form discrete embodiments of the invention disclosed herein. Thus, any of the alternative embodiments for an isotype protein may be combined with any of the alternative embodiments of a method type mentioned herein.

The application further encompasses the following items:

### ITEMS

1. A collection of antibodies, the collection comprising a plurality of members, wherein
   each member of said collection of antibodies is capable of specifically binding to a target sequence comprised in a 7TMR polypeptide, said target sequence comprising phosphorylation sites,
   each member is
      i. capable of
         specifically binding to its target sequence if the target sequence is phosphorylated, and the target sequence is selected from a group of sequences comprising SEQ ID NO 1-51 and SEQ ID NO 139-141 and SEQ ID NO 145-178,
         particularly wherein the target sequence is phosphorylated in all phosphorylation sites;
         or
         specifically binding to its target sequence if the target sequence is phosphorylated in all indicated phosphorylation sites, and the target sequence is selected from a group of sequences comprising SEQ ID NO 52-54 and SEQ ID NO 179-182,
      ii. and not capable of specifically binding to the corresponding unphosphorylated target sequence;
   wherein the collection comprises at least 2, particularly at least 3, 4, 5, 6, 7, 8, 9, or 10 members.
2. The collection of antibodies according to item 1, wherein the collection additionally comprises at least one additional member, wherein
   each additional member of said collection of antibodies is capable of specifically binding to a target sequence comprised in a 7TMR polypeptide, said target sequence comprising phosphorylation sites,
   each additional member is
      i. capable of
         specifically binding to its target sequence if the target sequence is phosphorylated, and the target sequence is selected from a group of sequences comprising SEQ ID NO 55-138 and SEQ ID NO 142-144 and SEQ ID NO 183-226, particularly wherein the target sequence is phosphorylated in all phosphorylation sites;
      ii. and not capable of specifically binding to the corresponding unphosphorylated target sequence;
   particularly the collection comprises at least 2, more particularly at least 3, 4, 5, 6, 7, 8, 9, or 10 additional members.
3. The collection of antibodies according to item 1 or 2, wherein each member is a polyclonal antibody.
4. A preparation of an antibody, wherein
   the antibody is capable of specifically binding to a target sequence comprised in a 7TMR polypeptide, said target sequence comprising phosphorylation sites,
   the antibody is
      i. capable of.
         specifically binding to its target sequence if the target sequence is phosphorylated, and the target sequence is selected from a group of sequences comprising SEQ ID NO 1-51 and SEQ ID NO 139-141 and SEQ ID NO 145-178,
         particularly wherein the target sequence is phosphorylated in all phosphorylation sites;
         or
         specifically binding to its target sequence if the target sequence is phosphorylated in all indicated phosphorylation sites, and the target sequence is selected from a group of sequences comprising SEQ ID NO 52-54 and SEQ ID NO 179-182,
      ii. and not capable of specifically binding to the corresponding unphosphorylated target sequence.
5. A preparation of an antibody, wherein
   the antibody is capable of specifically binding to a target sequence comprised in a 7TMR polypeptide, said target sequence comprising phosphorylation sites,
   the antibody is
      i. capable of.
         specifically binding to its target sequence if the target sequence is phosphorylated, and the target sequence is selected from a group of sequences comprising SEQ ID NO 188-221,
         particularly wherein the target sequence is phosphorylated in all phosphorylation sites;
      ii. and not capable of specifically binding to the corresponding unphosphorylated target sequence.
6. A method for determining a phosphorylation status of a 7TMR polypeptide, said method comprising the steps:
   providing a cell comprising a 7TMR construct, wherein the 7TMR construct comprises a 7TMR polypeptide, and an affinity tag;
   in a lysis step, lysing the cell with a lysis buffer yielding a cell lysate;
   in a first contacting step, contacting the cell lysate with magnetic beads coated with a first binder;
   in a second contacting step, contacting the magnetic beads with a second binder;
   wherein one of the first and the second binder is capable of binding to the affinity tag, and the other one of the first and the second binder is a first antibody specifically binding to said 7TMR polypeptide in phosphorylated status, but not in unphosphorylated status; and
   wherein the second binder is coupled directly or indirectly to a detection moiety;
   in a detection step, detecting the phosphorylation status of the 7TMR polypeptide via measuring a signal derived from the detection moiety.
7. The method according to item 6, wherein the lysis buffer comprises an inhibitor of phosphatases and/or an inhibitor of proteases.
8. The method according to any one of items 6 or 7, wherein after the lysis step, the cell lysate is centrifuged, and the supernatant is used in the contact step.
9. The method according to any one of items 6 to 8, wherein the method is performed in a 96-well plate or a 384-well plate.
10. The method according to item 9, wherein the cell is grown prior to the lysis step inside the 96-well plate or 384-well plate.
11. The method according to any one of items 6 to 10, wherein the detection moiety is selected from a fluorophore, and an enzyme catalyzing a reaction producing a dye, particularly an enzyme selected from horse-radish peroxidase (HRP) and alkaline phosphatase (AP).
12. The method according to any one of items 6 to 11, wherein the magnetic beads carry an isotope or a fluorescent label, more particularly a fluorescent label.
13. The method according to item 12, wherein the magnetic beads are coupled to a plurality of first antibodies, and each first antibody of said plurality specifically binds to a different phosphorylated sequence and is bound to magnetic beads carrying a label characterized by a different isotope or a different fluorescent color.
14. The method according to any one of items 6 to 13, wherein the cell lysate is split into a first half and a second half after the lysis step, and wherein
   the first half is contacted with said first antibody, and
   the second half is contacted with a binder for the 7TMR, wherein the binder for the 7TMR does not depend on a phosphorylation status of the 7TMR,
   and wherein a ratio between a signal of the first antibody and a signal of the binder for the 7TMR is indicative of an amount of phosphorylation of the 7TMR.
15. The method according to any one of items 6 to 13, wherein the 7TMR construct comprises a first affinity tag and a second affinity tag, and wherein
   a binder of the first affinity tag is coupled to the magnetic beads, and
   a binder of the second affinity tag is coupled directly or indirectly to a detection moiety,
   and wherein a ratio between a signal of the first antibody and a signal of the binder of the second affinity tag is indicative of an amount of phosphorylation of the 7TMR.
16. A method for determining a phosphorylation status of a 7TMR polypeptide, said method comprising the steps:
   providing a cell comprising a 7TMR construct, wherein the 7TMR construct comprises a 7TMR polypeptide, and an affinity tag;
   in a lysis step, lysing the cell with a lysis buffer yielding a cell lysate;
   in a binding step, contacting the sample with
      i. an affinity binder; and
      ii. a first antibody specifically binding to said 7TMR polypeptide in phosphorylated status, but not in unphosphorylated status;
   wherein one of the affinity binder and the first antibody is coupled to an emission moiety, and the other one of the binder and the antibody is coupled to an excitation moiety, wherein the emission moiety is capable of emitting light at a wavelength which corresponds to the excitation wavelength of the excitation moiety;
   in a detection step, detecting the phosphorylation status of the 7TMR polypeptide via measuring a signal derived from the excitation moiety, optionally exciting the emission moiety with light of an appropriate wavelength.
17. The method according to item 16, wherein the emission moiety and the excitation moiety are a FRET (Förster resonance energy transfer) pair.
18. The method according to item 16, wherein the emission moiety and the excitation moiety are a BRET (bioluminescence resonance energy transfer) pair.
19. A method for determining an activity of a 7TMR ligand of interest, said method comprising the steps:
   in a ligand step, contacting a cell comprising a 7TMR polypeptide with a 7TMR ligand of interest;
   determining the phosphorylation status of said 7TMR polypeptide via the method of any one of items 6 to 18.
20. A method for identifying a kinase specific for a distinct 7TMR phosphorylation pattern, wherein a cell comprises a plurality of kinases specific for a 7TMR polypeptide, the method comprising the steps:
   providing a plurality of cells, wherein each cell of said plurality comprises only one kinase of said plurality of kinases;
   performing the method of items 6 to 18 on all cells of said plurality;
   determining which kinase of said plurality is specific for said distinct 7TMR phosphorylation pattern.
21. The method according to item 20, wherein the plurality of cells is contacted with a kinase inhibitor of interest prior to the lysis step.
22. A method for identifying a 7TMR polypeptide activated by a ligand of interest, said method comprising the steps of the method of item 19 being repetitively executed for a plurality of 7TMR polypeptides.
23. The method according to any one of items 6 to 22, wherein the first antibody
   i. specifically binds to
      a. a target sequence if the target sequence is phosphorylated and the target sequence is selected from a group of sequences comprising SEQ ID NO 1-51 and SEQ ID NO 139-141 and SEQ ID NO 145-178, particularly wherein the target sequence is phosphorylated in all phosphorylation sites,
      or
      b. a target sequence if the target sequence is phosphorylated in all indicated phosphorylation sites and the target sequence is selected from a group of sequences comprising SEQ ID NO 52-54 and SEQ ID NO 179-182,
   ii. and does not bind to the corresponding unphosphorylated sequence.
24. A method for identifying a modulator, particularly an inhibitor, of a kinase specific for a distinct 7TMR phosphorylation pattern, said method comprising the steps
   providing a cell comprising a 7TMR construct, wherein the 7TMR construct comprises a 7TMR polypeptide, and an affinity tag;
   contacting said cell with a compound of interest;
   performing the method of items 6 to 18 on said cell;
   determining whether said compound of interest has an effect on a phosphorylation pattern of said 7TMR polypeptide, and thereby identifying a modulator of a kinase specific for a distinct 7TMR phosphorylation pattern.
25. A kit for performing the method according to any one of items 6 to 13 comprising
   an antibody specifically binding to a 7TMR polypeptide in phosphorylated status, but not in unphosphorylated status;
   a binder capable of binding to an affinity tag;
   magnetic beads coated with a moiety which can be coupled to the antibody or the binder, wherein the magnetic beads optionally carry a label selected from a fluorescent label, and an isotope label.
26. A kit for performing the method according to any one of items 16 to 18 comprising
   an antibody specifically binding to a 7TMR polypeptide in phosphorylated status, but not in unphosphorylated status;
   a binder capable of binding to an affinity tag,
   wherein one of the binder and the antibody is coupled to an emission moiety, and the other one of the binder and the antibody is coupled to an excitation moiety, wherein the emission moiety is capable of emitting light at a wavelength which corresponds to the excitation wavelength of the excitation moiety.
27. A kit comprising the collection of antibodies according to any one of items 1 to 3.

The invention is further illustrated by the following examples and figures, from which further embodiments and advantages can be drawn. These examples are meant to illustrate the invention but not to limit its scope.

### DESCRIPTION OF THE FIGURES

FIG. 1 shows Step-by-step flowchart showing the 7TM phosphorylation assay protocol. (1) Cells expressing affinity-tagged GPCRs are grown in F-bottom cell culture plates, and upon reaching ≥95% confluency, the cells are exposed to the agonist, antagonist or inhibitor of interest. (2) The cells are lysed in detergent in buffer, and lysates are cleared by centrifugation. (3) For parallel detection of phosphorylated and total receptors, the lysate of each sample is divided into corresponding wells in U-bottom assay plates. (4) Anti-tag magnetic beads are added to each well for receptor immunoprecipitation. (5) Primary phosphosite-specific and phosphorylation-independent antibodies are added to the appropriate wells of each split sample. (6) A secondary antibody labeled with an enzyme or other detection entity is then added. (7) An enzyme substrate solution is added for detection, the color reaction is stopped by adding a stop solution, and the optical density (OD) is determined with a microplate reader. (Created with BioRender.com)

FIG. 2 shows a suggested experimental layout for antagonist stimulation according to the method.

FIG. 3 shows a first selection of western-blots. The respective SEQ ID NO: is indicated for each of the sequences.

FIG. 4 shows absorbance of OD at 405 nm of phosphosite-specific antibodies at MOP.

FIG. 5 shows a further selection of western-blots of a selection of phosphorylated sequences. The SEQ ID NO. is given for each of the phosphorylated sequences.

FIG. 6 shows development and validation of the 7TM phosphorylation assay. In all experiments, the mouse μ-opioid receptor (MOP) was stimulated with DAMGO for 30 min at 37° C. (a) Schematic representation showing MOP with anti-hemagglutinin (HA) magnetic bead-binding sites, anti-phosphorylated (p) S375-MOP antibody that selectively detects pS375-MOP and an antibody that detects MOP independent of phosphorylation status (np-MOP). (b) Comparison of optical density (OD) values under different assay conditions: stimulated (+) or unstimulated (−), no primary antibody, no secondary antibody, no beads with MOP-transfected or untransfected (WT)-HEK293 cells. An anti-pS375-MOP was used as the primary antibody in all conditions except for the np-MOP samples. (c) The concentration-response OD readings with increasing DAMGO concentrations were determined with anti-pS375-MOP and anti-np-MOP antibodies. (d-f) MOP-HEK293 cells were treated with increasing DAMGO concentrations. Cells were lysed in detergent buffer in the presence or absence of protein phosphatase inhibitors (+/−PPase-I) and either analyzed by Western blotting (d), pS375-MOP assay (f) or np-MOP phosphorylation assay (e). (g) For peptide neutralization, the anti-pS375-MOP antibody was preincubated with 1 μg/ml of pS375 peptide (solid blue line) or the corresponding unphosphorylated peptide (dashed blue line) for 1 h before the addition of each antibody corresponding to the beads. Control samples (solid red line) were prepared according to a standard assay protocol. (h) Comparison of DAMGO concentration-response curves after the addition of different amounts of anti-HA magnetic beads, ranging from 1-50 μg per well. The optimal amount is depicted as a red solid line. (i) Comparison of DAMGO concentration-response curves after the addition of different amounts of primary antibody, ranging from 0.5-8 μg/ml. The optimal amount is depicted as a red solid line. (j-l) For determination of detection limits, lysates from DAMGO-stimulated MOP-HEK293 and WT-HEK293 cells were combined at different ratios to yield a final volume of 100 μl and analyzed either by Western blotting (j), pS375-MOP phosphorylation assay (k) or np-MOP phosphorylation assay (l). Mean of all assay data points were calculated from n=4-5 independent experiments performed in duplicates±SEM. Western Blot images are representatives of at least n=3 replicates.

FIG. 7 shows profiling of μ-opioid receptor (MOP) agonists and antagonists using the 7TM phosphorylation assay. (a) Schematic representation showing the MOP binding sites for antibodies against phosphorylated (p) T370-, pS375-, pT376-, and pT379-MOP and MOP independent of phosphorylation (np-MOP). (b-f) Concentration-response curves were generated after treatment with DAMGO (b), naloxone (c), morphine (d), fentanyl (e) and the PKC activator phorbol 12-myristate 13-acetate (PMA) (f). The cells were exposed to agonists or PMA for 30 min at 37° C. The antagonist naloxone was added 30 min prior to DAMGO stimulation. The cells were then lysed and analyzed according to the standard assay protocol. Every graph represents the mean of n=5 independent experiments performed in duplicates±SEM. All data points were normalized to 10 μM DAMGO stimulation.

FIG. 8 shows Differential involvement of GRK2/3 and GRK5/6 in C5a1 multisite phosphorylation. (a-d) Control-HEK293 cells (gray), ΔGRK2/3-HEK293 cells (orange) and ΔGRK5/6-HEK293 cells (indigo) stably expressing C5a1 were exposed to increasing concentrations of the synthetic agonist C028 and incubated for 30 min at 37° C. Phosphorylation of T324/S327 (a), S332/S334 (b), S338/T339 (c) and T342 (d) was assessed according to the standard protocol. Means of n=5 independent experiments performed in duplicates±SEM are presented in concentration-response curves. All data points were normalized to 10 UM C028 stimulation of C5a1-expressing Control-HEK293 cells.

FIG. 9 shows GRK inhibitor screening using the 7TM phosphorylation assay. (a) Control-HEK293, ΔGRK2/3-HEK293, ΔGRK5/6-HEK293 and ΔGRK2/3/5/6-HEK293 cells stably expressing mouse μ-opioid receptor (MOP) were either unstimulated (−) or exposed to 10 μM DAMGO (+) for 30 min at 37° C. Subsequently, T370, S375, T376 and T379 phosphorylation was determined by Western blot analysis. (b) MOP-expressing Control-HEK293 and ΔGRK2/3-HEK293 cells were exposed to increasing concentrations of DAMGO, and T376 phosphorylation was determined according to the standard protocol. (c) MOP-expressing Control-HEK293 and ΔGRK2/3-HEK293 cells were exposed to increasing concentrations of DAMGO, and the degree of S375 phosphorylation was determined. (d) MOP-expressing Control-HEK293 cells were treated with increasing concentrations of the GRK inhibitors LDC9728, LDC8988 or compound 101 for 30 min prior to stimulation with 10 μM DAMGO, and T376 phosphorylation was then determined. (e) MOP-expressing ΔGRK2/3-HEK293 cells were treated with increasing concentrations of the GRK inhibitors LDC9728, LDC8988 or compound 101 for 30 min prior to stimulation with 10 UM DAMGO, and S375 phosphorylation was then determined. The data points shown in (b and c) represent optical density read at 405 nm (OD₄₀₅). Data points shown in (b and c) represent optical density readings at 405 nm (OD₄₀₅). Data points shown in (d) were normalized to 10 UM DAMGO stimulation of MOP-expressing Control-HEK293 cells. The data points shown in (e) were normalized to 10 UM DAMGO stimulation of MOP-expressing ΔGRK2/3-HEK293 cells. Western Blot images are representatives of n=5 independent experiments. Immunoassay data are means±SEM of n=5 independent experiments performed in quadruplicates.

FIG. 10 shows OD signals in C5a1 phosphorylation assays. (a) Schematic representation of C5a1 depicting antibody binding sites for pT324/pS327-, pS332/pS334-, pS338/pT339-, pT342- and np-C5a1. (b-c) C5a1-HEK293 cells were treated with increasing C028 concentrations. Phosphorylation was determined using pT324/pS327-C5a1 (b), pS332/pS334-C5a1 (c), pS338/pT339-C5a1 (d) and pT342-C5a1 (e) antibodies according to 7TM phosphorylation assay protocol. All data points represent means of n=5 independent experiments performed in duplicates±SEM.

FIG. 11 shows OD signals in β2 adrenoceptor phosphorylation assays. (a) Schematic representation of β2 adrenergic receptor depicting antibody binding sites for pS355/pS356-, pT360/pS364- and np-B2 in the third intracellular loop (3ICL). (b, c) β2-HEK293 cells were treated with increasing isoprenaline concentrations. Phosphorylation was determined using pS355/pS356-β2 (b) and pT360/pS364-β2 (c) antibodies according to 7TM phosphorylation assay protocol. All data points represent means of n=5 independent experiments performed in duplicates±SEM.

FIG. 12 shows OD signals in SST2 somatostatin receptor phosphorylation assays. (a) Schematic representation of SST2 receptor depicting antibody binding sites for pS341/pS343- and pT356/pT359-SST2. (b, c) SST2-HEK293 cells were treated with increasing SRIF concentrations. Phosphorylation was determined using pS341/pS343-SST2 (b) and pT356/pT359-SST2 (c) antibodies according to 7TM phosphorylation assay protocol. All data points represent means of n=4 independent experiments performed in duplicates±SEM.

FIG. 13 shows comparison Assay-efficacy. The top shows the drawbacks of a Direct ELISA assay. The middle shows the drawbacks of Capture ELISA. The bottom shows Bead-Based ELISA as object of the invention.

FIG. 14 shows OD (405 nm) signals in C5aR1 complement receptor phosphorylation assays. Phosphorylation was determined by using the respective antibodies (plot legend) according to 7TM phosphorylation protocol.

FIG. 15 shows shows OD (405 nm) signals in CB2 cannabinoid receptor phosphorylation assays. Phosphorylation was determined by using the respective antibodies (plot legend) according to 7TM phosphorylation protocol.

FIG. 16 shows OD (405 nm) signals in D1 dopamine receptor phosphorylation assays. Phosphorylation was determined by using the respective antibodies (plot legend) according to 7TM phosphorylation protocol.

FIG. 17 shows OD (405 nm) signals in MOP MU-Opiod receptor phosphorylation assays. Phosphorylation was determined by using the respective antibodies (plot legend) according to 7TM phosphorylation protocol.

FIG. 18 shows OD (405 nm) signals in NK1 Neurokinin receptor phosphorylation assays. Phosphorylation was determined by using the respective antibodies (plot legend) according to 7TM phosphorylation protocol.

FIG. 19 shows OD (405 nm) signals in NPY2 Neuropeptide Y receptor phosphorylation assays. Phosphorylation was determined by using the respective antibodies (plot legend) according to 7TM phosphorylation protocol.

FIG. 20 shows OD (405 nm) signals in PAC1 PACAP receptor phosphorylation assays. Phosphorylation was determined by using the respective antibodies (plot legend) according to 7TM phosphorylation protocol.

FIG. 21 shows OD (405 nm) signals in B2 Adrenoceptor receptor phosphorylation assays. Phosphorylation was determined by using the respective antibodies (plot legend) according to 7TM phosphorylation protocol.

FIG. 22 shows OD (405 nm) signals in SST2 Somatostatin receptor phosphorylation assays. Phosphorylation was determined by using the respective antibodies (plot legend) according to 7TM phosphorylation protocol.

FIG. 23 shows OD (405 nm) signals in TRH1 receptor phosphorylation assays. Phosphorylation was determined by using the respective antibodies (plot legend) according to 7TM phosphorylation protocol.

FIG. 24 shows a second selection of western-blots. The respective SEQ ID NO. is indicated for each of the sequences.

### EXAMPLES

### Example 1

Microplate-based immunoassays often use an enzyme activity, e.g. peroxidase or alkaline phosphatase, as the detection reaction and are therefore also referred to as “enzyme-linked immunosorbent assays” or ELISAs for short. Cell-based ELISAs and capture ELISAs are very widely used. In preliminary work, the inventors checked whether either of these two methods is suitable for assay development. The cell-based ELISA, in which the cells are fixed in the microtiter plates and directly detected with phosphospecific antibodies, is basically suitable for detection. However, the detection only worked with a few antibodies and provided a very high background, so that further development did not seem to be promising. Experiments with a capture ELISA system, in which the cells are first lysed in the wells and receptors are then bound to capture antibodies on the bottom and sides of the wells, provided too low signal strength. Therefore, the inventors basically follow the solution approach of adapting cell lysis, receptor isolation and detection to the inventors' Western blot method as far as possible. After agonist treatment, the cells are lysed at 4° C. in a buffer containing detergents, which also contains phosphatase inhibitors and protease inhibitors. The lysates are then clarified by centrifugation. Receptor enrichment is then performed using antibody-coated agarose beads. Often, an additional epitope tag e.g. HA is inserted at the N-terminus of the receptor for this purpose and anti-HA agarose beads are used for receptor isolation. The beads are then washed by multiple centrifugations, aspiration of the buffer solution and addition of fresh buffer solution. The receptors are then detached using SDS sample buffer and applied to an SDS-PAGE gel and detected by Western blot using phosphosite-specific antibodies. This procedure most readily translates to a bead-based ELISA. Here, cell lysates are incubated directly in microtiter plates with magnetic HA beads. The washing step is performed by placing the plate on a special magnet. Detection is then performed by adding the phosphosite-specific GPCR antibodies, which in turn can be detected directly in the wells of the microtiter plates by peroxidase-labelled secondary antibodies after adding a soluble enzyme substrate solution, e.g. ABTS. Indeed, initial testing for prototypical receptors such as the μ-opioid receptor (MOP), ß2-adrenergic receptor (ß2), and C5a complement receptor 1 (C5a1) yielded very promising results, with several phosphosite-specific antibodies found for each of these receptors that provided signals in the range of absorbance (OD at 405 nm) above 1.2 after stimulation. Absorbance values in unstimulated control cells were in the range of <0.2. Control samples of stimulated and unstimulated cells that did not express the receptor (untransfected cells) were also <0.2. Controls by omitting the phosphosite-specific antibodies (primary antibodies) were regularly 0.05. Detection is successful even with very small amounts of lysate, so the assay can be readily performed in 96- and 384-well format in which cells can be grown and treated directly in the wells. Furthermore, division of the lysate of a well is possible so that both receptor phosphorylation and total receptor content can be determined in parallel for each individual sample, which is necessary for correct quantification.

### Example 2

This invention involves a method of a bead-based immunoassay that allows rapid and quantitative determination of GPCR/7TM phosphorylation in a microplate format and in a high-throughput manner using phosphosite-specific antibodies.

The assay consists of the following components:
1. Phosphosite-specific GPCR/7TM antibodies that recognize the phospho-serine and -threonine residues listed in Table 1.
2. GPCR/7TM antibodies that can recognize the receptors in a phosphorylation-independent manner.
3. GPCRs, which are epitope-tagged and can be expressed transiently or stably in cells in the form of plasmids or viral expression systems.
4. Magnetic beads (alternatively, agarose beads) loaded with antibodies that recognize the epitope tag of the receptor. Alternatively, lectin-coated beads (e.g. wheat germ lectin) or streptavidin beads are also possible.
5. Enzyme- or fluorescence-labeled secondary antibodies or a biotin-avidin-based detection system. The enzyme used is peroxidase or alkaline phosphatase.
6. Enzyme substrate (e.g. ABTS or TMB), for detection via optical density measurement or chemiluminescence.

In performing the assay, cells are first spread in 96- or 384-well F-bottom cell culture plates and cultured until confluency of greater than 90% is achieved. The cells are then treated with the agonists, antagonists, or inhibitors to be tested for sufficient time. The cells are then digested in a detergent-containing cell lysis buffer to which suitable phosphatase and protease inhibitors have also been added. Typically, cell lysis is performed for 30 min at 4° C. under agitation on a commercial microplate shaker. The lysate is then cleared. For this purpose, the lysates are centrifuged for 10 to 30 min at 4° C. The supernatant is then transferred to U-bottom assay plates and incubated with a sufficient amount of magnetic antibody beads. Typically, incubation is for 2 h at 4° C. with agitation on a microplate shaker. The microplate is then mounted on a suitable magnet and washed with PBS-Tween 20. The magnet thereby allows the supernatant to be decanted or aspirated while the beads are held at the bottom of the wells. Washing can be done either manually with a commercially available magnet or with an automatic microplate washer. Beads are then blocked with 1% BSA in PBS-Tween 20 and then incubated either under agitation for 2 hours at room temperature or overnight (12 to 16 hours) at 4° C. with phosphosite-specific GPCR/7TM antibodies in PBS-Tween 20 containing 1% BSA. After another wash, the beads are incubated with HRP-labeled secondary antibodies for 2 hours at room temperature. After a final wash, reaction with a dye substrate (e.g. ABTS) or chemiluminescence is performed. Finally, the optical density (at 405 nm for ABTS) is determined using a microplate reader. In particular, the assay is quantitative when the lysate of each sample is divided and one part is used to determine GPCR phosphorylation with a phosphosite-specific antibody and the other part is used to determine total receptor content with a phosphorylation-independent GPCR antibody.

### Example 3

### Protocol:

Seed approximately 100,000 cells per well in a poly-D- or poly-L-lysin-coated 96-F-well culture plate and grow to confluence of >95%.
Stimulate receptor with agonist for 5 to 30 min at 37° C.
Wash cells carefully with cold PBS (with Ca²⁺/Mg²⁺).
Lyse cells with 150 μl detergent-containing RIPA buffer plus inhibitor mix (1 tablet of phosphatase inhibitors and 1 tablet of protease inhibitors per 10 ml RIPA buffer).
Shake cells with RIPA inhibitor mix for 30 min for lysis at 4° C. and centrifuge for 20 min at 4000 rpm, 4° C.
Meanwhile prepare anti-HA antibody-coated magnetic beads.
   Use 2 μl bead solution/well (adjust as necessary depending on wash method etc.).
   Wash beads three times with RIPA.
   Resuspend beads in RIPA and dilute 1:20 in RIPA.
After centrifugation, transfer a total 100 μl of cleared lysate of each well into a 96-U-well plate. For parallel detection of phosphorylated and total receptor content, the lysate is divided into two parts. Transfer 80 μl lysate into one well (for detection of phosphorylated receptor content) and 20 μl lysate plus 60 μl RIPA into a parallel well of the corresponding 96-U-well plate. This procedure will result into two 96-U-well assay plates for one 96-F-well culture plate. For normalization of receptor phosphorylation to total receptor content, detection of both is be on the same assay plate.
Add 40 μl of previously prepared bead-RIPA suspension, resulting in a total volume of 100 μl in each well.
Agitate lysate-bead mixture for 2 h or overnight at 4° C.
Transfer plates onto a magnet and wash beads with PBS containing 0.1% Tween 20 three times with >100 μl/well in each well.
Blocking step: Add PBS with 0.1% Tween 20 and 1% BSA in a volume of 100 μl/well and agitate on a microplate shaker for 30 min at room temperature.
Decant blocking solution using the magnetic plate, add primary antibody at concentrations of 1 to 5 μg/ml in PBS with 0.1% Tween 20 and 1% BSA solution in a volume of 60 μl/well and agitate for 2 h to overnight at 4° C.
Wash three times with PBS+0.1% Tween 20.
Incubate with secondary antibody in 0.1% Tween 20 and 1% BSA in PBS solution (usually 1:200 to 1:1000) or overnight at room temperature.
Allow enzyme substrate (e.g. ABTS or derivative Super AquaBlue ELISA substrate or similar) to warm to room temperature.
Wash three times with PBS+0.1% Tween 20 and decant wash solution at the end.
Add 100 μl Super AquaBlue per well and allow to incubate for 2-20 min under agitation at room temperature.
Quench reaction with stop solution (for Super AquaBlue: add 100 μl/well 0.625 M oxalic acid)
Transfer assay plate onto a magnetic plate and transfer 140 μl the final reaction product into a new 96 F-well detection plate (avoid bubbles).
   Measure optical density (OD₄₀₅) using a microplate reader (at 405 nm for Super AquaBlue)
   Zero Readings should be below 0.2 and maximal readings should not exceed 1.2

All washing steps can also be performed automatically using a commercially available microplate washer.

### Example 4

Table 1 shows sequences as follows:

Underlined residues are phosphorylation sites. For immunization, cysteines within the sequence marked in bold were replaced by Abu (α-Aminobutyric acid). For all sequences, a cysteine was added to the N-terminus of the peptide (not shown) for immunization.

TABLE 1
Sequences

| | IUPHAR | | phosphorylated | |
|---|---|---|---|---|
| Receptor | Receptor | UniProt | sequence | Phosphorylatable |
| name | ID | ID | (SEQ ID NO.) | residue(s) |
| GPR35 | 102 | Q9HC97 | KAHKSQDSLCVTL | pS300/pS303 |
| | | | (SEQ ID NO. 23) | |
| | | | KSQDSLCVTLA | pS303/pT307 |
| | | | (SEQ ID NO. 24) | |
| GPR84 | 120 | Q9NQS5 | VSAATTQTLEG | pT263/pT264 |
| | | | (SEQ ID NO. 25) | |
| | | | LEGDSSEVGDD | pS271/pS272 |
| | | | (SEQ ID NO. 26) | |
| FFA4 | 127 | Q5NUL3 | GAILTDTSVK | pT347 |
| | | | (SEQ ID NO. 64) | |
| | | | ILTDTSVKRND | pT349/pS350 |
| | | | (SEQ ID NO. 65) | |
| | | | RNDLSIISG | PS357 |
| | | | (SEQ ID NO. 66) | |
| ACKR4 | 315 | Q9NPB9 | EFPFDSEGPTEPTST | pS338/pT342 |
| | | | (SEQ ID NO. 27) | |
| BB2 | 39 | P30550 | STGRSTTCMTS | pS354/pT355 |
| | | | (SEQ ID NO. 28) | |
| | | | TTCMTSLKSTN | pT359/pS360 |
| | | | (SEQ ID NO. 49) | |
| | | | TSLKSTNPSVA | pS363/pT364 |
| | | | (SEQ ID NO. 29) | |
| NTS1 | 309P-20 | P30989 | RKADSVSSNH | pS401 |
| | | | (SEQ ID NO. 30) | |
| | | | ADSVSSNHTLS | pS403/pS404 |
| | | | (SEQ ID NO. 31) | |
| PAC1 | 370 | P41586 | PSLASSGVNGG | pS437/pS438 |
| | | | (SEQ ID NO. 32) | |
| | | | VNGGTQLSILSKS | pS444/pS447 |
| | | | (SEQ ID NO. 52) | |
| | | | LSILSKSSSQIR | pS450/pS452 |
| | | | (SEQ ID NO. 33) | |
| UT | 365 | Q9UKP6 | GRSLSSCSPQP | pS360/pS361 |
| | | | (SEQ ID NO. 34) | |
| FPR3 | 224 | P25089 | DSAQTSNTDTT | pT334/pS335 |
| | | | (SEQ ID NO. 35) | |
| | | | QTSNTDTTSASP | pT337/pT339 |
| | | | (SEQ ID NO. 36) | |
| FPR2 | 223 | P25090 | PTNDTAANSA | pT335 |
| | | | (SEQ ID NO. 37) | |
| | | | PPAETELQAM | pT346 |
| | | | (SEQ ID NO. 38) | |
| APLNR | 36 | P35414 | SASYSSGHSQG | pS347/pS348 |
| | | | (SEQ ID NO. 39) | |
| NK1 | 360 | P25103 | LEMKSTRYLQT | pS338/pT339 |
| | | | (SEQ ID NO. 40) | |
| | | | SRLETTISTVV | pT356/pT357 |
| | | | (SEQ ID NO. 41) | |
| B2 | 42 | P30411 | QMENSMGTLRTSI | pS366/pT369 |
| | | | (SEQ ID NO. 67) | |
| | | | GTLRTSISVER | pT372/pS373 |
| | | | (SEQ ID NO. 53) | |
| PAR2 | 348 | P55085 | QVSLTSKKHSR | pT375/pS376 |
| | | | (SEQ ID NO. 42) | |
| | | | HSRKSSSYSSS | pS383/pS384 |
| | | | (SEQ ID NO. 43) | |
| | | | SSSYSSSSTTV | pS387/pS388 |
| | | | (SEQ ID NO. 44) | |
| CB2 | 57 | P34972 | RGLGSEAKEE | pS326 |
| | | | (SEQ ID NO. 45) | |
| | | | EAPRSSVTETE | pS335/pS336 |
| | | | (SEQ ID NO. 68) | |
| | | | RSSVTETEADGK | pT338/pT340 |
| | | | (SEQ ID NO. 54) | |
| NPS | 302 | Q6W5P4 | REQRSQDSRMTFRERT | pS346/pS349/TS352 |
| | | | (SEQ ID NO. 46) | |
| NPFF1 | 300 | Q9GZQ6 | WVRPSDSGLPSE | pS379/pS381 |
| | | | (SEQ ID NO. 47) | |
| | | | KEAYSERPGG | pS361 |
| | | | (SEQ ID NO. 50) | |
| | | | SGLPSESGPSSG | pS385/pS387 |
| | | | (SEQ ID NO. 48) | |
| CB1 | 57 | P21554 | PLDNSMGDSD | pS425 |
| | | | (SEQ ID NO. 55) | |
| | | | SMGDSDCLHK | pS429 |
| | | | (SEQ ID NO. 56) | |
| | | | NNAASVHRAA | pS441 |
| | | | (SEQ ID NO. 1) | |
| FFA2 | 226 | O15552 | RNQGSSLLGRR | pS296/pS297 |
| | | | (SEQ ID NO. 2) | |
| | | | RGKDTAEGTNEDRG | pT306/pT310 |
| | | | (SEQ ID NO. 3) | |
| FFA3 | 227 | O14843 | WQQESSMELKE | pS305/pS306 |
| | | | (SEQ ID NO. 4) | |
| | | | AERKTSEHSQG | pT328/pS329 |
| | | | (SEQ ID NO. 5) | |
| FPR1 | 222 | P21462 | DSTQTSDTATN | pT331/pS332 |
| | | | (SEQ ID NO. 57) | |
| | | | QTSDTATNSTLP | pT334/pT336 |
| | | | (SEQ ID NO. 58) | |
| | | | TATNSTLPSAE | pS338/pT339 |
| | | | (SEQ ID NO. 59) | |
| PAR1 | 222 | P25116 | DSTQTSDTATN | pT331/pS332 |
| | | | (SEQ ID NO. 60) | |
| | | | MDTCSSNLNNS | pS412/pS413 |
| | | | (SEQ ID NO. 51) | |
| | | | NLNNSIYKKL | pS418 |
| | | | (SEQ ID NO. 69) | |
| | | | QTSDTATNSTLP | pT334/pT336 |
| | | | (SEQ ID NO. 61) | |
| | | | TATNSTLPSAE | pS338/pT339 |
| | | | (SEQ ID NO. 62) | |
| NK3 | 362 | P29371 | LELKTTRFHPN | pT389/pT390 |
| | | | (SEQ ID NO. 6) | |
| | | | PNRQSSMYTVT | pS398/pS399 |
| | | | (SEQ ID NO. 7) | |
| | | | SSMYTVTRMESM | pT402/pT404 |
| | | | (SEQ ID NO. 8) | |
| GHSR | 246 | Q92847 | LKDESSRAWTE | pS355/pS356 |
| | | | (SEQ ID NO. 9) | |
| | | | SRAWTESSIN | pT360 |
| | | | (SEQ ID NO. 10) | |
| | | | AWTESSINT | pS362/pS363 |
| | | | (SEQ ID NO. 11) | |
| IP | 345 | P43119 | AHGDSQTPLSQL | pS319/pT321 |
| | | | (SEQ ID NO. 12) | |
| | | | QTPLSQLASGRRDP | pS324/pS328 |
| | | | (SEQ ID NO. 13) | |
| TP | 346 | P21731 | SLQPQLTQRSGLQ | pT337/pS340 |
| | | | (SEQ ID NO. 14) | |
| EP2 | 341 | P43116 | TQDATQTSCSTQ | pT342/pT344 |
| | | | (SEQ ID NO. 15) | |
| | | | DATQTSCSTQS | pT344/pS345 |
| | | | (SEQ ID NO. 16) | |
| | | | QTSCSTQSDAS | pS347/pT348 |
| | | | (SEQ ID NO. 17) | |
| EP4 | 343 | P35408 | GQHCSDSQRTSS | pS364/pS366 |
| | | | (SEQ ID NO. 18) | |
| | | | DSQRTSSAMSG | pT369/pS370 |
| | | | (SEQ ID NO. 19) | |
| | | | SSAMSGHSRSFIS | pS374/pS377 |
| | | | (SEQ ID NO. 63) | |
| GPR17 | 88 | Q13304 | GPPPSFEGKTNESSL | pS351/pT356 |
| | | | (SEQ ID NO. 20) | |
| | | | KTNESSLSAKSEL | pS359/pS360 |
| | | | (SEQ ID NO. 21) | |
| | | | NESSLSAKSEL | pS362/pS365 |
| | | | (SEQ ID NO. 22) | |
| 5HT4 | 9 | Q13639 | TINGSTHVLRD | pS354/pT355 |
| | | | (SEQ ID NO. 70) | |
| | | | ILGQIVPCSTVLRD | pT343/pS347/pT348 |
| | | | (SEQ ID NO. 71) | |
| ADB2 | 29 | P07550 | GNGYSSNGNTG | pS355/pT356 |
| | | | (SEQ ID NO. 72) | |
| | | | GNGYSSNGNTG | pT356 |
| | | | (SEQ ID NO. 73) | |
| | | | GNGYSSNGNTG | pS355 |
| | | | (SEQ ID NO. 74) | |
| | | | SNGNTGEQSGYHVE | pT360/pS364 |
| | | | (SEQ ID NO. 75) | |
| | | | SNGNTGEQSG | pT360 |
| | | | (SEQ ID NO. 76) | |
| | | | TGEQSGYHVE | pS364 |
| | | | (SEQ ID NO. 77) | |
| ACKR2 | 314 | O00590 | SLSSCSESSILTA | pS348/pS350 |
| | | | (SEQ ID NO. 78) | |
| ACKR3/CXCR7 | 314 | P25106 | ASRVSETEYSAL | pS350/pS352 |
| | | | (SEQ ID NO. 79) | |
| CCR5 | 62 | P51681 | PERASSVYTRS | pS336/pS337 |
| | | | (SEQ ID NO. 80) | |
| | | | SSVYTRSTGE | pT340 |
| | | | (SEQ ID NO. 81) | |
| | | | VYTRSTGEQE | pS342 |
| | | | (SEQ ID NO. 82) | |
| CCR10 | 67 | P46092 | APTETHSLSW | pT355 |
| | | | (SEQ ID NO. 83) | |
| | | | CSAPTETHSL | pT353 |
| | | | (SEQ ID NO. 84) | |
| CXCR4 | 71 | P61073 | VSRGSSLKILS | pS324/pS325 |
| | | | (SEQ ID NO. 85) | |
| | | | RGGHSSVSTES | pS337/pS338 |
| | | | (SEQ ID NO. 86) | |
| | | | TESESSSFHSS | pS346/pS347 |
| | | | (SEQ ID NO. 87) | |
| CXCR6 | 73 | O00574 | HQWKSSEDNSK | pS320/pS321 |
| | | | (SEQ ID NO. 88) | |
| | | | SEDNSKTFSASH | pS325/pT327 |
| | | | (SEQ ID NO. 89) | |
| | | | SKTFSASHNVEA | pS329/pS331 |
| | | | (SEQ ID NO. 90) | |
| CCK1 | 76 | P32238 | EEGGTTGASLS | pT408/pT409 |
| | | | (SEQ ID NO. 91) | |
| | | | TTGASLSRFSYS | pS412/pS414 |
| | | | (SEQ ID NO. 92) | |
| | | | LSRFSYSHMSAS | pS417/pS419 |
| | | | (SEQ ID NO. 93) | |
| CCK2 | 77 | P32239 | EDPPTPSIASL | pT427/pS429 |
| | | | (SEQ ID NO. 94) | |
| | | | LSRLSYTTISTL | pS437/pT439 |
| | | | (SEQ ID NO. 95) | |
| C5AR1 | 32 | P21730 | RNVLTEESVVRES | pT324/pS327 |
| | | | (SEQ ID NO. 96) | |
| | | | WVRESKSFTRST | pS332/pS334 |
| | | | (SEQ ID NO. 97) | |
| | | | SKSFTRSTVD | pT336 |
| | | | (SEQ ID NO. 98) | |
| | | | SFTRSTVDTMA | pS338/pT339 |
| | | | (SEQ ID NO. 99) | |
| | | | STVDTMAQKT | pT342 |
| | | | (SEQ ID NO. 100) | |
| C5AR2 | 33 | Q9P296 | DSKKSTSHDLVS | pS326/pT327 |
| | | | (SEQ ID NO. 101) | |
| CRFR1 | 212 | P34998 | MSIPTSPTRVS | pT428/pS429 |
| | | | (SEQ ID NO. 102) | |
| | | | PTSPTRVSFH | pT431 |
| | | | (SEQ ID NO. 103) | |
| | | | PTRVSFHSIKQST | pS434/pS437 |
| | | | (SEQ ID NO. 104) | |
| DRD1 | 214 | P21728 | LCPATNNAIE | pT354 |
| | | | (SEQ ID NO. 105) | |
| | | | AAMFSSHHEPR | pS372/pS373 |
| | | | (SEQ ID NO. 106) | |
| DRD2 | 215 | P14416 | HGLHSTPDSPA | pS317/pT318 |
| | | | (SEQ ID NO. 107) | |
| DRD5 | 218 | P21918 | SRTPVETVNISN | pT382/pT38 |
| | | | (SEQ ID NO. 108) | |
| GAL1 | 243 | P47211 | SRIDTPPSTN | pT340 |
| | | | (SEQ ID NO. 109) | |
| P2Y4 | 325 | P51582 | LALVSLPEDS | pS339 |
| | | | (SEQ ID NO. 110) | |
| | | | LPEDSSCRWAA | pS344/pS345 |
| | | | (SEQ ID NO. 111) | |
| | | | RWAATPQDSS | pT351 |
| | | | (SEQ ID NO. 112) | |
| P2Y12 | 328 | Q9H244 | ATSLSQDNRK | pS323 |
| | | | (SEQ ID NO. 113) | |
| NK1 | 360 | P25103 | RYLQTQGSVYKVS | pT344/pS347 |
| | | | (SEQ ID NO. 114) | |
| VPAC1 | 371 | P32241 | YRHPSGGSNGATC | pS422/pS425 |
| | | | (SEQ ID NO. 115) | |
| | | | SNGATCSTQVSML | pT429/pS431/pT432 |
| | | | (SEQ ID NO. 116) | |
| | | | STQVSMLTRVSPG | pS435/pT438 |
| | | | (SEQ ID NO. 117) | |
| VPAC2 | 372 | P41587 | RVCGSSFSRNG | pS408/pS409 |
| | | | (SEQ ID NO. 118) | |
| | | | GSSFSRNGSEGALQ | pS411/pS415 |
| | | | (SEQ ID NO. 119) | |
| | | | FHRGSRAQSFLQTE | pS425/pS429 |
| | | | (SEQ ID NO. 120) | |
| MOP | 319 | P35372 | IRQNTRDHPS | pT370 |
| | | | (SEQ ID NO. 121) | |
| | | | RDHPSTANTV | pS375 |
| | | | (SEQ ID NO. 122) | |
| | | | DHPSTANTVD | pT376 |
| | | | (SEQ ID NO. 123) | |
| | | | STANTVDRTN | pT379 |
| | | | (SEQ ID NO. 124) | |
| DOP | 317 | P41143 | VTACTPSDGP | pT361 |
| | | | (SEQ ID NO. 125) | |
| | | | ACTPSDGPGG | pS363 |
| | | | (SEQ ID NO. 126) | |
| KOP | 318 | P41145 | ERQSTSRVRNT | pS356/pT357 |
| | | | (SEQ ID NO. 127) | |
| | | | RVRNTVQDPA | pT363 |
| | | | (SEQ ID NO. 128) | |
| NOP | 320 | P41146 | DVQVSDRVRS | pS346 |
| | | | (SEQ ID NO. 129) | |
| | | | DRVRSIAKDV | pS351 |
| | | | (SEQ ID NO. 130) | |
| | | | LACKTSETVPR | pT362/pS363 |
| | | | (SEQ ID NO. 131) | |
| SST2 | 356 | P30874 | DGERSDSKQDKS | pS341/pS343 |
| | | | (SEQ ID NO. 132) | |
| | | | RLNETTETQRT | pT353/pT354 |
| | | | (SEQ ID NO. 133) | |
| | | | ETTETQRTLLNGD | pT356/pT359 |
| | | | (SEQ ID NO. 134) | |
| SST3 | 357 | P32745 | RRVRSQEPTVGPPE | pS337/pT341 |
| | | | (SEQ ID NO. 135) | |
| | | | PPEKTEEEDE | pT348 |
| | | | (SEQ ID NO. 136) | |
| | | | DGEESREGGK | PS361 |
| | | | (SEQ ID NO. 137) | |
| SST5 | 359 | P35346 | DADATEPRPD | pT333 |
| | | | (SEQ ID NO. 138) | |
| CB1 | 56 | P21554 | PLDNSMGDSD | pS425 |
| | | | (SEQ ID NO. 142) | |
| | | | SMGDSDCLHK | pS429 |
| | | | (SEQ ID NO. 144) | |
| | | | NNAASVHRAA | pS441 |
| | | | (SEQ ID NO. 140) | |
| ADB1 (β1) | 28 | P08588 | RRHATHGDRP | pT404 |
| | | | (SEQ ID NO. 141) | |
| | | | RPRASGCLAR | pS412 |
| | | | (SEQ ID NO. 143) | |
| A2A | 19 | P29274 | AAHGSDGEQV | pS329 |
| | | | (SEQ ID NO. 139) | |
| EP2 | 341 | P43116 | DATQTSCSTQS | pT344/pS345 |
| | | | (SEQ ID NO. 145) | |
| ADGRE2 | 184 | | RKLKTESEMHTL | |
| | | | (SEQ ID NO. 146) | |
| GHSR | 246 | | LKDESSRAWTE | pS355/pS356 |
| | | | (SEQ ID NO. 147) | |
| NPY1 | 305P | | DVSKTSLKQAS | |
| | | | (SEQ ID NO. 148) | |
| TP | 346 | P21731 | SLQPQLTQRSGLQ | pT337/pS340 |
| | | | (SEQ ID NO. 149) | |
| AGRB3 | 176 | | INADSSSSFPN | |
| | | | (SEQ ID NO. 150) | |
| EP2 | 341 | P43116 | TQDATQTSCSTQ | pT342/pT344 |
| | | | (SEQ ID NO. 151) | |
| EP3 | 342 | P43115 | NNYASSSTSLP | pS369/pS370 |
| | | | (SEQ ID NO. 152) | |
| GHSR | 246 | | AWTESSINT | pS362/pS363 |
| | | | (SEQ ID NO. 153) | |
| IP | 345 | P43119 | AHGDSQTPLSQL | pS319/pT321 |
| | | | (SEQ ID NO. 154) | |
| ADGRL3 | 208 | | KTSGSRTPGRYS | |
| | | | (SEQ ID NO. 155) | |
| CMKL2 | 82 | | SCSGTVSEQLRN | |
| | | | (SEQ ID NO. 156) | |
| hCX3CR1 | 74 | | DFSSSESQRSRH | |
| | | | (SEQ ID NO. 157) | |
| ADGRE5 | 177 | | GSKYSEFTSTTSG | |
| | | | (SEQ ID NO. 158) | |
| ADGRL3 | 208 | | GSGKTSGSRTP | |
| | | | (SEQ ID NO. 159) | |
| EP2 | 341 | P43116 | QTSCSTQSDAS | pS347/pT348 |
| | | | (SEQ ID NO. 160) | |
| EP3 | 342 | P43115 | YASSSTSLPCQ | pT372/pS373 |
| | | | (SEQ ID NO. 161) | |
| NPY1 | 305P | | TIAMSTMHTDV | |
| | | | (SEQ ID NO. 162) | |
| hCX3CR1 | 74 | | GSVLSSNFTYH | |
| | | | (SEQ ID NO. 163) | |
| | | | SRAWTESSIN | |
| | | | (SEQ ID NO. 164) | |
| CMKL2 | 82 | | LWEVSCSGTVSE | |
| | | | (SEQ ID NO. 165) | |
| AGRB3 | 176 | | AQIMTDFEKD | |
| | | | (SEQ ID NO. 166) | |
| EP3 | 342 | P43115 | PCQCSSTLMWS | pS379/pS380 |
| | | | (SEQ ID NO. 167) | |
| IP | 345 | P43119 | QTPLSQLASGRRDP | pS324/pS328 |
| | | | (SEQ ID NO. 168) | |
| CMKL2 | 82 | | QLRNSETKNLCL | |
| | | | (SEQ ID NO. 169) | |
| ADGRE2 | 184 | | AKADTSKPSTV | |
| | | | (SEQ ID NO. 170) | |
| DP2 | 339 | Q9Y5Y4 | GGAGSSRRRRT | pS338/pS339 |
| | | | (SEQ ID NO. 171) | |
| hCX3CR1 | 74 | | ESQRSRHGSVLSSN | |
| | | | (SEQ ID NO. 172) | |
| hCB1 | 56 | P21554 | NNAASVHRAA | pS441 |
| | | | (SEQ ID NO. 173) | |
| AGRB3 | 176 | | ADSSSSFPNGH | |
| | | | (SEQ ID NO. 174) | |
| NPY1 | 305P | | STMHTDVSKTSLK | |
| | | | (SEQ ID NO. 175) | |
| ADGRL3 | 208 | | SGKSTESSIGSG | |
| | | | (SEQ ID NO. 176) | |
| ADGRE2 | 184 | | MHTLSSSAKAD | |
| | | | (SEQ ID NO. 177) | |
| hADB1 (β1) | 28 | P08588 | RRHATHGDRP | pT404-ß1 |
| | | | (SEQ ID NO. 178) | |
| ADGRE5 | 177 | | FTSTTSGTGHN | |
| | | | (SEQ ID NO. 179) | |
| DP2 | 339 | Q9Y5Y4 | RRRRTSSTARS | pS344/pS345 |
| | | | (SEQ ID NO. 180) | |
| ADGRE5 | 177 | | TTSGTGHNQTRALRA | |
| | | | (SEQ ID NO. 181) | |
| hCXCR2 | 69 | | VGSSSGHTSTTL | |
| | | | (SEQ ID NO. 182) | |
| hCB1 | 56 | P21554 | SMGDSDCLHK | pS429 |
| | | | (SEQ ID NO. 183) | |
| hADB1 (β1) | 28 | P08588 | RPRASGCLAR | pS412 |
| | | | (SEQ ID NO. 184) | |
| hCB1 | 56 | P21554 | PLDNSMGDSD | pS425 |
| | | | (SEQ ID NO. 185) | |
| hCXCR2 | 69 | | SFVGSSSGHTS | |
| | | | (SEQ ID NO. 186) | |
| hCXCR2 | 69 | | DSRPSFVGSS | |
| | | | (SEQ ID NO. 187) | |
| 5HT6 | 11 | P50406 | ASLASPSLRTSH (SEQ ID | pS350/pS352-5HT6 |
| | | | NO: 188) | |
| 5HT6 | 11 | P50406 | PSLRTSHSGPR (SEQ ID | pT355/pS356-5HT6 |
| | | | NO: 189) | |
| M1 | 13 | P11229 | EEPGSEVVIK (SEQ ID | pS298-M1 |
| | | | NO: 190) | |
| M2 | 14 | P08172 | RDPVTENCVQ (SEQ ID | pT307/pS309-M2 |
| | | | NO: 191) | |
| M2 | 14 | P08172 | EEKESSNDSTS (SEQ ID | pT310/pS311-M2 |
| | | | NO: 192) | |
| M2 | 14 | P08172 | SSNDSTSVSAVA (SEQ ID | pS315/pS320-M2 |
| | | | NO: 193) | |
| M2 | 14 | P08172 | QDENTVSTSLGH (SEQ ID | pS323/pS324-M2 |
| | | | NO: 194) | |
| M2 | 14 | P08172 | NTVSTSLGHSK (SEQ ID | pS332/pS333/pS334-M2 |
| | | | NO: 195) | |
| M2 | 14 | P08172 | SLGHSKDENSKQTCI | pS351/pS352-M2 |
| | | | (SEQ ID NO: 196) | |
| M3 | 15 | P20309 | DQDHSSSDSWNN (SEQ | pS332/pS333/pS334-M3 |
| | | | ID NO: 197) | |
| M3 | 15 | P20309 | EDIGSETRAIYS (SEQ ID | pS359/pT361-M3 |
| | | | NO: 198) | |
| M4 | 16 | P08173 | TSNESSSGSAT (SEQ ID | pS296/pS297-M4 |
| | | | NO: 199) | |
| M4 | 16 | P08173 | ERPATELSTTEA (SEQ ID | pT311/pS314-M4 |
| | | | NO: 200) | |
| A2A | 19 | P29274 | AAHGSDGEQV (SEQ ID | pS329-A2A |
| | | | NO: 201) | |
| AT1 | 34 | P3056 | HSNLSTKMSTL (SEQ ID | pS331/pT332-AT1 |
| | | | NO: 202) | |
| AT1 | 34 | P3056 | STKMSTLSYRP (SEQ ID | pS335/pT336-AT1 |
| | | | NO: 203) | |
| AT1 | 34 | P3056 | SDNVSSSTKKP (SEQ ID | pS346/pS347-AT1 |
| | | | NO: 204) | |
| PTH1 | 331 | Q03431 | SGSSSYSYGPMV (SEQ | pS493/pS495-PTH1 |
| | | | ID NO: 205) | |
| PTH1 | 331 | Q03431 | MVSHTSVTNVG (SEQ ID | pT503/pS504-PTH1 |
| | | | NO: 206) | |
| TSHR | 255 | P16473 | PPKNSTDIQVQ (SEQ ID | pS716/pT717-TSHR |
| | | | NO: 207) | |
| V2 | 368 | P30518 | RTPPSLGPQD (SEQ ID | pS350-V2 |
| | | | NO: 208) | |
| V2 | 368 | P30518 | DESCTTASSSL (SEQ ID | pT359/pT360-V2 |
| | | | NO: 209) | |
| V2 | 368 | P30518 | TTASSSLAKDT (SEQ ID | pS362/pS363/pS364-V2 |
| | | | NO: 210) | |
| BB3/BRS-3 | 40 | P32247 | PVADTSLTTLA (SEQ ID | pT360/pS361-BB3 |
| | | | NO: 211) | |
| BB3/BRS-3 | 40 | P32247 | DTSLTTLAVMG (SEQ ID | pT363/pT364-BB3 |
| | | | NO: 212) | |
| BB3/BRS-3 | 40 | P32247 | AVMGTVPGTG (SEQ ID | pT370-BB3 |
| | | | NO: 213) | |
| NPY2 | 306 | P49146 | DAIHSEVSVTFKA (SEQ ID | pS351/pS354-NPY2 |
| | | | NO: 214) | |
| NPY2 | 306 | P49146 | HSEVSVTFKAKK (SEQ ID | pS354/pS356-NPY2 |
| | | | NO: 215) | |
| NPY2 | 306 | P49146 | VRKNSGPNDSFTEAT | pS359/pS364-NPY2 |
| | | | (SEQ ID NO: 216) | |
| NPY1 | 305 | P25929 | STMHTDVSKTSLK (SEQ | pT357/pS360-NPY1 |
| | | | ID NO: 217) | |
| NPY1 | 305 | P25929 | DVSKTSLKQAS (SEQ ID | pT362/pS363-NPY1 |
| | | | NO: 218) | |
| TRH | 363 | P34981 | VIKESDHFST (SEQ ID | pS360-TRH |
| | | | NO: 219) | |
| TRH | 363 | P34981 | SDHFSTELDDI (SEQ ID | pS364/pT365-TRH |
| | | | NO: 220) | |
| TRH | 363 | P34981 | LDDITVTDTY (SEQ ID | pST371-TRH |
| | | | NO: 221) | |
| M3 | 15 | P20309 | TKLPSSDNLQV (SEQ ID | pS385/pS386-M3 |
| | | | NO: 222) | |
| ETB | 220 | P2450 | DNFRSSNKYSS (SEQ ID | pS435/pS436-ETB |
| | | | NO: 223) | |
| CXCR2 | 69 | P25025 | VGSSSGHTSTTL (SEQ ID | pT347-CXCR2 |
| | | | NO: 224) | |
| CXCR2 | 69 | P25025 | SFVGSSSGHTS (SEQ ID | pS351/pS352-CXCR2 |
| | | | NO: 225) | |
| CXCR2 | 69 | P25025 | DSRPSFVGSS (SEQ ID | pS353/pT356-CXCR2 |
| | | | NO: 226) | |

### Example 5: Development of a 7TM Phosphorylation Assay

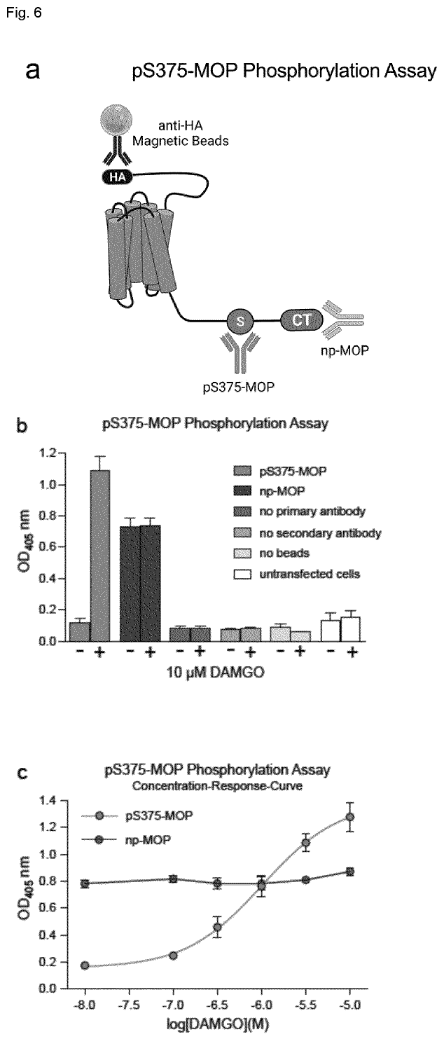

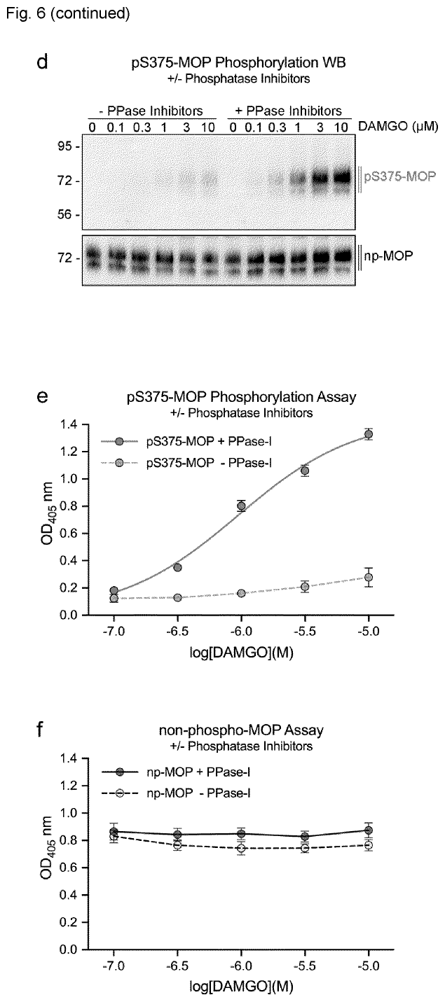

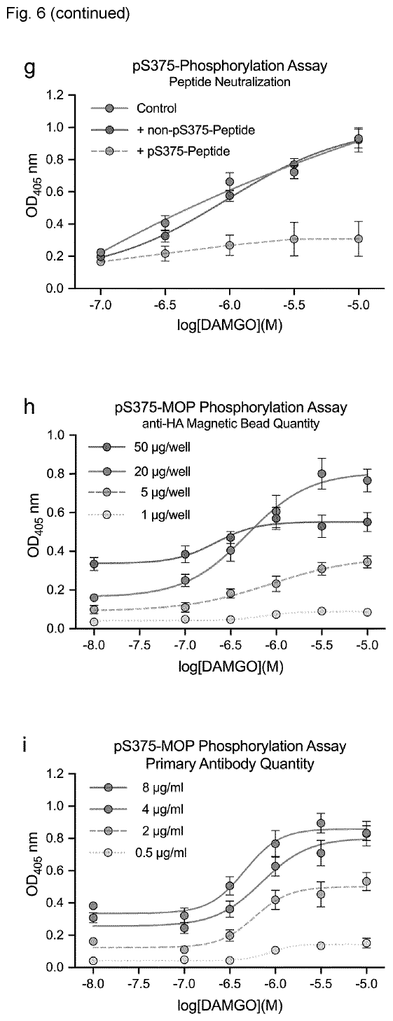

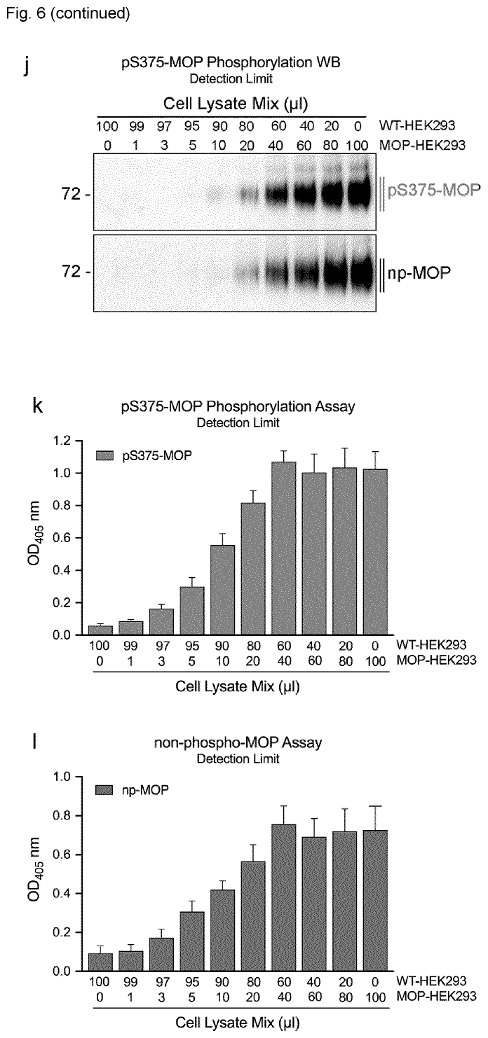

This study was performed to develop a quantitative GPCR phosphorylation immunoassay. To this end, HEK293 cells stably expressing hemagglutinin (HA)-tagged mouse μ-opioid receptors (MOPs) were seeded into poly-L-lysine-coated F-bottom 96-well plates and grown to >95% confluency. The cells were cultured in the presence or absence of the agonist [D-Ala2,N-MePhe4,Gly-ol]-enkephalin (DAMGO) and then lysed in detergent buffer containing protease and protein phosphatase (PPase) inhibitors. After plate centrifugation, lysates were transferred to U-bottom 96-well plates. MOPs were then immunoprecipitated using mouse anti-HA antibody-coated magnetic beads. After washing the beads under magnetic force, either phosphosite-specific rabbit antibodies that specifically recognized the S375-phosphorylated form of MOP (pS375-MOP) or antibodies that detected MOP in a phosphorylation-independent manner (np-MOP) were added to the cells (FIG. 6a). Primary antibody binding was then detected using commercially available enzyme-labeled secondary antibodies followed by addition of the respective enzyme substrate solution. The color reaction in DAMGO-treated wells was stopped when the optical density (OD) read at 405 nm reached 1.2, typically within 2 to 8 min. Under identical conditions, the OD of untreated wells was approximately 0.2, suggesting that DAMGO-induced S375 phosphorylation of MOP was specifically detected within the optimal dynamic range for 2′-azino-bis-(3-ethylbenzthiazoline-6-sulfonic acid (ABTS)-based enzyme immunoassay substrates (FIG. 6b). Omission of the magnetic beads or the primary or secondary antibodies or the use of untransfected cells resulted in OD readings of 0.2 or less. When DAMGO was added at different concentrations ranging from 10 nM to 10 UM in wells assayed using the anti-pS375-MOP antibody, the inventors observed increasing OD readings with data points following the typical shape of a sigmoidal concentration-response curve (FIG. 6c). In contrast, all wells assayed using the anti-np-MOP antibody yielded an OD reading of approximately 0.8, independent of agonist exposure, indicating that all the MOPs had been identified (FIG. 6c). Next, the inventors evaluated the effect of PPase inhibitors with both a pS375-MOP phosphorylation immunoassay and Western blot assay with cells cultured in a multiwell plate. For the Western blot analysis, cells were cultured and treated in 96-well plates as described and lysed in detergent buffer with or without PPase inhibitors. MOPs were immunoprecipitated with anti-HA magnetic beads. Immunoprecipitates were washed under magnetic force, and receptors were eluted from the beads into SDS-sample buffer by incubating the plates at 43° C. for 25 min. When these samples were immunoblotted with the anti-pS375-MOP antibody, a concentration-dependent increase in S375 phosphorylation was observed in samples cultured with PPase inhibitors but not in samples lysed without PPase inhibitors (FIG. 6d, top panel). When the blot was stripped and reprobed with the np-MOP antibody, similar levels of MOPs were detected in all the samples irrespective of the presence of PPase inhibitors or agonist exposure (FIG. 6d, bottom panel). For the in-well assay, cells were plated and grown, treated and lysed as in the Western blot assay, except that the samples were directly immunoassayed in the plate. Similar to the results obtained by immunoblotting, the results of the assay performed with the anti-pS375-MOP antibody revealed a concentration-dependent increase in signal intensity only in the wells with PPase inhibitor-treated samples (FIG. 6e). In contrast, in wells assayed with the anti-np-MOP antibody, similar signals were observed independent of inhibitor or agonist treatment (FIG. 6f). Notably, a high degree of correlation between the concentration-response curves obtained through the immunoblot analysis and immunoassay was found. These results show that the addition of PPase inhibitors during cell lysis was an essential component of the assay. These results also show that anti-pS375-MOP antibody binding depended on agonist-induced receptor phosphorylation, which needed to be protected from PPase digestion during cell lysis. The anti-np-MOP antibody bound to receptors regardless of their phosphorylation status, and hence, using this antibody appeared to be a feasible means of controlling total receptor content.

### Example 6: Validation of the 7TM Phosphorylation Assay

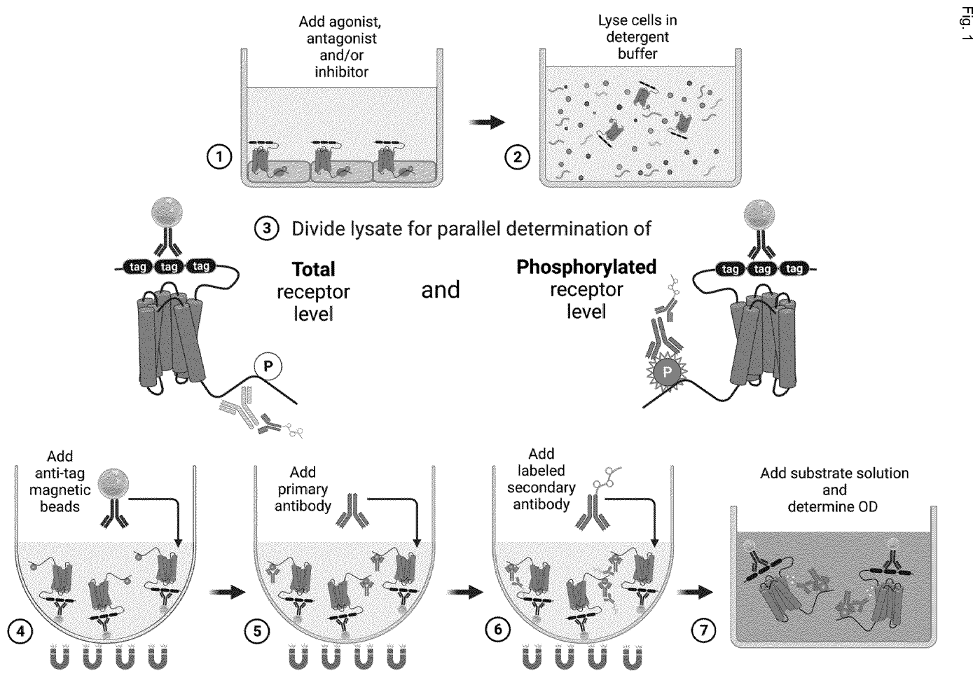

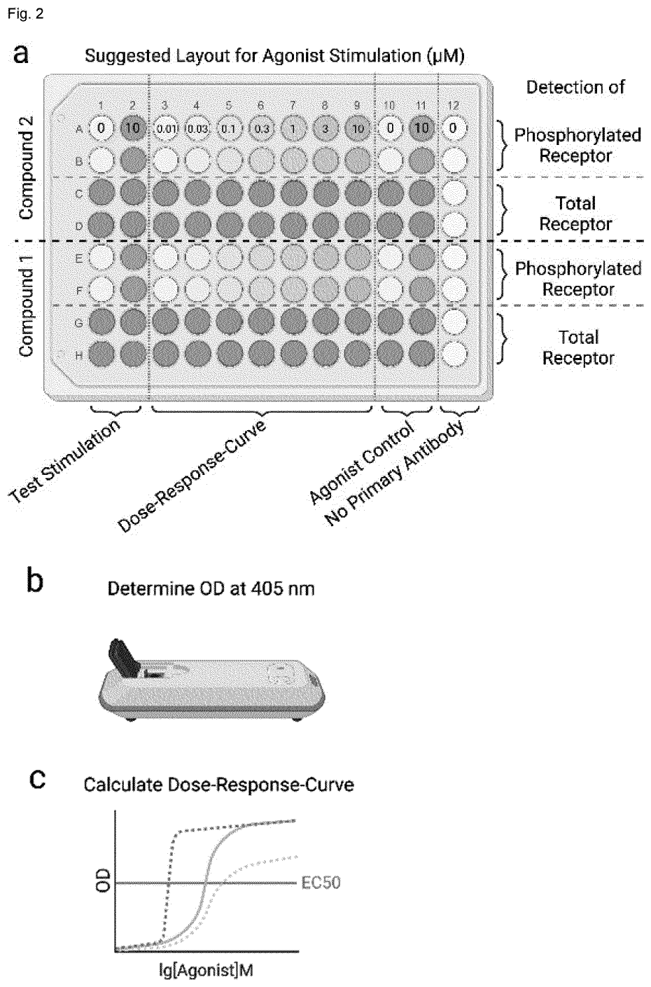

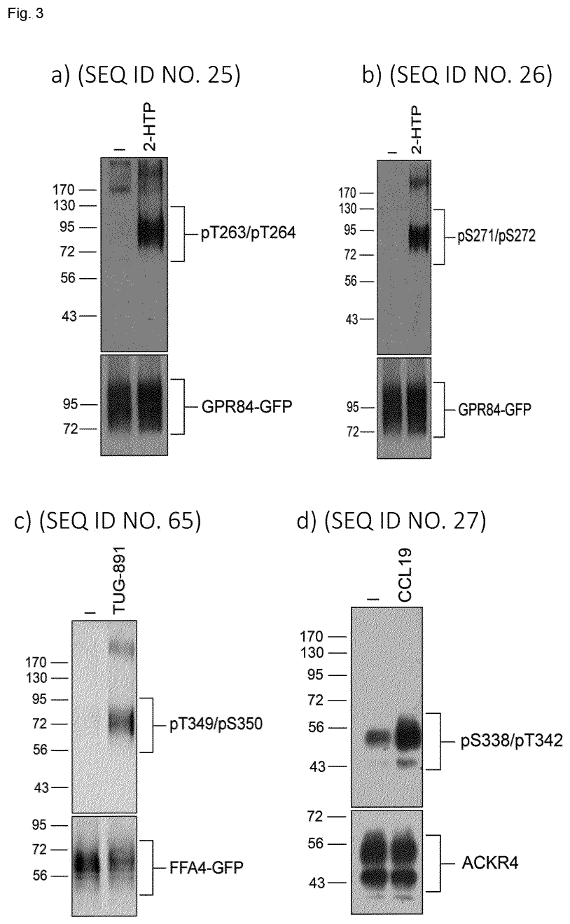

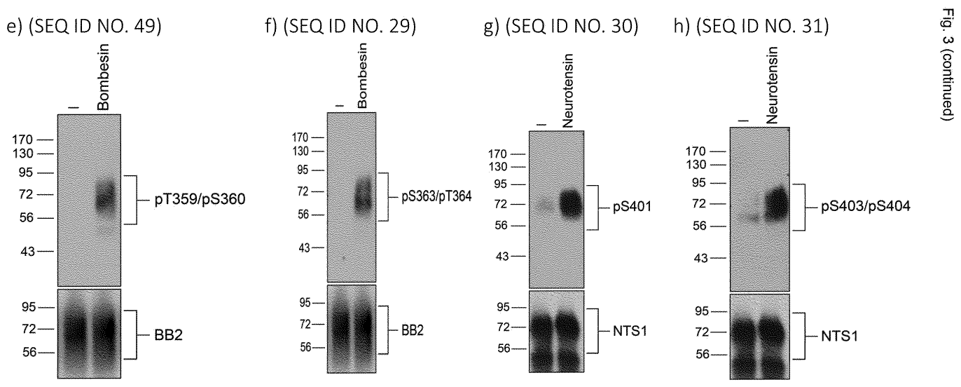

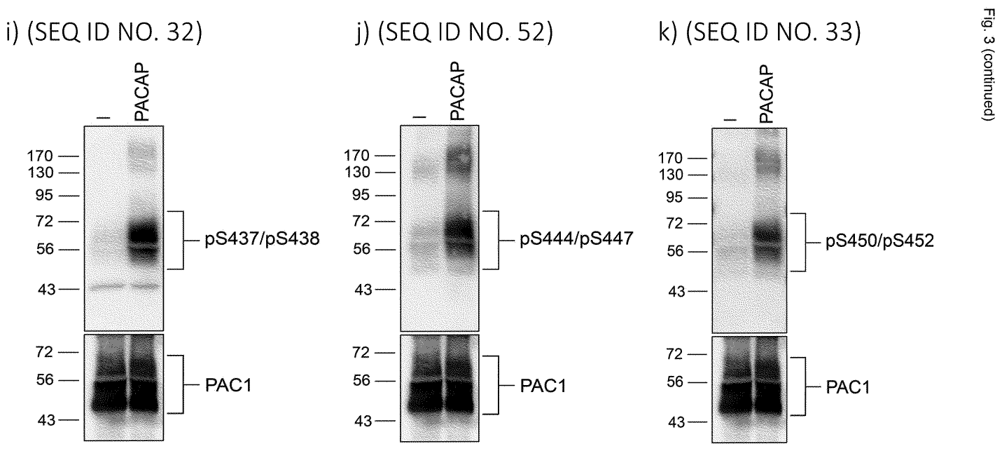

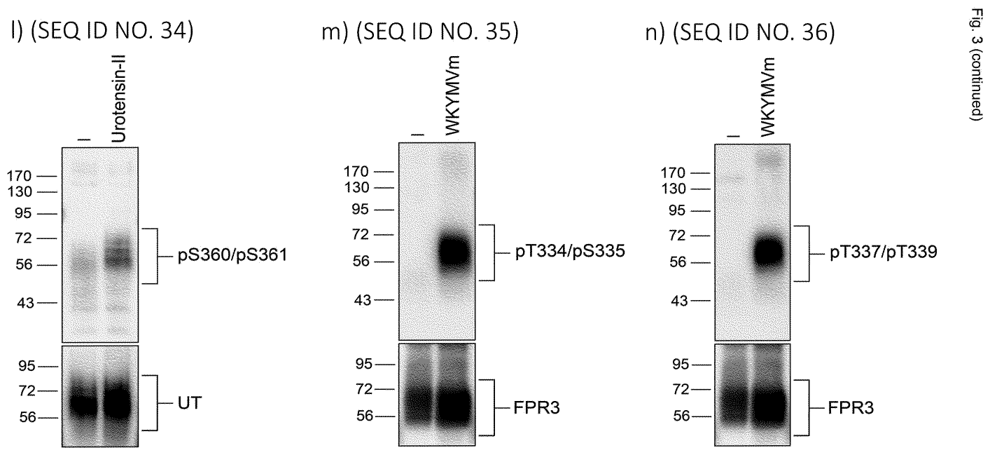

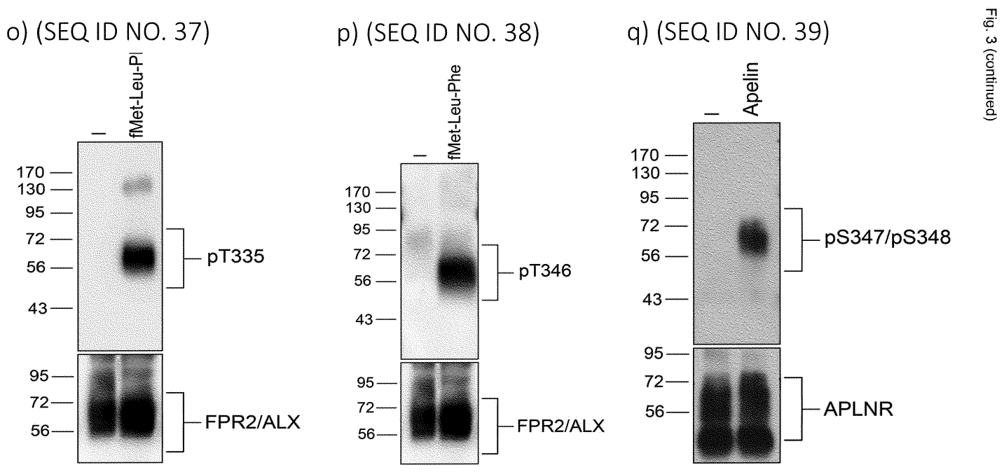

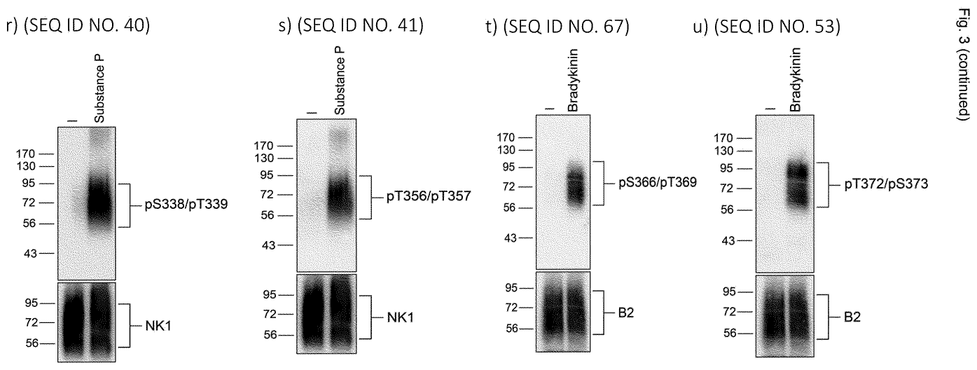

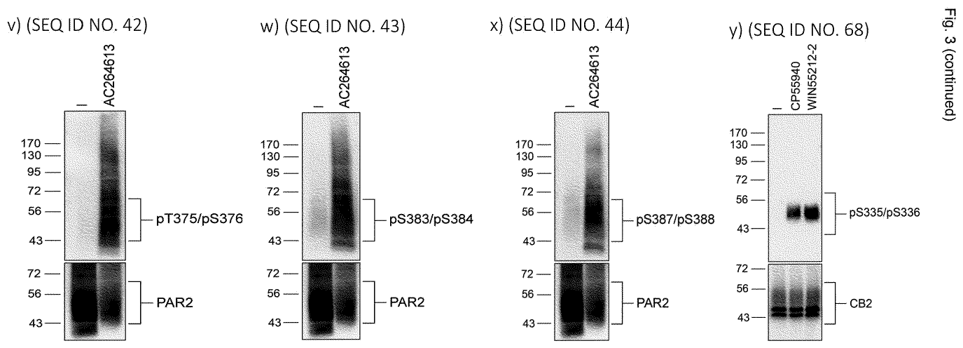

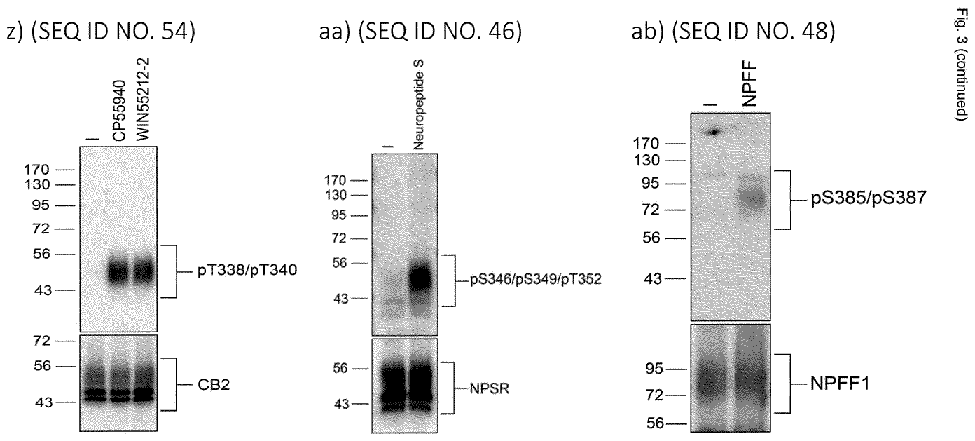

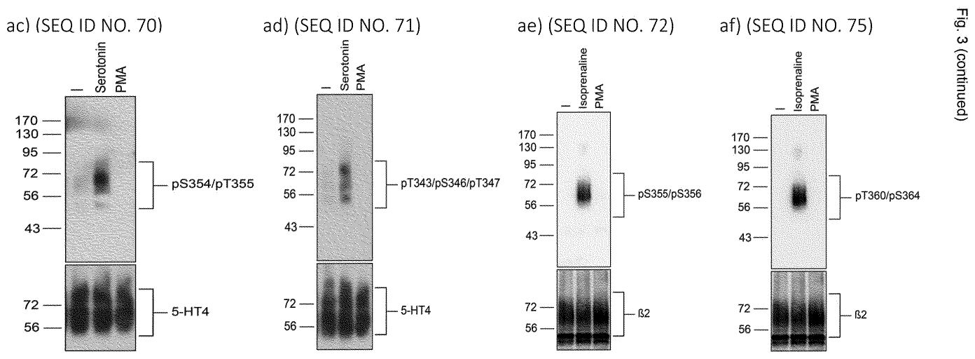

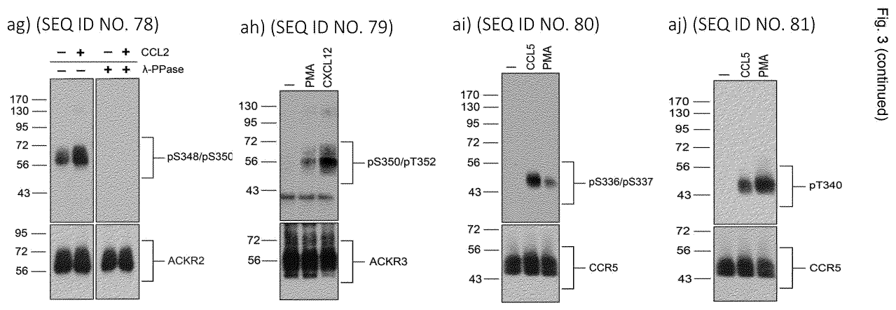

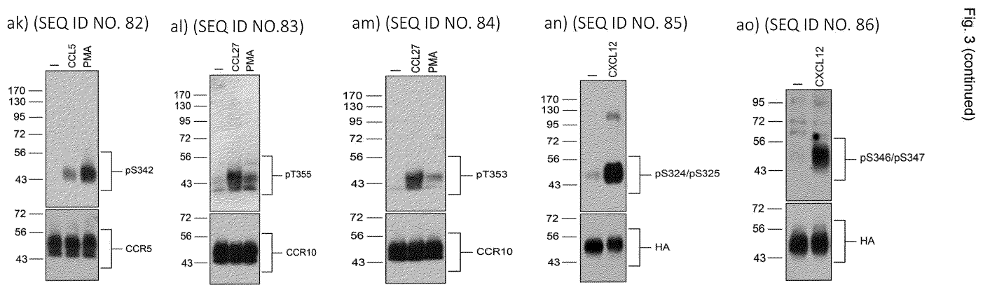

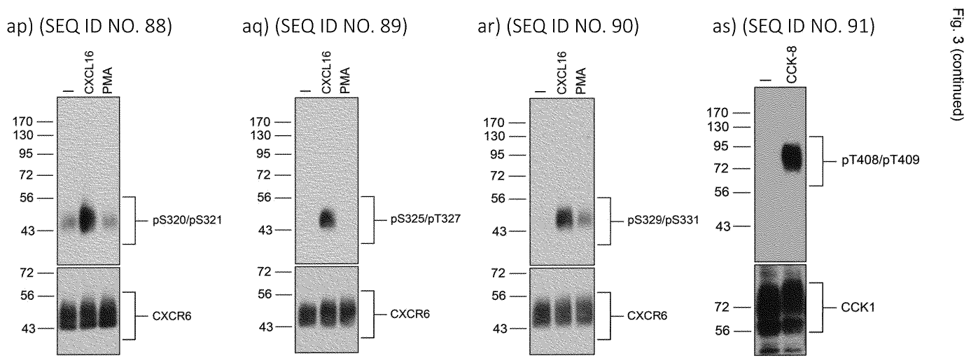

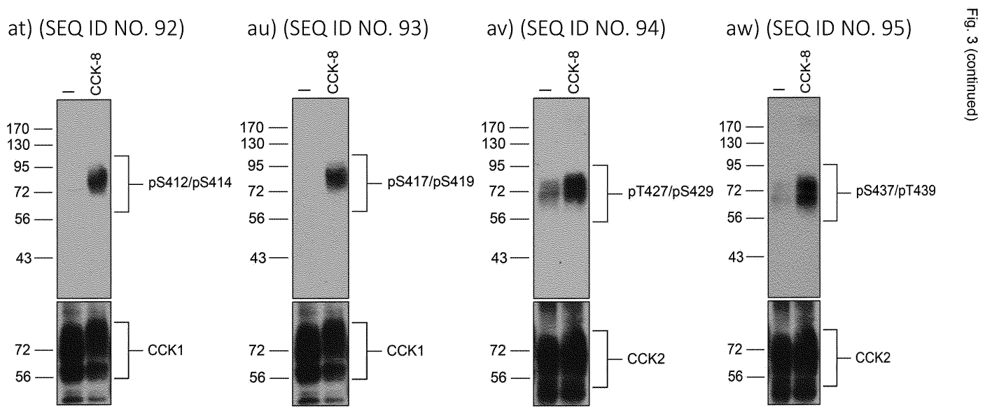

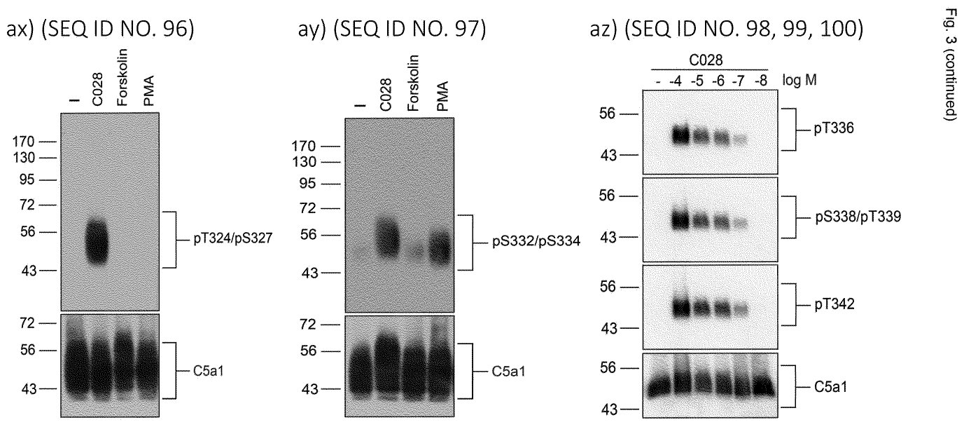

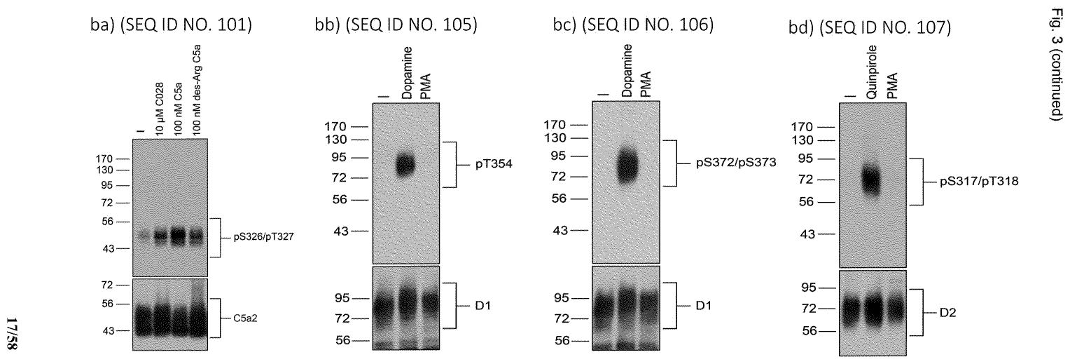

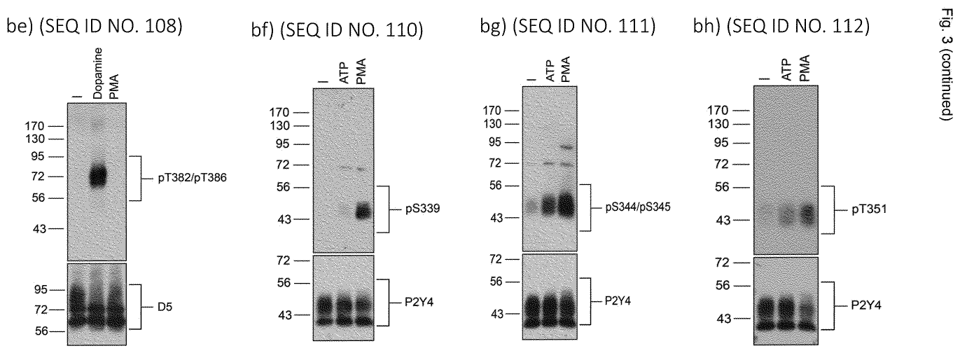

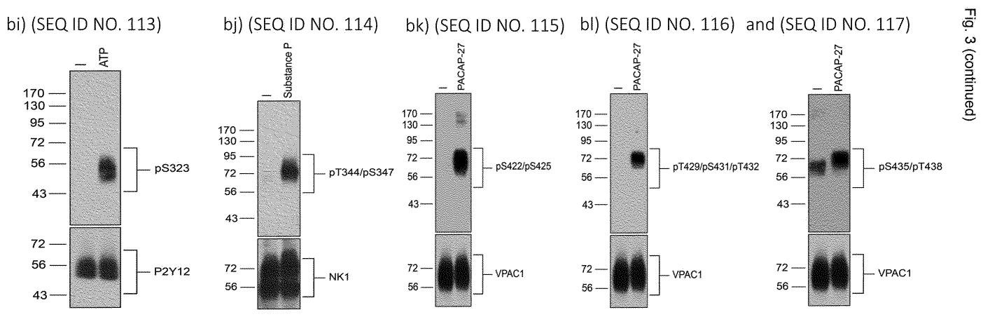

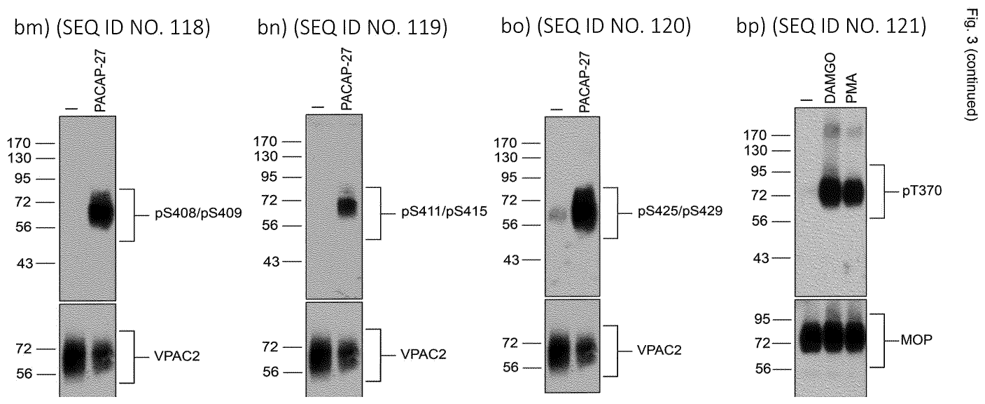

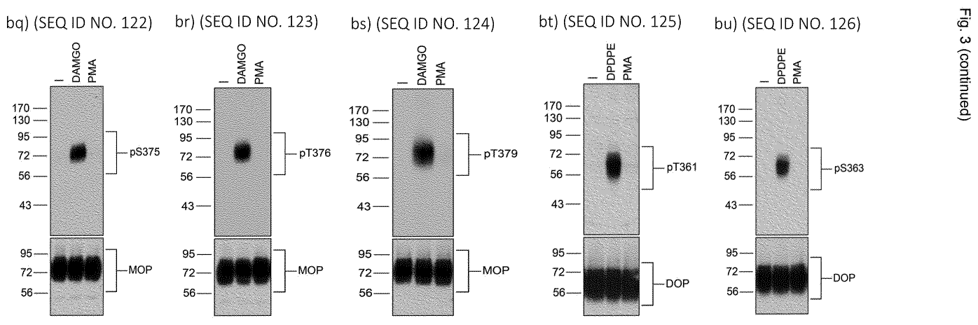

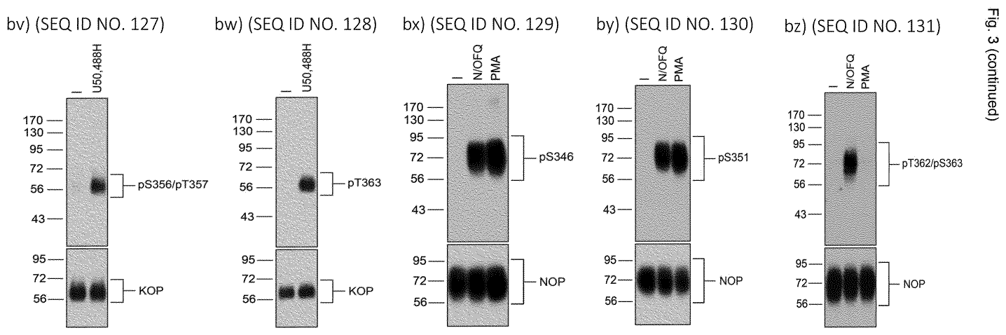

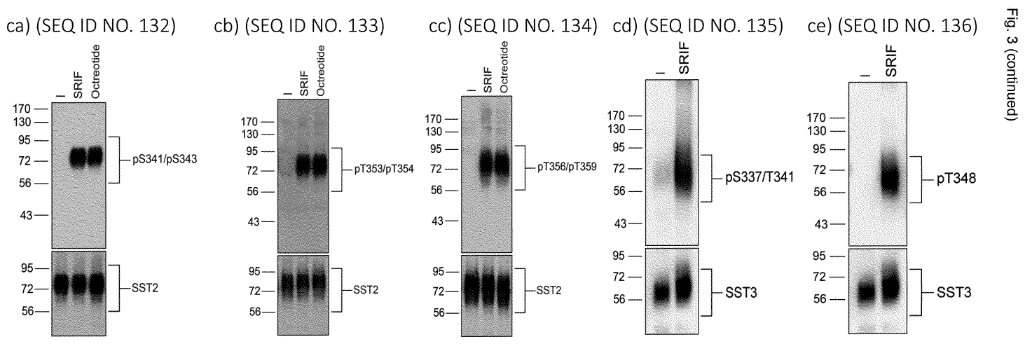

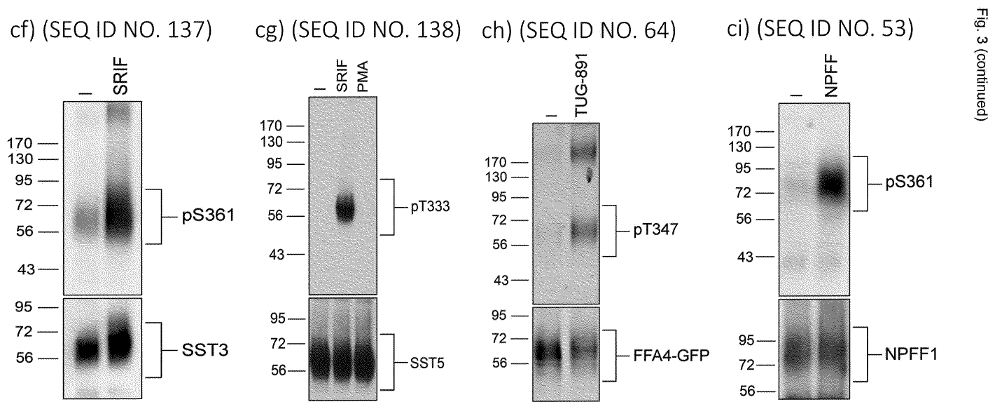

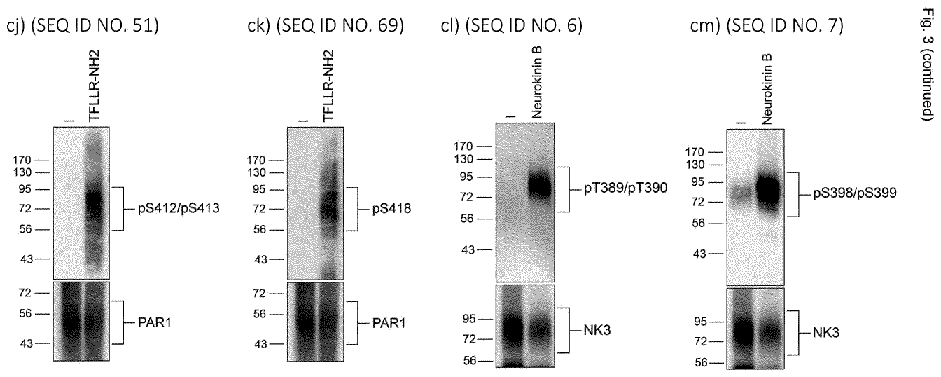

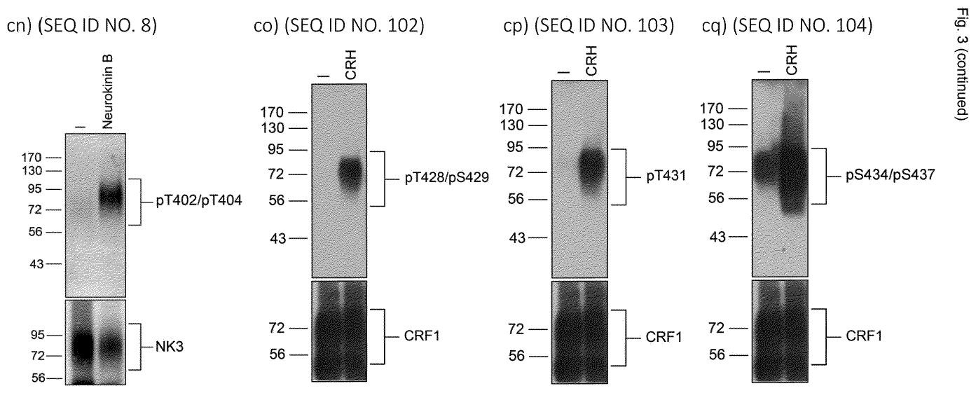

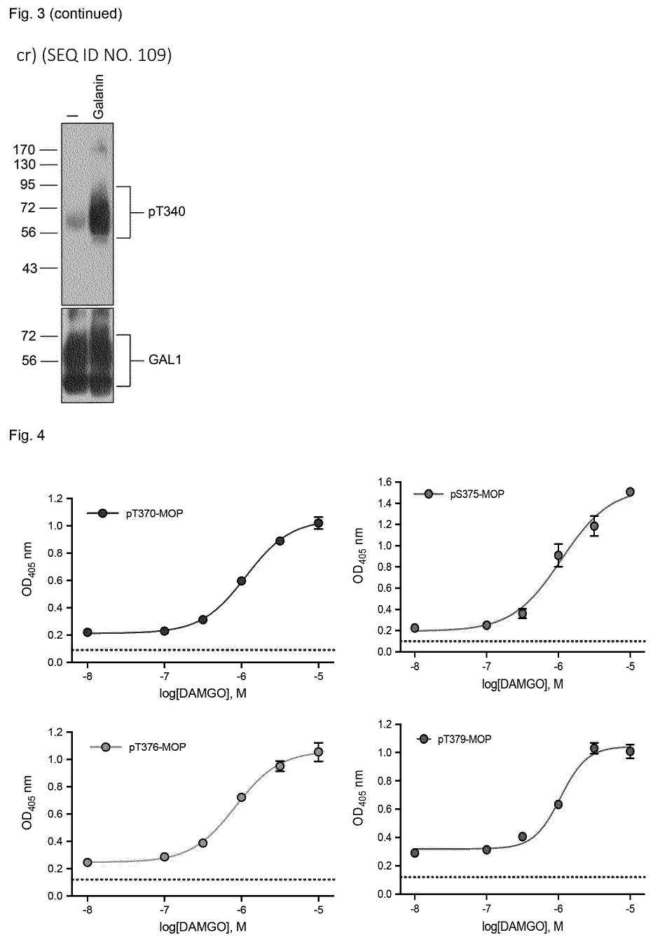

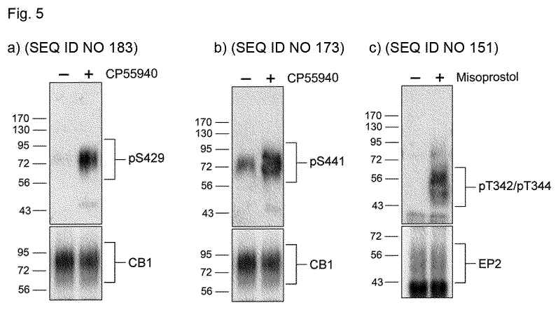

To substantiate the specific binding of the anti-pS375-MOP antibody, the inventors performed peptide neutralization to introduce controls in the immunoassay. When the anti-pS375-MOP antibody was incubated with an excessive amount immunizing peptide that contained phosphorylated S375, the concentration-dependent increase in signal intensity was completely blocked (FIG. 6g). In contrast, the immunoassay was unaffected by the addition of the corresponding unphosphorylated peptide, indicating unequivocal detection of agonist-dependent S375 MOP phosphorylation under these conditions (FIG. 6g). The inventors next optimized individual assay components such as the quantity of magnetic beads and primary antibody required. The magnetic beads used in this study had a diameter of 1 μm, and 1 μl of the slurry contained 10 μg of these beads. As shown, the addition of 20 μg of magnetic beads per well resulted in high signal intensity with limited background (FIG. 6h). Increasing to 50 μg of magnetic beads did not enhance the signal strength but resulted in a higher background. The signal intensity and the dynamic range of the assay decreased when using 5 μg beads or less (FIG. 6h). The inventors also found that the addition of 4 μg/ml of the anti-pS375-MOP antibody yielded results within the desired signal-to-noise ratio (FIG. 6i). Nevertheless, the optimal quantity of primary antibody needed to be individually determined for each phosphosite-specific antibody. Having established optimal assay conditions, the inventors sought to determine the detection limit. To this end, MOP-transfected cells (MOP-HEK293) and untransfected cells (WT-HEK293) were cultured in separate wells and exposed to saturating concentrations of DAMGO. The MOP-HEK293 cell lysate was stepwise diluted with WT-HEK293 lysate to yield a total volume of 100 μl in each sample, which represented a total of 70,000 cells (FIG. 6j). These treated samples were then evaluated via immunoblot analysis and anti-pS375-MOP phosphorylation immunoassay. In both assays, strong signals were detected in the samples containing at least 40 to 60 μl of the MOP-HEK293 lysate. Further dilution resulted in a linear decline in signal intensity (FIG. 6j-l). The detection limit of the Western blot assays was reached when samples contained less than 20 μl of the MOP-HEK293 lysate (FIG. 6j). In both the pS375-MOP and np-MOP immunoassays, the limit of detection was reached when samples contained less than 5 μl of the MOP-HEK293 lysate (FIG. 6l). Saturation-binding studies revealed that the MOP-HEK293 cells expressed 300,000 functional MOPs per cell, allowing us to calculate a detection limit of 80 pg receptor protein for the pS375-MOP immunoassay and 500 pg for the immunoblot assay. In the range of 80 μg to 1200 pg receptor protein, the pS375-MOP immunoassay showed a linear increase in signal intensity. In recovery experiments, the lysates were subjected to a second round of bead-based immunoprecipitation. The results show that the MOPs were not completely removed from the lysates containing more than 40 μl of the original MOP-HEK293 cell lysate. The observation that the pS375-MOP immunoassay yielded linear results using only 50% of the lysate was in each well led the inventors to divide each lysate for the detection of both phosphorylated and total receptor content, thus enabling a true quantitative analysis. Therefore, the step in which the lysate was divided was integrated into the standard workflow of all subsequent GPCR phosphorylation assays (FIG. 1). The agonist setting allows the assessment of concentration-response curves as a percentage of maximal stimulation by endogenous or synthetic agonists. Finally, the inventors used the 7TM phosphorylation assay to unequivocally detected agonist-induced receptor phosphorylation in brain lysates obtained from HA-MOP knock-in mice.

### Example 7: Ligand Profiling in MOP Phosphorylation Assays

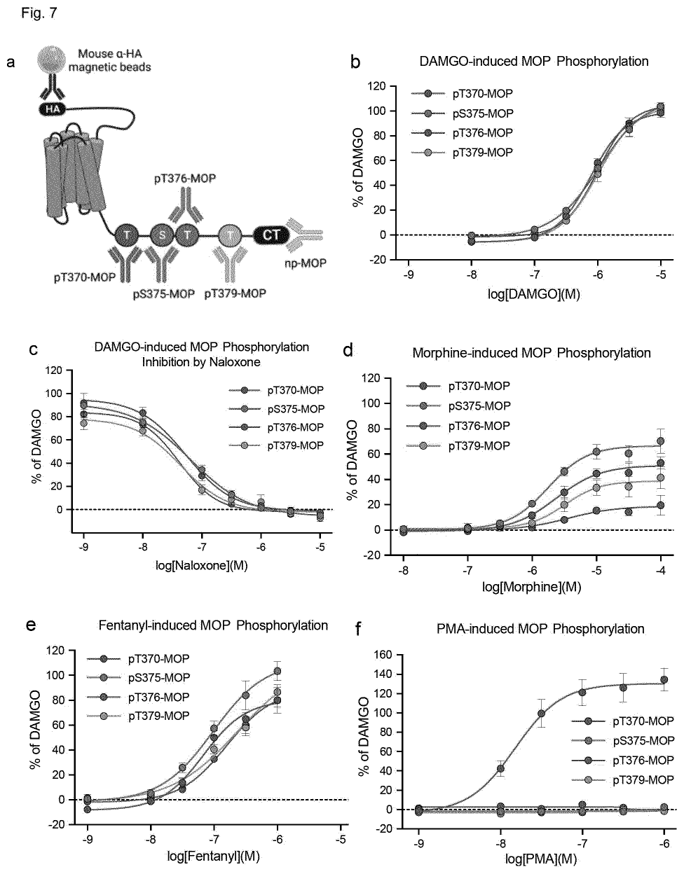

MOP activity is regulated by phosphorylation of four carboxyl-terminal serine and threonine residues, namely, T370, T376 and T379 in addition to S375. To facilitate a detailed assessment of agonist-selective phosphorylation signatures, the inventors generated phosphosite-specific antibodies and validated MOP phosphorylation immunoassay results for each of these sites. The inventors then evaluated a number of chemically diverse agonists using the enkephalin derivative DAMGO as the gold standard to calculate concentration-response curves. As depicted in FIGS. 7a and b, DAMGO stimulated phosphorylation with similar efficacy and potency at all four sites. DAMGO-induced MOP phosphorylation was inhibited by the antagonist naloxone in a concentration-dependent manner (FIG. 7c). In contrast to the effect of DAMGO, the partial agonist morphine induced S375 phosphorylation and, with much lower potency, phosphorylation at T370, T379 and T376 (FIG. 7d). The full agonist fentanyl promoted phosphorylation of all four sites with higher efficacy than DAMGO (FIG. 7e). In addition to homologous GRK-mediated phosphorylation, MOP also undergoes heterologous second messenger kinase-mediated phosphorylation, e.g. by protein kinase C (PKC) (Illing, S., Mann, A. & Schulz, S. Br. J. Pharmacol. 171, 1330-1340 (2014).). Notably, when PKC was activated by increasing concentrations of phorbol 12-myristate 13-acetate (PMA), the inventors found potent and selective phosphorylation of T370 (FIG. 7f). Thus, these findings recapitulated those from previous Western blot analyses, suggesting that the MOP phosphorylation immunoassay allows rapid and quantitative assessment of agonist-specific phosphorylation patterns. In addition, antagonist activity was evaluated, homologous and heterologous receptor phosphorylation was differentiated. From this set of measurements, the inventors calculated interassay and intra-assay coefficients of variability (CVs). For the pT370, pS375, pT376, and pT379 MOP phosphorylation assays, the interassay CV was generally at or less than 15%. The intra-assay CV was less than 10%, reflecting the high precision of these assays. In addition, the calculated Z′ factors for these assays ranged from 0.7 to 0.85, indicating their suitability for high-throughput applications. The inventors then performed β-galactosidase complementation assays to study the interrelation between GRK engagement, MOP phosphorylation and arrestin recruitment. In response to DAMGO, GRK2 and GRK3 were recruited with high efficiency, resulting in full phosphorylation of MOP. In contrast, the morphine-activated receptor recruited GRK2 and GRK3 with much lower potency, which led to only partial MOP phosphorylation. The concentration-response curves for β-arrestin1 and β-arrestin2 recruitment were virtually identical to those of DAMGO-induced MOP phosphorylation, whereas morphine-induced MOP phosphorylation was not sufficient to stimulate detectable mobilization of arrestins under these conditions. When activation of G protein-coupled inwardly rectifying potassium channels (GIRK) was measured using a membrane potential assay, the inventors observed a leftward shift in the concentration-response curves. This leftward shift reflects an amplification mechanism often detected with G protein assays, whereas complementation and phosphorylation assays are based on equimolar stoichiometric interactions of the protein partners.

### Example 8: Identification of GRKs in C5a1 Phosphorylation Assays

Next, the inventors tested whether phosphorylation assays can be used for assessing other GPCRs.

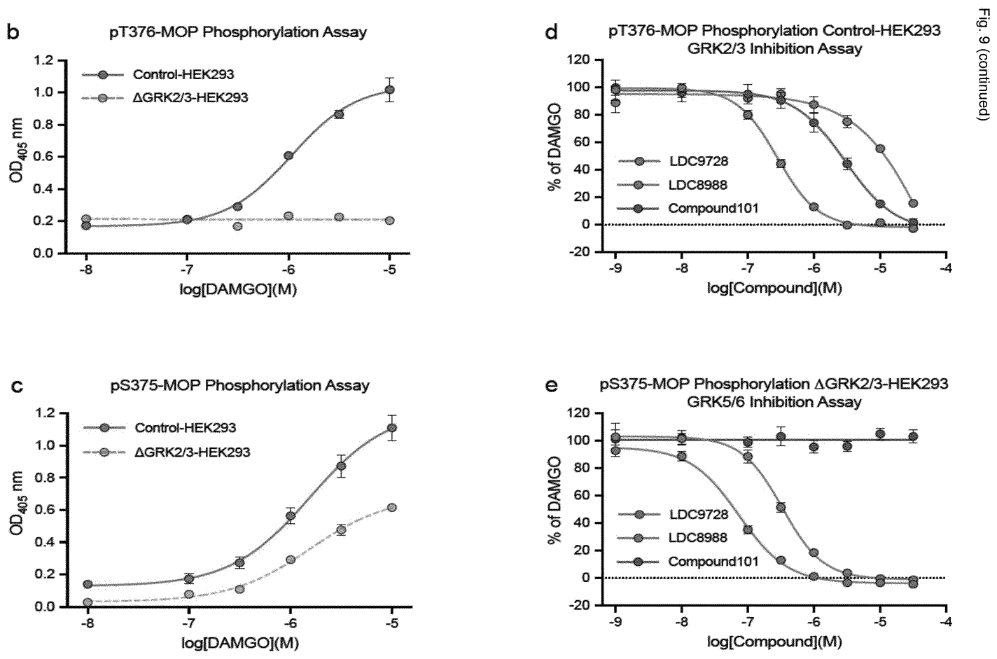

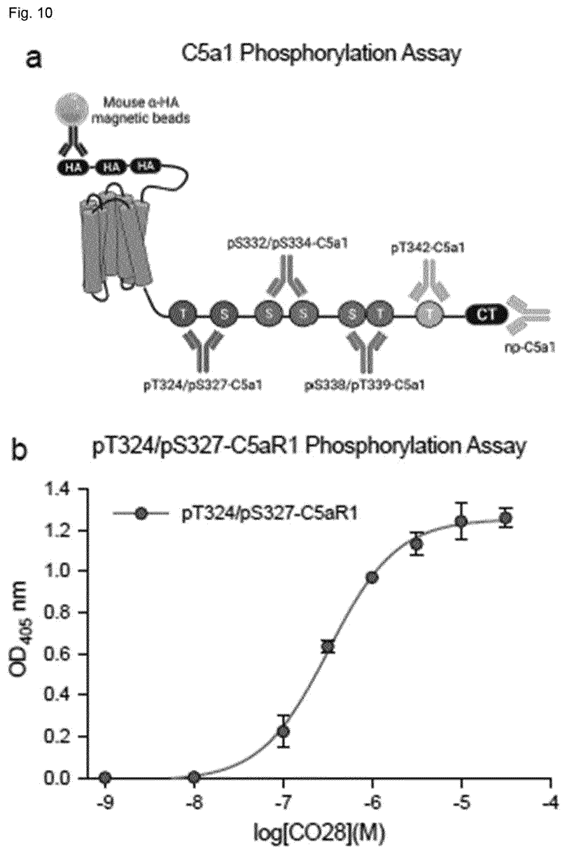

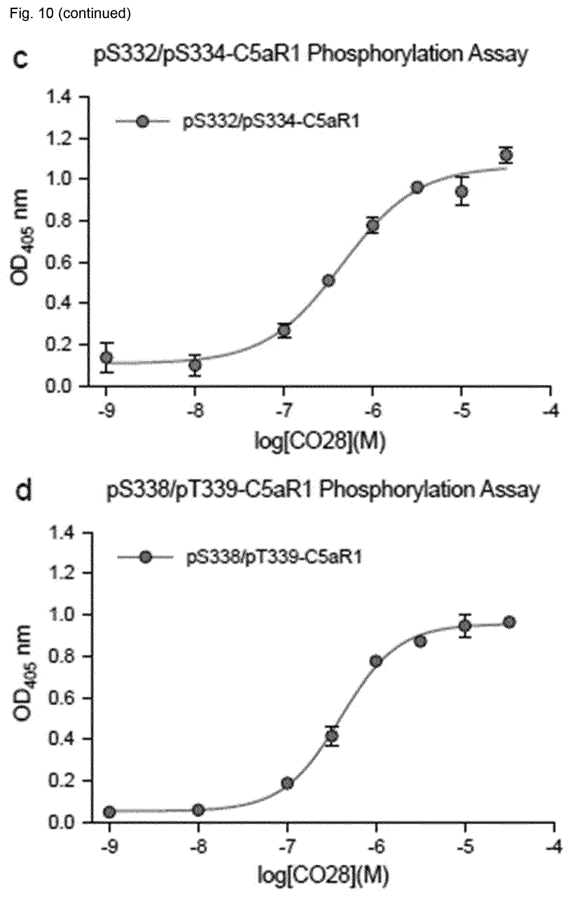

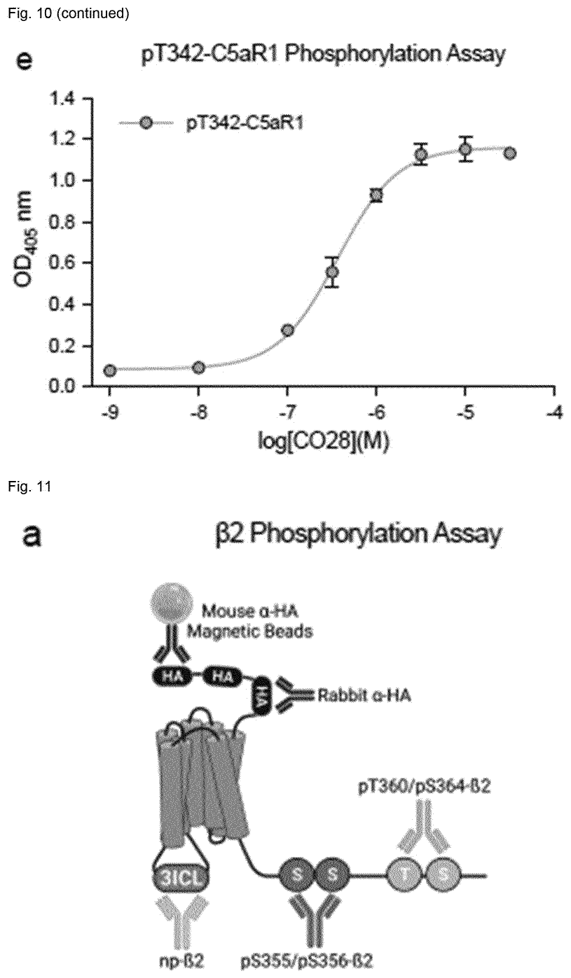

Complement component 5a receptor 1 (C5a1) is a prototypical receptor that is regulated by four distinct carboxyl-terminal phosphorylation sites or clusters, namely, T324/S327, S332/S334, S338/T339 and T342. Recent work with GRK-knockout cell clones showed that both GRK2/3 and GRK5/6 contributed equally to β-arrestin recruitment; however, the contribution of these GRKs to the phosphorylation of individual sites remained unclear. Therefore, the inventors generated and characterized phosphosite-specific antibodies for these sites and validated the corresponding pT324/pS327-, pS332/pS334-, pS338/pT339- and pT342-C5a1 immunoassays (FIG. 10). The inventors also established two alternative methods for detecting total receptor content to subsequently perform a quantitative phosphorylation analysis. First, the inventors generated an anti-non-phospho-(p)-C5a1 antibody (np-C5a1) that detects the receptor in the carboxyl-terminal region in a phosphorylation-independent manner. Second, the inventors expressed a receptor construct that contained three consecutive affinity tags in the N-terminus, which facilitated immunoprecipitation using mouse anti-HA antibody magnetic beads and simultaneous detection using rabbit anti-HA antibodies. The same receptor construct was then expressed in HEK293 cell clones in which GRK2/3 (ΔGRK2/3-HEK293) or GRK5/6 (ΔGRK5/6-HEK293) expression was knocked out, as well as in the parental HEK293 cell line (Control-HEK293). The cells were stimulated with the synthetic C5a1 agonist C028. The resulting concentration-response curves showed that for the phosphorylation of proximal sites T324/S327 and S332/S334, the GRK5/6 isoforms were predominantly required, whereas both the GRK2/3 and GRK5/6 isoforms contributed equally to phosphorylation of the distal sites S338/pT339 and T342 (FIG. 8a-d). These results suggest that 7TM phosphorylation assays can be performed to reveal the differential involvement of individual GRKs in generating agonist-selective phosphorylation barcodes and therefore can provide details in addition to those obtained with conventional β-arrestin recruitment assays.

### Example 9: GRK Inhibitor Screening Assays

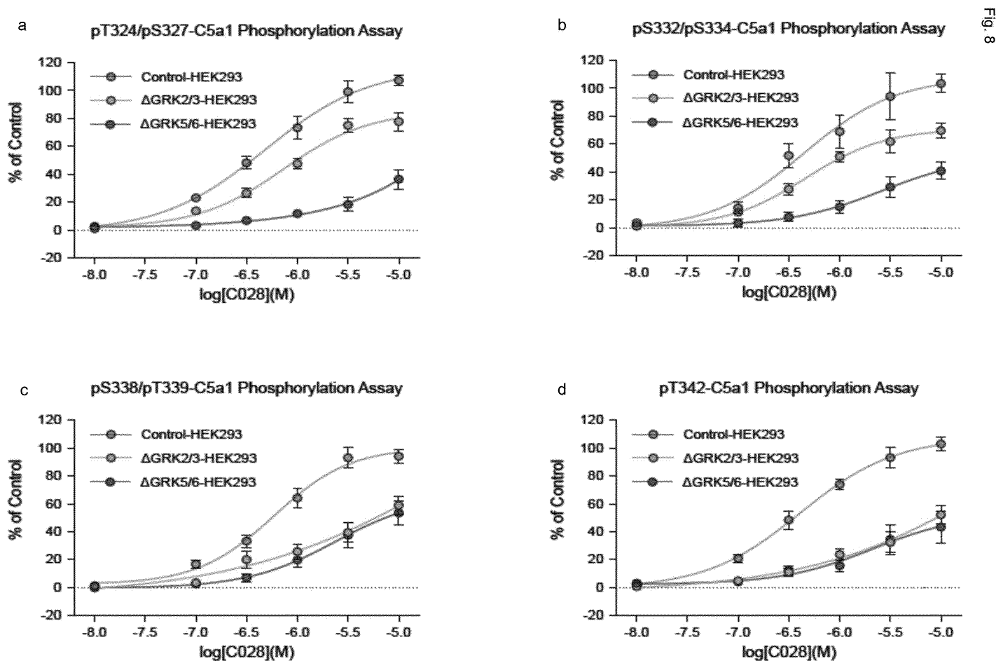

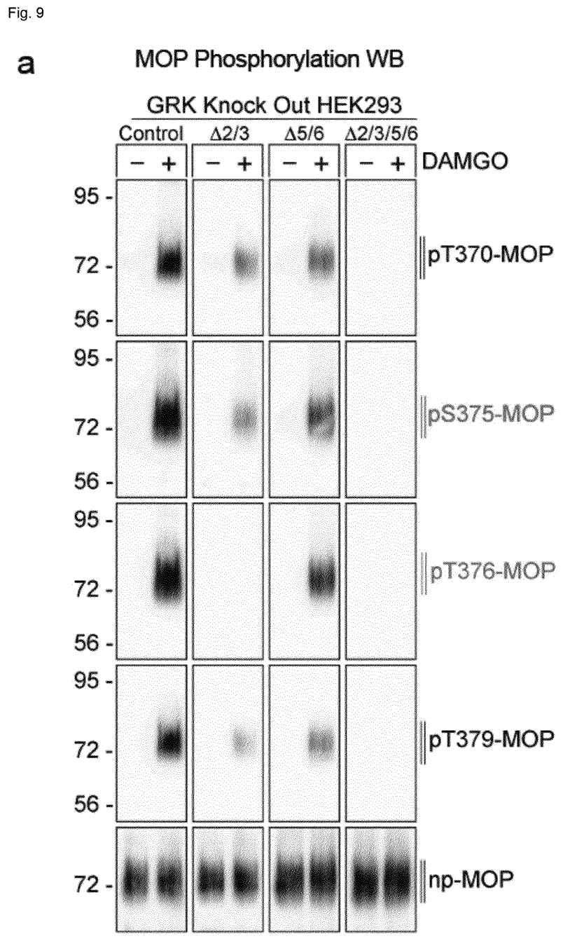

Screening for selective and membrane-permeable GRK inhibitors is still very challenging. Therefore, the inventors performed a set of experiments to establish cell screening assays for identifying novel GRK inhibitors. To this end, the inventors capitalized on a recently developed panel of combinatorial HEK293 cell clones in which GRK2/3/5/6 expression was knocked out in various combinations, with one, two, three or all genes knocked out (Drube, J. et al. Nat. Commun. 13, 540 (2022)). First, the inventors evaluated MOP phosphorylation through Western blot analysis. Phosphorylation was not detected in the quadruple ΔGRK2/3/5/6-HEK293 cells, confirming that DAMGO-induced MOP phosphorylation was entirely mediated by GRKs (FIG. 9a). The results depicted in FIG. 9 a reveal that T376-MOP phosphorylation was virtually absent in the ΔGRK2/3-HEK293 cells, suggesting that T376-MOP phosphorylation in parental Control-HEK293 cells was predominantly mediated by GRK2 and GRK3. In contrast, S375-MOP phosphorylation was clearly detectable in ΔGRK2/3-HEK293 cells, suggesting that residual S375-MOP phosphorylation was mediated by GRK5 and GRK6 (FIG. 9a). These findings were recapitulated with MOP phosphorylation immunoassays, indicating that pT376 MOP phosphorylation in Control-HEK293 cells can be used to screen for GRK2/3 inhibitors (FIGS. 9b and c). Conversely, pS375-MOP phosphorylation in ΔGRK2/3-HEK293 cells can be used to screen for GRK5/6 inhibitors (FIGS. 9b and c). Next, the inventors validated this hypothesis using compound 101, which is the most potent and selective GRK2/3 inhibitor available to date. In fact, compound 101 inhibited GRK2/3 activity with a half-maximal inhibitory concentration (IC₅₀) of 2.9 μM, but it had no effect on GRK5/6 (FIGS. 6d and e). The inventors then screened newly synthesized GRK inhibitors, which led to the discovery of LDC9728, which was 10-fold more effective than compound 101 in inhibiting GRK2/3 activity (FIG. 9d). LDC9728 was also highly effective in blocking GRK5/6 activity, suggesting that this compound is a highly potent pan-GRK inhibitor (FIG. 9e). In addition, the inventors discovered LDC8988 as the first potent and selective GRK5/6 inhibitor that has very little effect on GRK2/3 activity (FIGS. 9d and e). These results show that phosphorylation immunoassays can be used to identify relevant GRKs involved in receptor phosphorylation as well as to screen for novel GRK inhibitors.

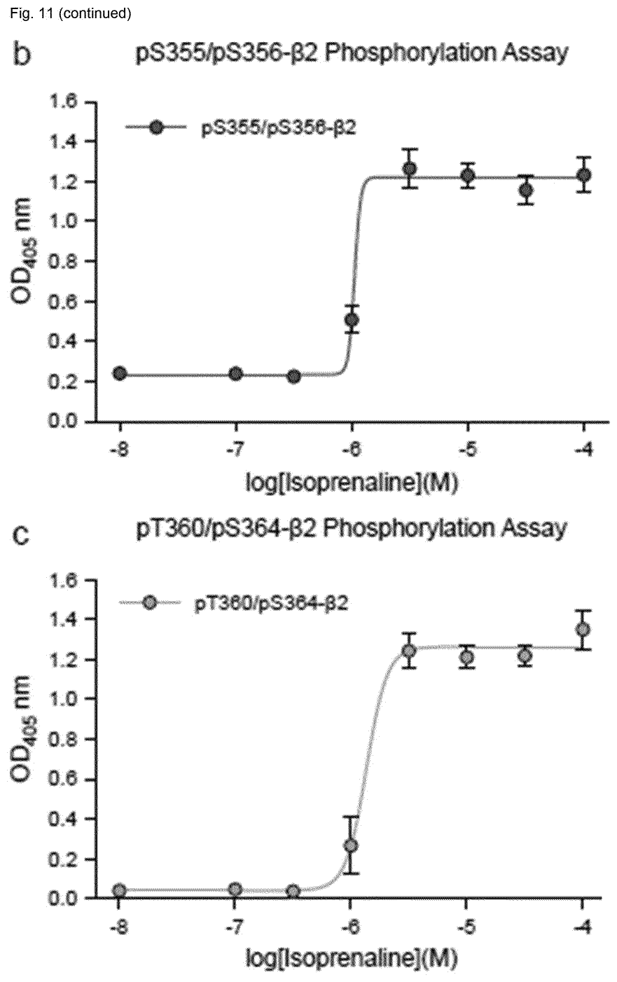

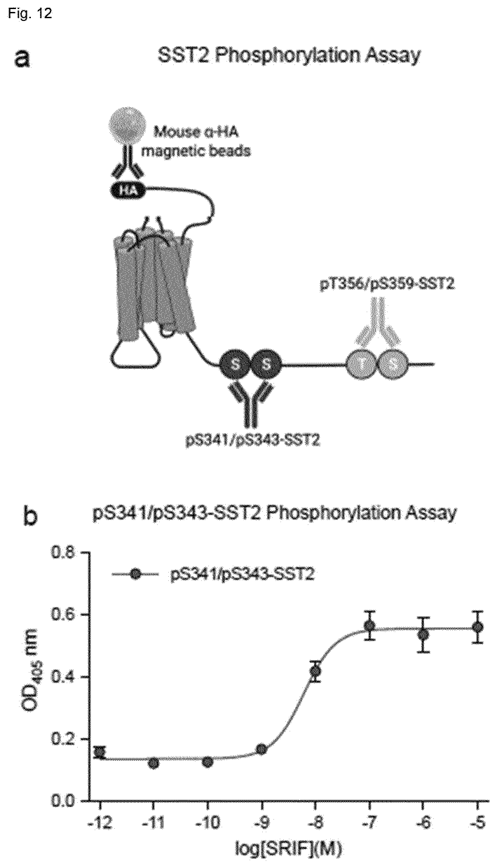

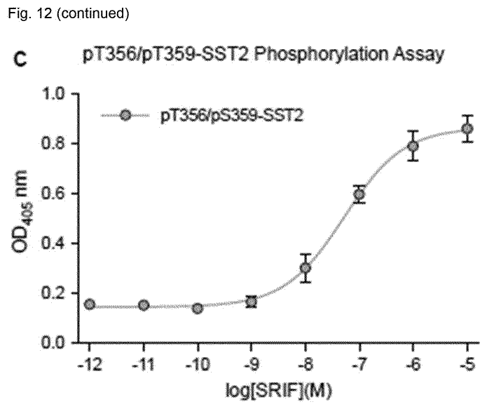

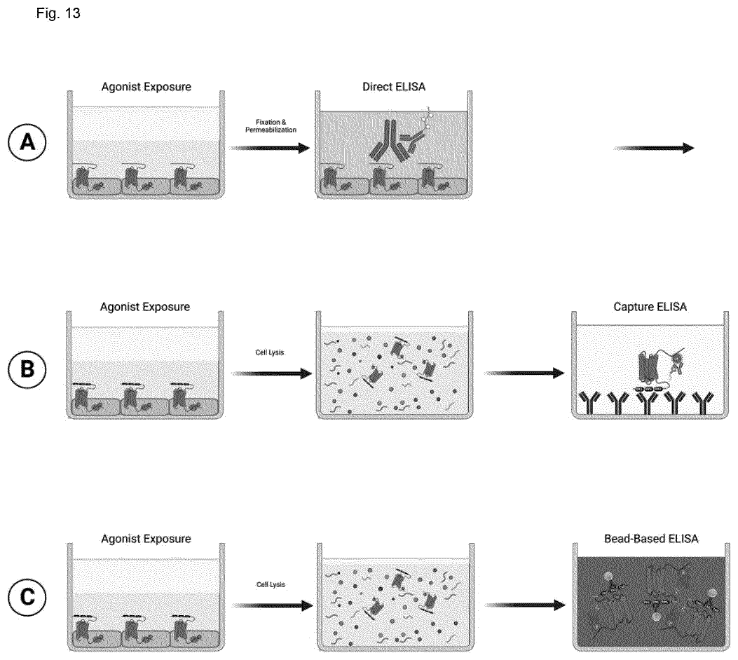

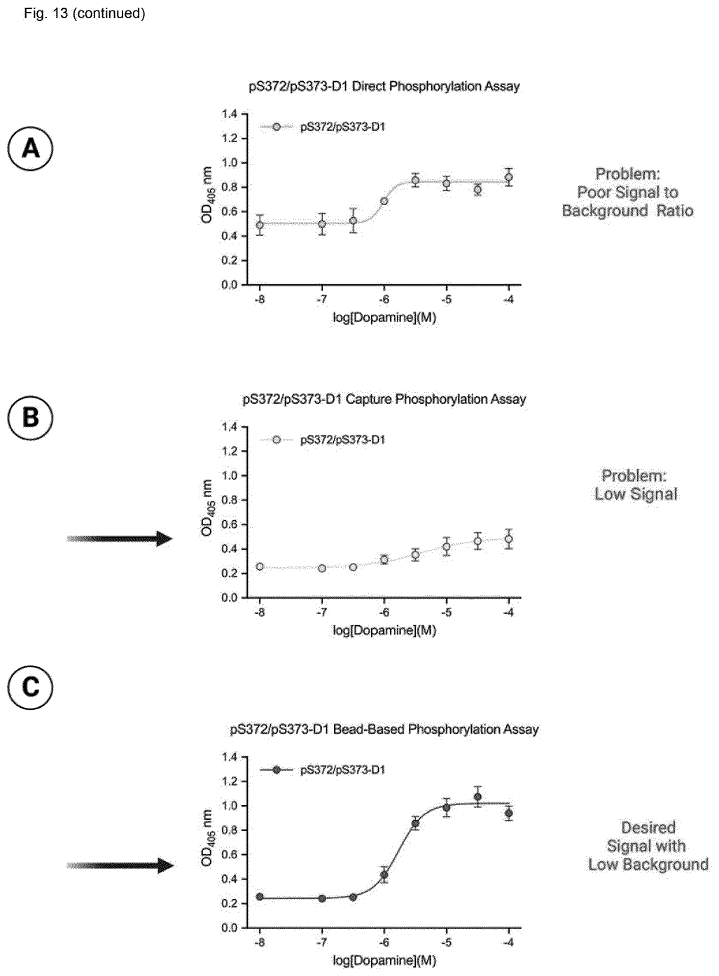

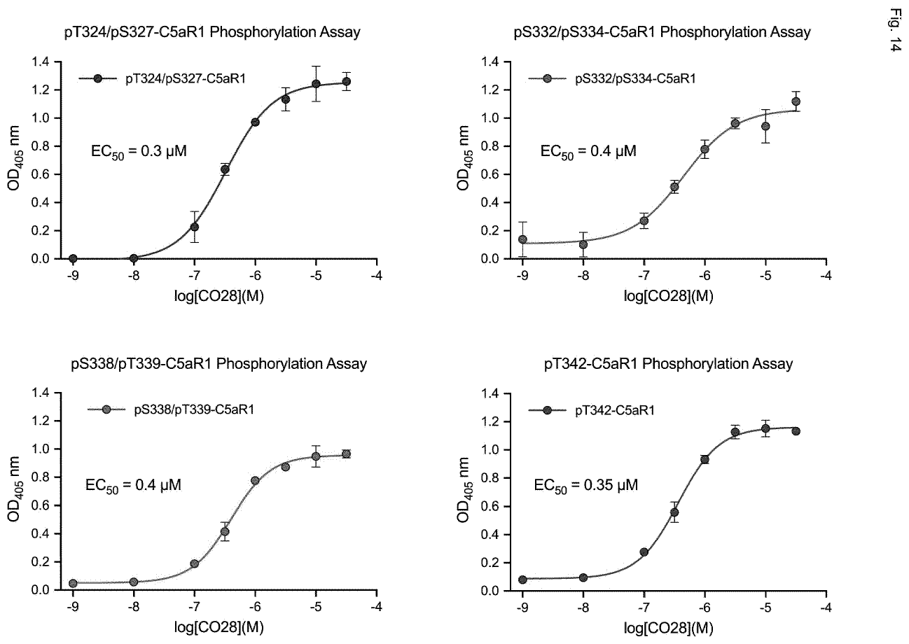

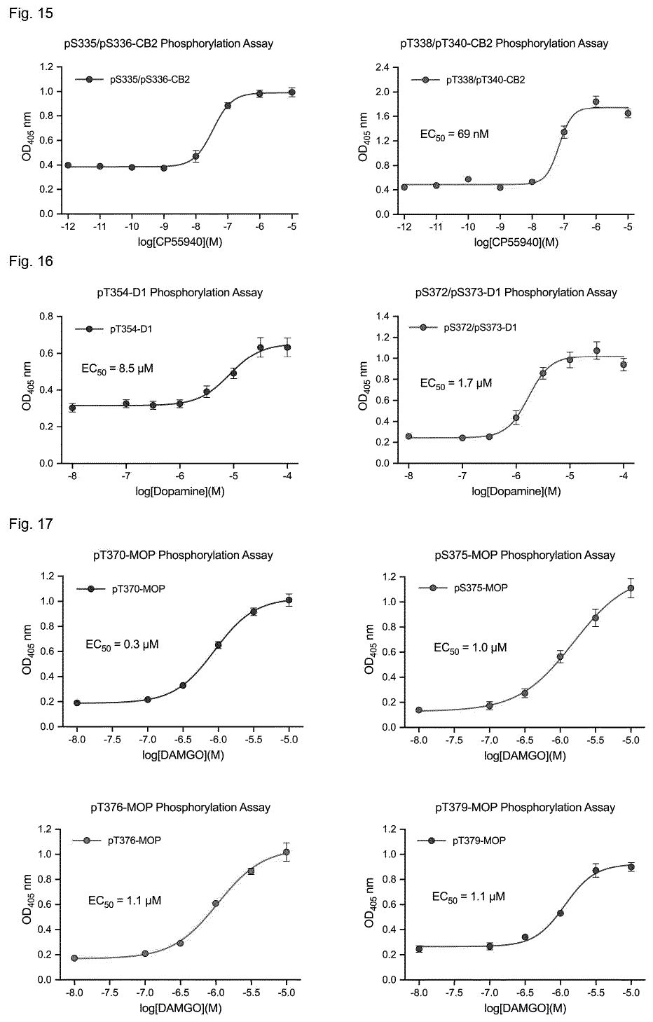

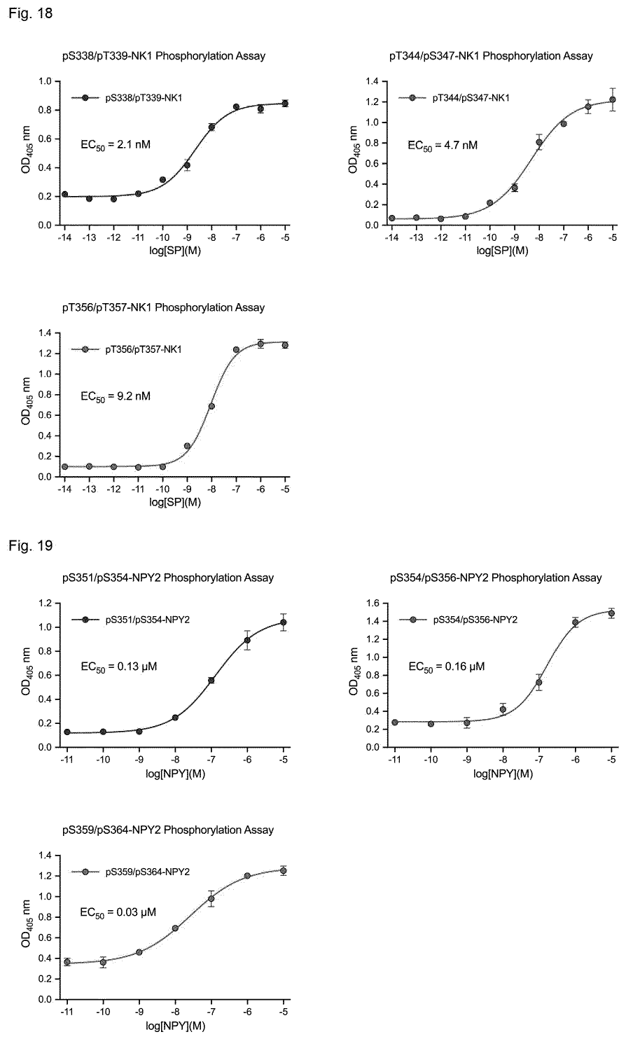

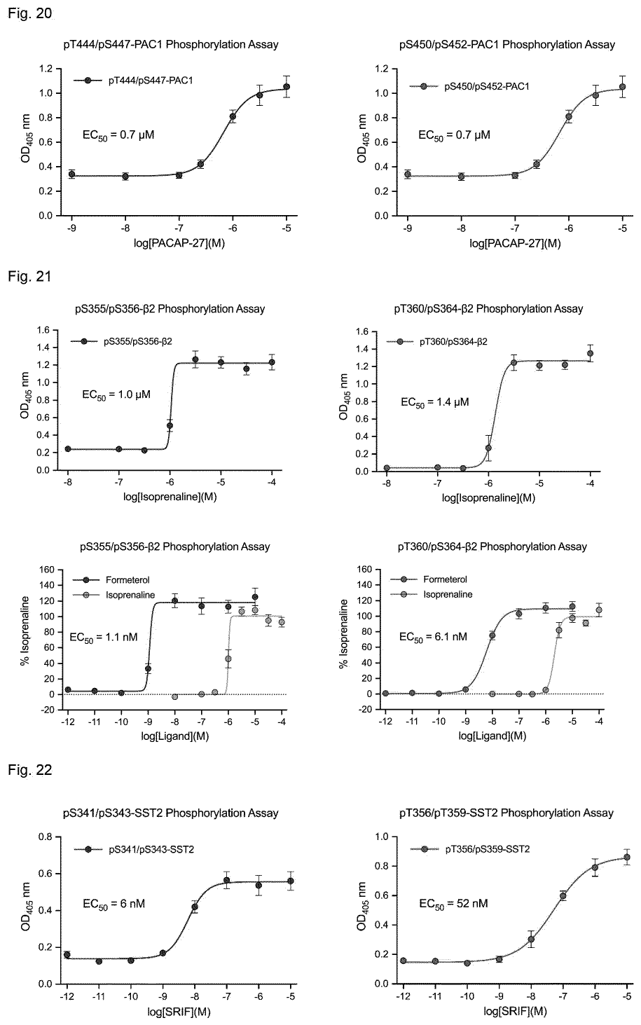

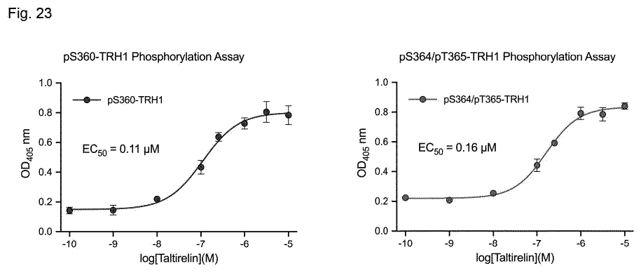

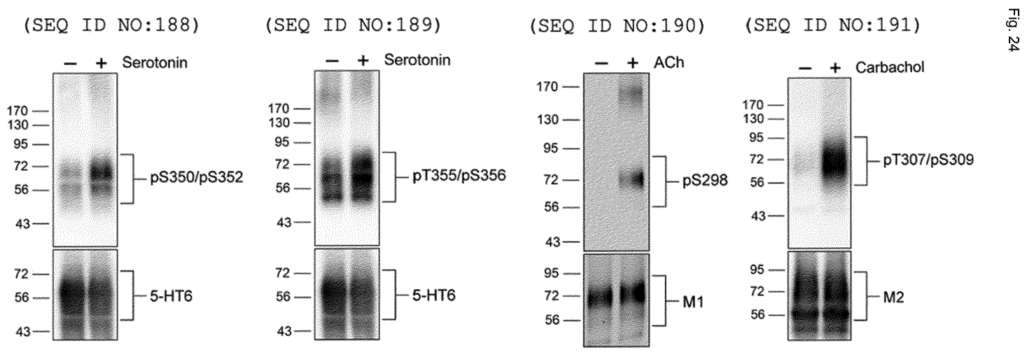

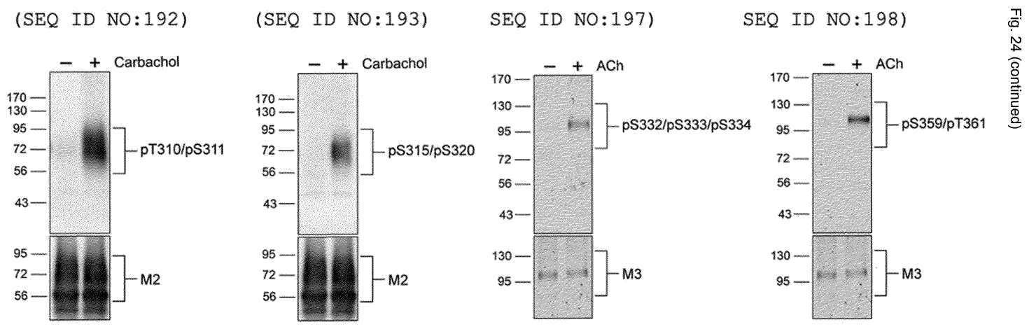

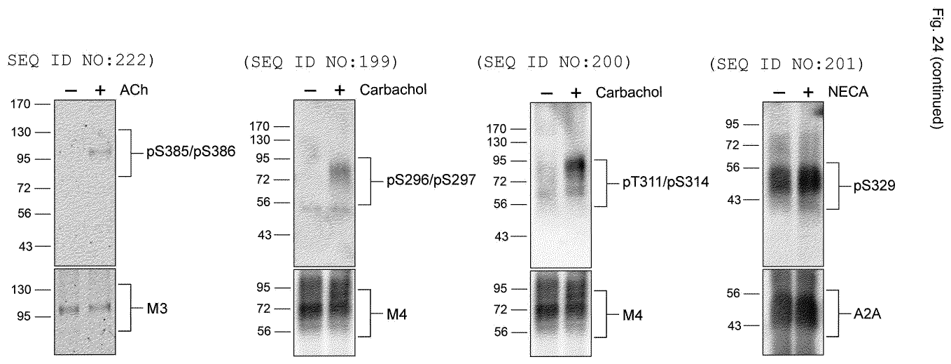

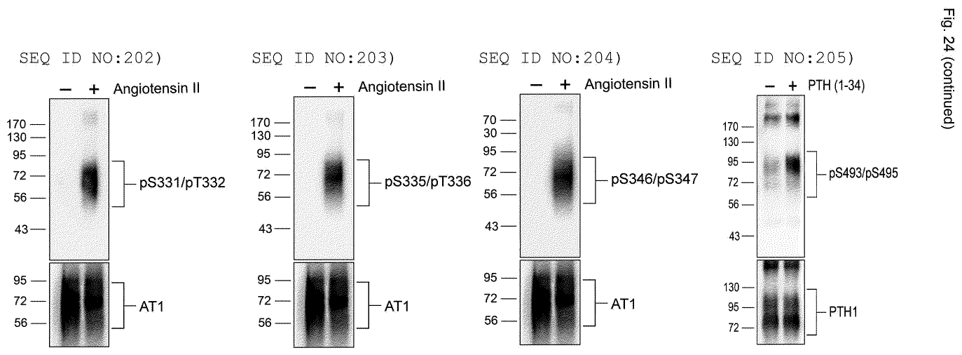

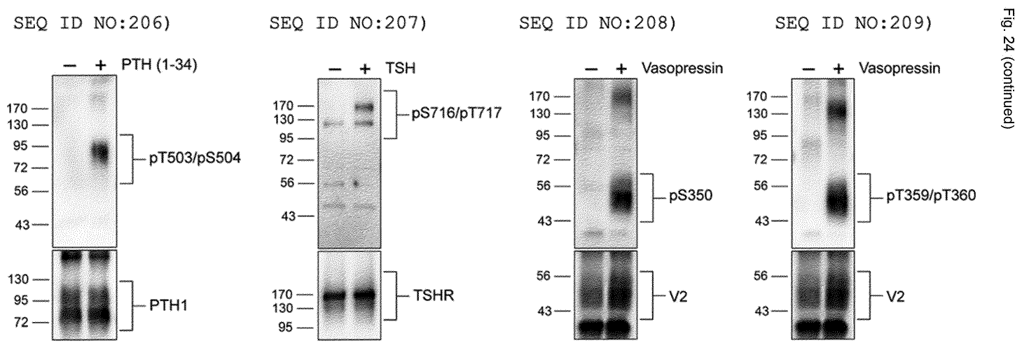

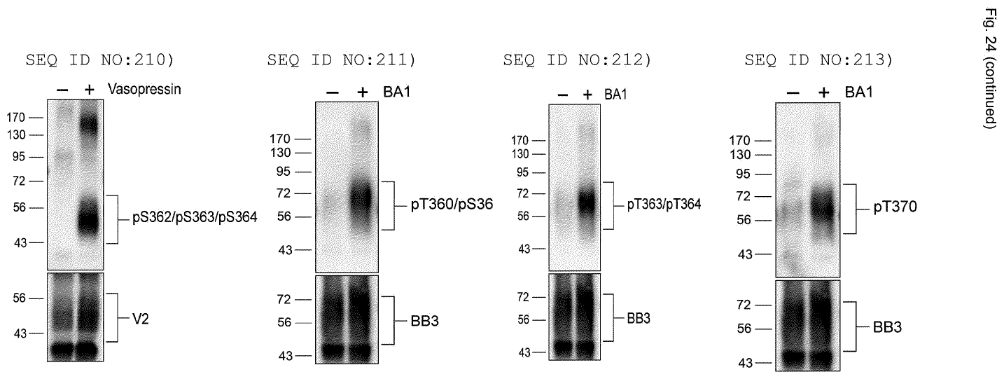

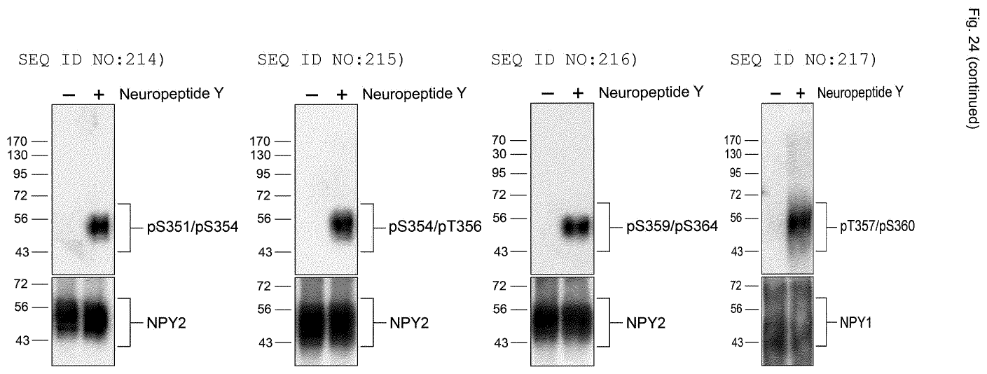

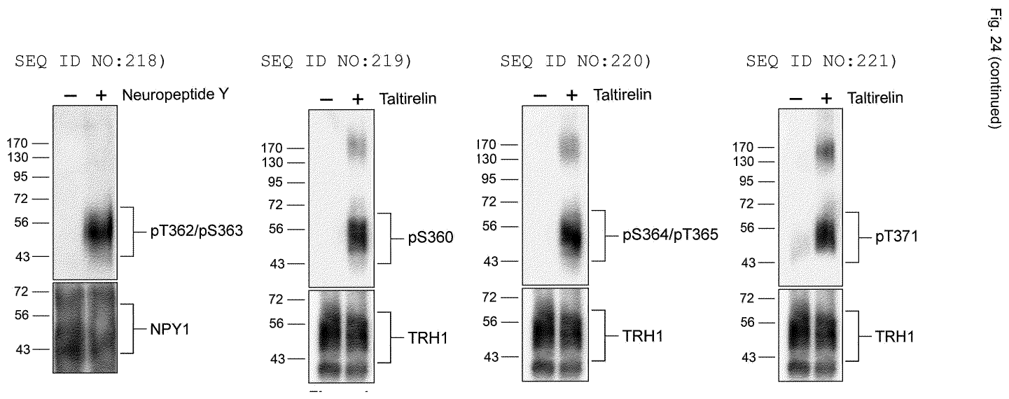

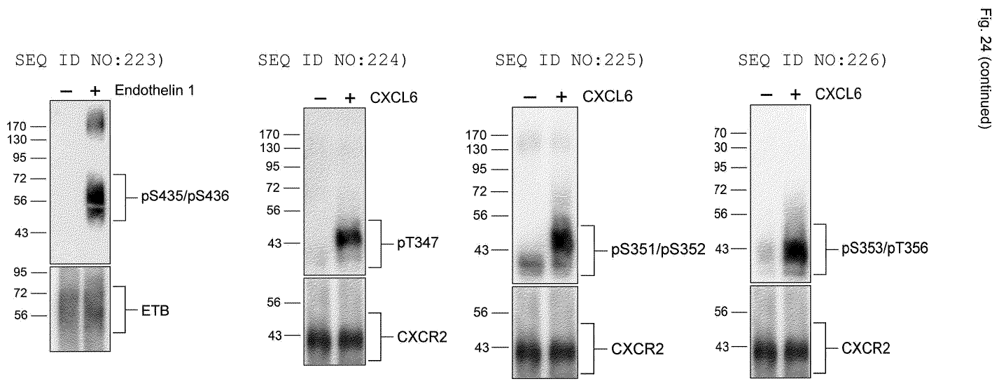

The final experiments were designed to assess the utility of available phosphosite-specific antibodies for the development of phosphorylation assays to identify other GPCR targets. In fact, the inventors rapidly established additional assays for B2-adrenoceptor (B2) (FIG. 11), demonstrating the versatility of the novel 7TM phosphorylation immunoassay.

### Example 10: Methods

### Antibodies, Cells and Reagents

The phosphosite-specific MOP antibodies against pT370-MOP (7TM0319B), PS375-MOP (7TM0319C), pT376-MOP (7TM0319D), and pT379-MOP (7TM0319E); the phosphosite-specific C5a1 antibodies against pT324/pS327-C5a1 (7TM0032A), pS332/pS334-C5a1 (7TM0032B), pS338/pT339-C5a1 (7TM0032D) and pT342-C5a1 (7TM0032E); and phosphosite-specific B2 antibodies against pS355/pS356-B2 (7TM0029A) and pT360/pS364-2 (7TM0029B), as well as the phosphorylation-independent antibodies against np-MOP (7TM0319N), np-C5a1 (7TM0032N), np-B2 (7TM0029N); and rabbit polyclonal anti-HA antibodies (7TM000HA) were provided by 7TM Antibodies (Jena, Germany) (www.7tmantibodies.com). All phosphosite-specific antibodies were affinity-purified against their immunizing phosphorylated peptides and subsequently cross-adsorbed against the corresponding unphosphorylated peptides. HEK293 cells were originally obtained from DSMZ Germany (ACC 305). Stable MOP-, C5a1- or ß2-expressing cells and GRK-knockout cells were generated as previously described (Drube, J. et al. Nat Commun 13, 540 (2022)). The plasmid encoding murine MOP with one amino-terminal HA-tag was custom-synthesized by imaGenes (Berlin, Germany). The plasmids encoding human C5a1 or B2 with three amino-terminal HA-tags were obtained from the cDNA resource center (C5R010TN00, AR0B20TN00) (www.cDNA.org). The GRK inhibitors LDC9728 and LDC8988 were provided by the Lead Discovery Center (Dortmund, Germany). The chemical structures for LDC9728 and LDC8988 will be reported in a separate publication.

### Cell Culture

HEK293 cells were cultured in Dulbecco's modified Eagle's medium (DMEM; Capricorn Scientific, DMEM-HXA) supplemented with 10% fetal calf serum (FCS, Capricorn Scientific FBS-11A) and a 1% penicillin and streptomycin mixture (Capricorn Scientific PS-B) at 37° C. with 5% CO2. Cells were passaged every 3-4 days and regularly checked for mycoplasma infections using a GoTaq G2 Hot Start Taq Polymerase kit from Promega.

### 7TM Phosphorylation Assay

The 7TM phosphorylation assay was performed according to the protocol outlined in FIG. 1 with either wild-type (Control-HEK293) or GRK-knockout HEK293 cells stably transfected with MOP, C5a1 or β2. The cells were seeded into 96-F-bottom-well cell culture microplates (Greiner Bio-One 655180) coated with 0.2 μg/ml poly-L-lysine (Sigma-Aldrich P1274) at a density of 80,000-100,000 cells per well and grown overnight to 95% confluency. The cells were then stimulated with the respective agonists for 30 min at 37° C. MOP was stimulated with DAMGO (Sigma-Aldrich E7384, in water), morphine (Hameln 03763738, in water), fentanyl (B. Braun 06900650, in water) or PMA (Tocris 1201, in dimethyl sulfoxide (DMSO)). C5a1 was stimulated with the FKP-(D-Cha)-Cha-r peptide agonist C028 (AnaSpec AS65121, in water). 32 was stimulated with isoprenaline (Sigma-Aldrich 15627, in water). In antagonist and GRK inhibitor experiments, naloxone (Ratiopharm 04788930, in water), compound 101 (Hello Bio HB2840, in DMSO), LDC9728 (Lead Discovery, in DMSO) or LDC8988 (Lead Discovery, in DMSO) was added 30 min before DAMGO stimulation. After washing with PBS, the cells were lysed in 150 μl/well detergent buffer (150 mM NaCl; 50 mM Tris-HCl, pH 7.4; 5 mM EDTA; 1% Igepal CA-360; 0.5% deoxycholic acid; and 0.1% SDS) supplemented with protease and phosphatase inhibitor cocktails (Roche #04693132001 and #04906845001, respectively) on an orbital shaker for 30 min at 4° C. In dephosphorylation experiments, phosphatase inhibitors were omitted from the indicated samples. Lysates were cleared by centrifugation at 3,700×g for 20 min at 4° C. using a microplate centrifuge (Thermo Fisher Multifuge X Pro). The supernatants were then transferred into 96-U-bottom-well assay plates (Greiner Bio-One 650101). Each sample was divided and placed into two corresponding wells of the assay plate for parallel detection of phosphorylated (80 μl of lysate per well) and total (40 μl of lysate per well) receptor levels. For in vivo phosphorylation experiments, knock-in mice expressing HA-MOP (Oprm1^{em1Shlz}, MGI: 6117675) were treated and brain lysates were prepared as described (Fritzwanker, S. et al. Commun Biol 4, 1070 (2021).). Mouse anti-HA magnetic beads (2 μl/well) (Thermo Fisher 88837) were washed in detergent buffer, diluted and vortexed for more accurate pipetting. A total volume of 40 μl of the bead slurry, which contained 20 μg magnetic beads with a diameter of 1 μm, was then added to each well. Assay plates were incubated on a microplate shaker (Corning LSE, digital) at 500-700 rpm for 2 h at 4° C. The beads were washed three times with 150 μl of PBS with 0.1% Tween 20 (PBS-T) under magnetic force. In the initial experiments, magnetic force was applied by placing the assay plates on a handheld magnetic separation block (V&P Scientific 771HH-H/Millipore 40-285). In all subsequent experiments, an automated microplate washer (BioTek 405TM TS Microplate Washer) was used. After the first washing cycle, primary rabbit phosphosite-specific and phosphorylation-independent antibodies were added at a final concentration of 3-5 μg/ml in 60 μl of PBS-T. The plates were then incubated either for 2 h at room temperature or overnight at 4° C. on a microplate shaker. In peptide neutralization experiments, phosphosite-specific antibodies were incubated with 1 μg/ml phosphorylated peptide used for immunizations or the corresponding unphosphorylated peptide for 1 h at room temperature on a turning wheel. After the second washing cycle, anti-rabbit horseradish peroxide (HRP)-linked secondary antibody (Cell Signaling Technology #7074) was added to PBS-T to a final dilution of 1:300 and incubated on a microplate shaker for 2 h at room temperature. After the third washing cycle, 100 μl of Super AquaBlue detection solution (Thermo Fisher 00-4203-58), an enhanced ABTS substrate solution, was added to each well, and the plates were incubated for 2-8 min until an OD at 405 nm (OD₄₀₅) between 1.0 and 1.4. The color reaction was stopped by the addition of 100 μl of 0.625 M oxalic acid. The assay plates were then placed on a magnetic separation block, and 150 μl of the supernatant was transferred to a transparent 96-well F-bottom detection plate (Greiner Bio-One 655182). The OD₄₀₅ was then determined using a FlexStation 3 microplate reader (Molecular Devices). Data were acquired with SoftMax Pro 5.4 software and analyzed with Excel 16.0 software. First, the mean of all background controls (without primary antibodies) was subtracted from all values. To normalize the signal intensity, the phosphorylation signal was multiplied by the quotient of the mean of all loading controls divided by the respective loading control. Therefore, the result was adjusted to the amount of receptor for each corresponding sample. This method provides the required information to create quantitative concentration-response curves based on raw OD₄₀₅ values. In each plate, samples of the control agonist were included to facilitate calculation of the results as a percent of agonist control. Values were displayed as concentration-response-curves of at least five independent experiments performed in duplicates and generated with GraphPad Prism 9.3.1 software.

### Western Blot Analysis

For dephosphorylation and detection limit experiments, HEK293 cells were grown, treated and processed as described for the 7TM phosphorylation assay until the first wash cycle of the magnetic beads was completed. Subsequently, 50 μl of 1×SDS sample buffer (100 mM DTT, 62.5 mM Tris-HCl, 20% glycerol, 2% SDS, and 0.005% bromophenol blue) was added to each well, and the plate was incubated for 25 min at 43° C. The assay plates were then placed on a magnetic separation block, and the supernatant was loaded onto an 8% polyacrylamide gel for immunoblot analysis (15 μl per lane). For all other experiments, cells were seeded in 60-mm dishes (Greiner Bio-One 628160) coated with 0.1 μg/ml poly-L-lysine (Sigma-Aldrich P1274) and grown to 90% confluency. The cells were then stimulated with an agonist for 30 min at 37° C. After washing with PBS, the cells were lysed in 800 μl of detergent buffer (150 mM NaCl; 50 mM Tris-HCl, pH 7.4; 5 mM EDTA; 1% Igepal CA-360; 0.5% deoxycholic acid; and 0.1% SDS) supplemented with protease and phosphatase inhibitor cocktails (Roche #04693132001 and #04906845001, respectively). After centrifugation at 14,000×g for 30 min at 4° C., lysates were incubated with 40 μl of mouse anti-HA agarose beads (Thermo Fisher 26182) at 4° C. on a turning wheel for 2 h. The beads were then washed three times with detergent buffer, 60 μl of 1×SDS sample buffer was added, and the samples were heated to 43° C. for 25 min to elute receptors from the beads. Supernatants were loaded onto 8% polyacrylamide gels. After gel electrophoresis, samples were blotted onto a PVDF membrane using a semidry electroblotting system. Phosphorylation was detected by incubating rabbit phosphosite-specific antibodies at concentrations of 1-2 μg/ml in 5% bovine serum albumin (BSA)/TBS overnight at 4° C. Signals were visualized with anti-rabbit HRP-linked secondary antibodies (Cell Signaling Technology #7074) and a chemiluminescence detection system (Thermo Fisher 34075). The blots were subsequently stripped and reprobed with a rabbit phosphorylation-independent antibody or rabbit anti-HA-tagged antibody to ensure equal loading of the gels. Western blot signals were imaged and quantified using a Fusion FX7 imaging system (Peqlab).

### Arrestin and GRK Binding Assays

Agonist-dependent binding of GRK2/3 and arrestin 1/2 to a MOP was determined using a β-galactosidase complementation assay as previously described (Miess, E. et al. Sci. Signal. 11, eaas9609 (2018).). HEK293 cells stably expressing MOP in which the C-terminal was fused with a β-Gal enzyme fragment (β-Gal1-44) and stably or transiently expressing β-arrestin1/2 or GRK2/3 fused to an N-terminal deletion mutant of β-Gal (β-Gal45-1043) were used. Receptor activation resulted in complementation of β-Gal fragments that generated an active enzyme. Thus, the enzyme activation levels are a direct result of MOP activation and are quantitated using a chemiluminescent β-Gal substrate (PJK Biotech #103312). Cells were plated in 48-well plates and grown for 48 h. After 60 min of agonist exposure, a cell lysis reagent was added, and luminescence was recorded with a FlexStation 3 microplate reader (Molecular Devices).

### G Protein-Gated Inwardly Rectifying Potassium Channel Assay

To analyze Gᵢ signaling, changes in membrane potential produced by activation of G protein-gated inwardly rectifying potassium channel (GIRK) were measured as previously described (Gunther, T., Culler, M. & Schulz, S. Mol. Endocrinol. 30, 479-490 (2016).; Dasgupta, P., Gunther, T., Reinscheid, R. K., Zaveri, N. T. & Schulz, S. Eur. J. Pharmacol. 890, 173640 (2021).). HEK293 cells were stably transfected with either HA-MOP or GFP-conjugated GIRK2 channel plasmids (OriGene). The cells were then seeded in 96-well plates and allowed to grow at 37° C. in 5% CO2 for 48 h. Hank's balanced salt solution (HBSS) with 20 mM HEPES solution (1.3 mM CaCl₂), 5.4 mM KCl, 0.4 mM K₂HPO₄, 0.5 mM MgCl₂, 0.4 mM MgSO₄, 136.9 mM NaCl, 0.3 mM Na₂HPO₄, 4.2 mM NaHCO₃ and 5.5 mM glucose; pH 7.4) was used to wash the cells. A membrane potential dye (FLIPR Membrane Potential kit BLUE, Molecular Devices R8034) was reconstituted according to the manufacturer's instructions. To each well, 90 μl of the HBSS/HEPES buffer solution and an equal volume of the membrane potential dye were added to a final volume of 180 μl per well. The cells were then incubated at 37° C. for 45 min. Test compounds were prepared in buffer solution containing HBSS and 20 mM HEPES solution (pH 7.4) at 10-fold the final concentration to be measured. Fluorescence measurements were performed with a FlexStation 3 microplate reader (Molecular Devices) at 37° C. with excitation at 530 nm and emission at 565 nm. Baseline readings were taken every 1.8 sec for 1 min. After 60 sec, 20 μl of the test or vehicle control was injected into each well containing cells incubated with the dye to a final in-well volume of 200 μl, which resulted in a 1:10 dilution of the test compound. The change in dye fluorescence was recorded for 240 sec with SoftMax Pro 5.4 software. Peak fluorescence values were obtained after subtraction of baseline readings for each sample and then used to calculate concentration-response curves using Origin.

### Saturation Binding Assay

Binding experiments were performed on membranes prepared from wild-type and HA-MOP-transfected HEK293 cells using [³H]DAMGO (51.7 Ci/mmol) (PerkinElmer NET902250UC) as described previously (Kaserer, T., Lantero, A., Schmidhammer, H., Spetea, M. & Schuster, D. Sci. Rep. 6, 21548 (2016).). Briefly, cells grown to confluency were harvested in PBS and stored at −80° C. Saturation binding experiments were performed with 50 mM Tris-HCl buffer (pH 7.4) in a final volume of 1 ml containing 30-40 μg of membrane protein. Membranes were incubated with different concentrations of [³H]DAMGO (0.05-9 nM) at 25° C. for 60 min. Nonspecific binding was determined in the presence of 10 μM unlabeled DAMGO. Reactions were terminated by rapid filtration through Whatman glass GF/C fiber filters. The filters were washed three times with 5 ml of ice-cold 50 mM Tris-HCl buffer (pH 7.4) with a Brandel M24R cell harvester. Bound radioactivity retained on the filters was measured by liquid scintillation counting using a Beckman Coulter LS6500. All experiments were repeated three times with independently prepared samples. Nonlinear regression analysis of the saturation binding curves was performed with GraphPad Prism.

### In Vitro GRK2 and GRK5 Kinase Assays

The kinase inhibitory activity of LDC8988 and LDC9728 against GRK2 and GRK5 was assessed using the Lance kinase activity assay, which is based on the detection of a phosphorylated Ulight-peptide substrate by a specific europium-labeled anti-phospho peptide antibody. Binding of a kinase inhibitor prevents phosphorylation of the Ulight-substrate resulting in loss of the FRET signal. The assay was performed at two different ATP concentrations, either representing the Kₘ of ATP for GRK2 and GRK5 or mimicking intracellular ATP levels. For IC₅₀ determination, 4 μl kinase working solution in assay buffer (50 mM HEPES pH 7.5, 10 mM MgCL₂, 1 mM EGTA, 0.01% Tween20, 1% DMSO, 2 mM DTT) and 4 μl of substrate working solution in assay buffer were transferred into assay plates (Corning #4513). Compound was added via an Echo acoustic dispenser (BeckmanCoulter) in a concentration range from 10 to 0.0025 μM. Reaction was started by addition of 2 μl ATP. After 1 h incubation at room temperature, the reaction was stopped with 10 μl detection mix containing the anti-phospho peptide antibody and 10 mM EDTA. After a second incubation period of 1 h at room temperature the FRET signal was measured at 340 nm excitation, 665 nm and 615 nm emission with an Envision spectrophotometer (Perkin Elmer) with 50 μs delay and 300 μs integration time. IC₅₀ values were calculated with concentration-response-curves with Quattro Workflow.

1. A method for determining a phosphorylation status of a 7TMR polypeptide, said method comprising the steps:
providing a cell comprising a 7TMR construct, wherein the 7TMR construct comprises a 7TMR polypeptide, and an affinity tag;
in a lysis step, lysing the cell with a lysis buffer yielding a cell lysate;
in a first contacting step, contacting the cell lysate with magnetic beads coated with a first binder;
in a second contacting step, contacting the magnetic beads with a second binder;
wherein one of the first and the second binder is capable of binding to the affinity tag, and the other one of the first and the second binder is a first antibody capable of specifically binding to said 7TMR polypeptide when said 7TMR polypeptide is in a phosphorylated status, but not in unphosphorylated status; and
wherein the second binder is coupled directly or indirectly to a detection moiety;
in a detection step, detecting the phosphorylation status of the 7TMR polypeptide by measuring a signal derived from the detection moiety.

2. The method according to claim 1, an inhibitor of phosphatases and/or an inhibitor of proteases.

3. The method according to claim 1, wherein after the lysis step, the cell lysate is centrifuged, and the supernatant is used in the contact step.

4. The method according to claim 1, wherein the detection moiety is selected from a fluorophore, and an enzyme catalyzing a reaction producing a dye, particularly an enzyme selected from horse-radish peroxidase (HRP) and alkaline phosphatase (AP).

5. The method according to claim 1, wherein the magnetic beads carry an isotope or a fluorescent label, more particularly a fluorescent label.

6. The method according to claim 5, wherein the magnetic beads are coupled to a plurality of first antibodies, and each first antibody of said plurality specifically binds to a different phosphorylated sequence and is bound to magnetic beads carrying a label characterized by a different isotope or a different fluorescent color.

7. The method according to claim 1, wherein the cell lysate is split into a first half and a second half after the lysis step, and wherein
the first half is contacted with said first antibody, and
the second half is contacted with a binder for the 7TMR, wherein the binder for the 7TMR does not depend on a phosphorylation status of the 7TMR,
and wherein a ratio between a signal of the first antibody and a signal of the binder for the 7TMR is indicative of an amount of phosphorylation of the 7TMR.

8. The method according to claim 1, wherein the 7TMR construct comprises a first affinity tag and a second affinity tag, and wherein
a binder of the first affinity tag is coupled to the magnetic beads, and
a binder of the second affinity tag is coupled directly or indirectly to a detection moiety,
and wherein a ratio between a signal of the first antibody and a signal of the binder of the second affinity tag is indicative of an amount of phosphorylation of the 7TMR.

9. A method for determining a phosphorylation status of a 7TMR polypeptide, said method comprising the steps:
providing a cell comprising a 7TMR construct, wherein the 7TMR construct comprises a 7TMR polypeptide, and an affinity tag;
in a lysis step, lysing the cell with a lysis buffer yielding a cell lysate;
in a binding step, contacting the sample with
i. an affinity binder; and
ii. a first antibody specifically binding to said 7TMR polypeptide in phosphorylated status, but not in unphosphorylated status;
wherein one of the affinity binder and the first antibody is coupled to an emission moiety, and the other one of the binder and the first antibody is coupled to an excitation moiety, wherein the emission moiety is capable of emitting light at a wavelength which corresponds to the excitation wavelength of the excitation moiety;
in a detection step, detecting the phosphorylation status of the 7TMR polypeptide via measuring a signal derived from the excitation moiety, optionally exciting the emission moiety with light of an appropriate wavelength.

10. The method according to claim 9, wherein the emission moiety and the excitation moiety are a FRET (Förster resonance energy transfer) pair or a BRET (bioluminescence resonance energy transfer) pair.

11. A method for determining an activity of a 7TMR ligand of interest, said method comprising the steps:
in a ligand step, contacting a cell comprising a 7TMR polypeptide with a 7TMR ligand of interest;
determining the phosphorylation status of said 7TMR polypeptide via the method of claim 1.

12. A method for identifying a kinase specific for a distinct 7TMR phosphorylation pattern, wherein a cell comprises a plurality of kinases specific for a 7TMR polypeptide, the method comprising the steps:
providing a plurality of cells, wherein each cell of said plurality comprises only one kinase of said plurality of kinases;
performing the method of claim 1 on all cells of said plurality;
determining which kinase of said plurality is specific for said distinct 7TMR phosphorylation pattern.

13. A method for identifying a 7TMR polypeptide activated by a ligand of interest, said method comprising the steps of the method of claim 11 being repetitively executed for a plurality of 7TMR polypeptides.

14. A method for identifying a modulator, particularly an inhibitor, of a kinase specific for a distinct 7TMR phosphorylation pattern, said method comprising the steps
providing a cell comprising a 7TMR construct, wherein the 7TMR construct comprises a 7TMR polypeptide, and an affinity tag;
contacting said cell with a compound of interest;
performing the method of claim 1 on said cell;
determining whether said compound of interest has an effect on a phosphorylation pattern of said 7TMR polypeptide, and thereby identifying a modulator of a kinase specific for a distinct 7TMR phosphorylation pattern.

15. The method according to claim 1, wherein the first antibody
i. is capable of specifically binding to
a. a target sequence if the target sequence is phosphorylated and the target sequence is selected from a group of sequences comprising SEQ ID NO 1-51 and SEQ ID NO 139-141 and SEQ ID NO 145-178,
particularly wherein the target sequence is phosphorylated in all phosphorylation sites,
or
b. a target sequence if the target sequence is phosphorylated in all indicated phosphorylation sites and the target sequence is selected from a group of sequences comprising SEQ ID NO 52-54 and SEQ ID NO 179-182,
ii. and is not capable of binding to the corresponding unphosphorylated sequence.
